SEQUENCE LISTING (47 sequences)

SEQ ID NO: 1 - DNA, 1422 bp, Mus musculus
  source 1..1422: mol_type = other DNA
atgcgactctctaaaacccttgccggcatggatatggccgactacagcgctgccctggacccagcctacaccaccctggagtttgaaaatgtgcaggtgttgaccatgggcaatgacacgtccccatctgaaggtgccaacctcaattcatccaacagcctgggcgtcagtgccctgtgcgccatctgtggcgaccgggccaccggcaaacactacggagcctcgagctgtgacggctgcaaggggttcttcaggaggagcgtgaggaagaaccacatgtactcctgcaggtttagccgacaatgtgtggtagacaaagataagaggaaccagtgtcgttactgcaggcttaagaagtgcttccgggctggcatgaagaaggaagctgtccaaaatgagcgggaccggatcagcacgcggaggtcaagctacgaggacagcagcctgccctccatcaacgcgctcctgcaggcagaggttctgtcccagcagatcacctctcccatctctgggatcaatggcgacattcgggcaaagaagattgccaacatcacagacgtgtgtgagtctatgaaggagcagctgctggtcctggtcgagtgggccaagtacatcccggccttctgcgaactccttctggatgaccaggtggcgctgctcagggcccacgccggtgagcatctgctgcttggagccaccaagaggtccatggtgtttaaggacgtgctgctcctaggcaatgactacatcgtccctcggcactgtccagagctagcggagatgagccgtgtgtccatccgcatcctcgatgagctggtcctgcccttccaagagctgcagattgatgacaatgaatatgcctgcctcaaagccatcatcttctttgatccagatgccaaggggctgagtgacccgggcaagatcaagcggctgcggtcacaggtgcaagtgagcctggaggattacatcaacgaccggcagtacgactctcggggccgctttggagagctgctgctgctgttgcccacgctgcagagcatcacctggcagatgatcgaacagatccagttcatcaagctcttcggcatggccaagattgacaacctgctgcaggagatgcttctcggagggtctgccagtgatgcaccccacacccaccaccccctgcaccctcacctgatgcaagaacacatgggcaccaatgtcattgttgctaacacgatgccctctcacctcagcaatggacagatgtgtgagtggccccgacccagggggcaggcagccactcccgagactccacagccatcaccaccaagtggctcgggatctgaatcctacaagctcctgccaggagccatcaccaccatcgtcaagcctccctctgccattccccagccaacgatcaccaagcaagaagccatc

SEQ ID NO: 2 - RNA, 1422 nt, Mus musculus
  source 1..1422: mol_type = mRNA
atgcgactctctaaaacccttgccggcatggatatggccgactacagcgctgccctggacccagcctacaccaccctggagtttgaaaatgtgcaggtgttgaccatgggcaatgacacgtccccatctgaaggtgccaacctcaattcatccaacagcctgggcgtcagtgccctgtgcgccatctgtggcgaccgggccaccggcaaacactacggagcctcgagctgtgacggctgcaaggggttcttcaggaggagcgtgaggaagaaccacatgtactcctgcaggtttagccgacaatgtgtggtagacaaagataagaggaaccagtgtcgttactgcaggcttaagaagtgcttccgggctggcatgaagaaggaagctgtccaaaatgagcgggaccggatcagcacgcggaggtcaagctacgaggacagcagcctgccctccatcaacgcgctcctgcaggcagaggttctgtcccagcagatcacctctcccatctctgggatcaatggcgacattcgggcaaagaagattgccaacatcacagacgtgtgtgagtctatgaaggagcagctgctggtcctggtcgagtgggccaagtacatcccggccttctgcgaactccttctggatgaccaggtggcgctgctcagggcccacgccggtgagcatctgctgcttggagccaccaagaggtccatggtgtttaaggacgtgctgctcctaggcaatgactacatcgtccctcggcactgtccagagctagcggagatgagccgtgtgtccatccgcatcctcgatgagctggtcctgcccttccaagagctgcagattgatgacaatgaatatgcctgcctcaaagccatcatcttctttgatccagatgccaaggggctgagtgacccgggcaagatcaagcggctgcggtcacaggtgcaagtgagcctggaggattacatcaacgaccggcagtacgactctcggggccgctttggagagctgctgctgctgttgcccacgctgcagagcatcacctggcagatgatcgaacagatccagttcatcaagctcttcggcatggccaagattgacaacctgctgcaggagatgcttctcggagggtctgccagtgatgcaccccacacccaccaccccctgcaccctcacctgatgcaagaacacatgggcaccaatgtcattgttgctaacacgatgccctctcacctcagcaatggacagatgtgtgagtggccccgacccagggggcaggcagccactcccgagactccacagccatcaccaccaagtggctcgggatctgaatcctacaagctcctgccaggagccatcaccaccatcgtcaagcctccctctgccattccccagccaacgatcaccaagcaagaagccatc

SEQ ID NO: 3 - protein, 474 aa, Mus musculus
MRLSKTLAGMDMADYSAALDPAYTTLEFENVQVLTMGNDTSPSEGANLNSSNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSQQITSPISGINGDIRAKKIANITDVCESMKEQLLVLVEWAKYIPAFCELLLDDQVALLRAHAGEHLLLGATKRSMVFKDVLLLGNDYIVPRHCPELAEMSRVSIRILDELVLPFQELQIDDNEYACLKAIIFFDPDAKGLSDPGKIKRLRSQVQVSLEDYINDRQYDSRGRFGELLLLLPTLQSITWQMIEQIQFIKLFGMAKIDNLLQEMLLGGSASDAPHTHHPLHPHLMQEHMGTNVIVANTMPSHLSNGQMCEWPRPRGQAATPETPQPSPPSGSGSESYKLLPGAITTIVKPPSAIPQPTITKQEAI

SEQ ID NO: 4 - DNA, 1392 bp, Homo sapiens
  source 1..1392: mol_type = other DNA
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatc

SEQ ID NO: 5 - RNA, 1392 nt, Homo sapiens
  source 1..1392: mol_type = mRNA
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatc

SEQ ID NO: 6 - protein, 464 aa, Homo sapiens
MRLSKTLVDMDMADYSAALDPAYTTLEFENVQVLTMGNDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMSTPETPQPSPPGGSGSEPYKLLPGAVATIVKPLSAIPQPTITKQEVI

SEQ ID NO: 7 - RNA, 151 nt, synthetic construct
  source 1..151: mol_type = other RNA
taatacgactcactataagggagacccaagctggctagcgtttaaacttaagcttgaataaactagtattcttctggtccccacagactcagagagaacccgccaccaagggagactcttctggtccccacagactcagagagaacccacc

SEQ ID NO: 8 - RNA, 110 nt, synthetic construct
  source 1..110: mol_type = other RNA
gctggagcctcggtggccatgcttcttgccccttgggcctccccccagcccctcctccccttcctgcacccgtacccccgtggtctttgaataaagtctgagtgggcggc

SEQ ID NO: 9 - DNA, 1686 bp, synthetic construct
  source 1..1686: mol_type = other DNA
taatacgactcactataagggagacccaagctggctagcgtttaaacttaagcttgaataaactagtattcttctggtccccacagactcagagagaacccgccaccaagggagactcttctggtccccacagactcagagagaacccaccatgcgactctctaaaacccttgccggcatggatatggccgactacagcgctgccctggacccagcctacaccaccctggagtttgaaaatgtgcaggtgttgaccatgggcaatgacacgtccccatctgaaggtgccaacctcaattcatccaacagcctgggcgtcagtgccctgtgcgccatctgtggcgaccgggccaccggcaaacactacggagcctcgagctgtgacggctgcaaggggttcttcaggaggagcgtgaggaagaaccacatgtactcctgcaggtttagccgacaatgtgtggtagacaaagataagaggaaccagtgtcgttactgcaggcttaagaagtgcttccgggctggcatgaagaaggaagctgtccaaaatgagcgggaccggatcagcacgcggaggtcaagctacgaggacagcagcctgccctccatcaacgcgctcctgcaggcagaggttctgtcccagcagatcacctctcccatctctgggatcaatggcgacattcgggcaaagaagattgccaacatcacagacgtgtgtgagtctatgaaggagcagctgctggtcctggtcgagtgggccaagtacatcccggccttctgcgaactccttctggatgaccaggtggcgctgctcagggcccacgccggtgagcatctgctgcttggagccaccaagaggtccatggtgtttaaggacgtgctgctcctaggcaatgactacatcgtccctcggcactgtccagagctagcggagatgagccgtgtgtccatccgcatcctcgatgagctggtcctgcccttccaagagctgcagattgatgacaatgaatatgcctgcctcaaagccatcatcttctttgatccagatgccaaggggctgagtgacccgggcaagatcaagcggctgcggtcacaggtgcaagtgagcctggaggattacatcaacgaccggcagtacgactctcggggccgctttggagagctgctgctgctgttgcccacgctgcagagcatcacctggcagatgatcgaacagatccagttcatcaagctcttcggcatggccaagattgacaacctgctgcaggagatgcttctcggagggtctgccagtgatgcaccccacacccaccaccccctgcaccctcacctgatgcaagaacacatgggcaccaatgtcattgttgctaacacgatgccctctcacctcagcaatggacagatgtgtgagtggccccgacccagggggcaggcagccactcccgagactccacagccatcaccaccaagtggctcgggatctgaatcctacaagctcctgccaggagccatcaccaccatcgtcaagcctccctctgccattccccagccaacgatcaccaagcaagaagccatctaggctggagcctcggtggccatgcttcttgccccttgggcctccccccagcccctcctccccttcctgcacccgtacccccgtggtctttgaataaagtctgagtgggcggc

SEQ ID NO: 10 - RNA, 1687 nt, synthetic construct
  source 1..1687: mol_type = other RNA
ttaatacgactcactataagggagacccaagctggctagcgtttaaacttaagcttgaataaactagtattcttctggtccccacagactcagagagaacccgccaccaagggagactcttctggtccccacagactcagagagaacccaccatgcgactctctaaaacccttgccggcatggatatggccgactacagcgctgccctggacccagcctacaccaccctggagtttgaaaatgtgcaggtgttgaccatgggcaatgacacgtccccatctgaaggtgccaacctcaattcatccaacagcctgggcgtcagtgccctgtgcgccatctgtggcgaccgggccaccggcaaacactacggagcctcgagctgtgacggctgcaaggggttcttcaggaggagcgtgaggaagaaccacatgtactcctgcaggtttagccgacaatgtgtggtagacaaagataagaggaaccagtgtcgttactgcaggcttaagaagtgcttccgggctggcatgaagaaggaagctgtccaaaatgagcgggaccggatcagcacgcggaggtcaagctacgaggacagcagcctgccctccatcaacgcgctcctgcaggcagaggttctgtcccagcagatcacctctcccatctctgggatcaatggcgacattcgggcaaagaagattgccaacatcacagacgtgtgtgagtctatgaaggagcagctgctggtcctggtcgagtgggccaagtacatcccggccttctgcgaactccttctggatgaccaggtggcgctgctcagggcccacgccggtgagcatctgctgcttggagccaccaagaggtccatggtgtttaaggacgtgctgctcctaggcaatgactacatcgtccctcggcactgtccagagctagcggagatgagccgtgtgtccatccgcatcctcgatgagctggtcctgcccttccaagagctgcagattgatgacaatgaatatgcctgcctcaaagccatcatcttctttgatccagatgccaaggggctgagtgacccgggcaagatcaagcggctgcggtcacaggtgcaagtgagcctggaggattacatcaacgaccggcagtacgactctcggggccgctttggagagctgctgctgctgttgcccacgctgcagagcatcacctggcagatgatcgaacagatccagttcatcaagctcttcggcatggccaagattgacaacctgctgcaggagatgcttctcggagggtctgccagtgatgcaccccacacccaccaccccctgcaccctcacctgatgcaagaacacatgggcaccaatgtcattgttgctaacacgatgccctctcacctcagcaatggacagatgtgtgagtggccccgacccagggggcaggcagccactcccgagactccacagccatcaccaccaagtggctcgggatctgaatcctacaagctcctgccaggagccatcaccaccatcgtcaagcctccctctgccattccccagccaacgatcaccaagcaagaagccatctaggctggagcctcggtggccatgcttcttgccccttgggcctccccccagcccctcctccccttcctgcacccgtacccccgtggtctttgaataaagtctgagtgggcggc

SEQ ID NO: 11 - DNA, 1656 bp, synthetic construct
  source 1..1656: mol_type = other DNA
taatacgactcactataagggagacccaagctggctagcgtttaaacttaagcttgaataaactagtattcttctggtccccacagactcagagagaacccgccaccaagggagactcttctggtccccacagactcagagagaacccaccatgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatctaggctggagcctcggtggccatgcttcttgccccttgggcctccccccagcccctcctccccttcctgcacccgtacccccgtggtctttgaataaagtctgagtgggcggc

SEQ ID NO: 12 - RNA, 1656 nt, synthetic construct
  source 1..1656: mol_type = other RNA
taatacgactcactataagggagacccaagctggctagcgtttaaacttaagcttgaataaactagtattcttctggtccccacagactcagagagaacccgccaccaagggagactcttctggtccccacagactcagagagaacccaccatgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatctaggctggagcctcggtggccatgcttcttgccccttgggcctccccccagcccctcctccccttcctgcacccgtacccccgtggtctttgaataaagtctgagtgggcggc

SEQ ID NO: 13 - DNA, 24 bp, synthetic construct
  source 1..24: mol_type = other DNA
tgttcgccactgtatccagaagtc

SEQ ID NO: 14 - DNA, 23 bp, synthetic construct
  source 1..23: mol_type = other DNA
ccgctcctctttgtcagggtcta

SEQ ID NO: 15 - DNA, 21 bp, synthetic construct
  source 1..21: mol_type = other DNA
acactgtttgcctagaaagcc

SEQ ID NO: 16 - DNA, 21 bp, synthetic construct
  source 1..21: mol_type = other DNA
ccatgacagccatgattgtcc

SEQ ID NO: 17 - DNA, 23 bp, synthetic construct
  source 1..23: mol_type = other DNA
cggactcctgatgaccctcaagt

SEQ ID NO: 18 - DNA, 24 bp, synthetic construct
  source 1..24: mol_type = other DNA
tgtactgttccacgctgtgactgt

SEQ ID NO: 19 - DNA, 22 bp, synthetic construct
  source 1..22: mol_type = other DNA
acatggtgaccaagattcctcg

SEQ ID NO: 20 - DNA, 21 bp, synthetic construct
  source 1..21: mol_type = other DNA
ttcctcaaaggtgcgaccaat

SEQ ID NO: 21 - DNA, 21 bp, synthetic construct
  source 1..21: mol_type = other DNA
agatagcggccagtcagaata

SEQ ID NO: 22 - DNA, 23 bp, synthetic construct
  source 1..23: mol_type = other DNA
ctcagttcatagccaccaaagaa

SEQ ID NO: 23 - DNA, 21 bp, synthetic construct
  source 1..21: mol_type = other DNA
tgtccctaatgacagctcctt

SEQ ID NO: 24 - DNA, 21 bp, synthetic construct
  source 1..21: mol_type = other DNA
gcatccacccaaatgacacat

SEQ ID NO: 25 - DNA, 23 bp, synthetic construct
  source 1..23: mol_type = other DNA
aacacccagatgcaaaactttca

SEQ ID NO: 26 - DNA, 22 bp, synthetic construct
  source 1..22: mol_type = other DNA
gacttggagttagctgctcttt

SEQ ID NO: 27 - DNA, 19 bp, synthetic construct
  source 1..19: mol_type = other DNA
cacgcggaggtcaagctac

SEQ ID NO: 28 - DNA, 21 bp, synthetic construct
  source 1..21: mol_type = other DNA
cccagagatgggagaggtgat

SEQ ID NO: 29 - DNA, 21 bp, synthetic construct
  source 1..21: mol_type = other DNA
aaaacacgggacgagaaaccc

SEQ ID NO: 30 - DNA, 22 bp, synthetic construct
  source 1..22: mol_type = other DNA
acgggaacctttattaacccct

SEQ ID NO: 31 - DNA, 21 bp, synthetic construct
  source 1..21: mol_type = other DNA
tgctttttccaggggtgtgtt

SEQ ID NO: 32 - DNA, 23 bp, synthetic construct
  source 1..23: mol_type = other DNA
ttacttcctgcactaatttggca

SEQ ID NO: 33 - DNA, 24 bp, synthetic construct
  source 1..24: mol_type = other DNA
tctggtagattgtcgcttatcttg

SEQ ID NO: 34 - DNA, 28 bp, synthetic construct
  source 1..28: mol_type = other DNA
tagatgctgttactgataggaaatcgag

SEQ ID NO: 35 - DNA, 23 bp, synthetic construct
  source 1..23: mol_type = other DNA
aatctaccatcctccgtgaaacc

SEQ ID NO: 36 - DNA, 25 bp, synthetic construct
  source 1..25: mol_type = other DNA
tcagtttagctacccccaagtttaa

SEQ ID NO: 37 - DNA, 18 bp, synthetic construct
  source 1..18: mol_type = other DNA
aacggatccacagccgta

SEQ ID NO: 38 - DNA, 18 bp, synthetic construct
  source 1..18: mol_type = other DNA
agtcctcgggccatgatt

SEQ ID NO: 39 - DNA, 25 bp, synthetic construct
  source 1..25: mol_type = other DNA
gctttccacttcatcttaccattta

SEQ ID NO: 40 - DNA, 20 bp, synthetic construct
  source 1..20: mol_type = other DNA
tgttgggttgtttgatcctg

SEQ ID NO: 41 - DNA, 25 bp, synthetic construct
  source 1..25: mol_type = other DNA
ctagctcatgtgtcaagaccctctt

SEQ ID NO: 42 - DNA, 20 bp, synthetic construct
  source 1..20: mol_type = other DNA
gccagcacgtttctctcgtt

SEQ ID NO: 43 - DNA, 18 bp, synthetic construct
  source 1..18: mol_type = other DNA
cagggccatctcagaacc

SEQ ID NO: 44 - DNA, 20 bp, synthetic construct
  source 1..20: mol_type = other DNA
gctattggaaagcctcttgc

SEQ ID NO: 45 - RNA, 22 nt, Mus musculus
  source 1..22: mol_type = transcribed RNA
tggctcagttcagcaggaacag

SEQ ID NO: 46 - RNA, 17 nt, synthetic construct
  source 1..17: mol_type = other RNA
taatacgactcactata

SEQ ID NO: 47 - RNA, 52 nt, synthetic construct
  source 1..52: mol_type = other RNA
gaataaactagtattcttctggtccccacagactcagagagaacccgccacc

(19) United States
(12) Patent Application Publication
(10) Pub. No.: US 2025/0057985 A1
(43) Pub. Date: Feb. 20, 2025

(54) REEXPRESSION OF HNF4A TO ALLEVIATE CANCER-ASSOCIATED CACHEXIA

(71) Applicants: YEDA RESEARCH AND DEVELOPMENT CO. LTD., Rehovot (IL); TECHNION RESEARCH & DEVELOPMENT FOUNDATION LIMITED, Haifa (IL)

(72) Inventors: Ayelet EREZ, Moshav Bnei Zion (IL); Omer GOLDMAN, Rehovot (IL); Emma HAJAJ, Rehovot (IL); Avi SCHROEDER, Haifa (IL)

(21) Appl. No.: 18/933,233

(22) Filed: Oct. 31, 2024

Related U.S. Application Data
(63) Continuation of application No. PCT/IL2023/050443, filed on May 1, 2023.
(60) Provisional application No. 63/337,113, filed on May 1, 2022.
(60) Provisional application No. 63/440,723, filed on Jan. 24, 2023.

(51) Int. Cl. A61K 48/00; A61K 38/17; A61P 35/00; C07K 16/24

(52) U.S. Cl. CPC A61K 48/0058; A61K 38/177; A61K 48/0033; A61P 35/00; C07K 16/248

(57) ABSTRACT

Methods of predicting clinical outcome in a subject suffering from cancer, detecting non-liver cancer in a subject, and methods of treating or preventing cancer or cancer-associated cachexia are provided. Synthetic lipid nanoparticles encapsulating an mRNA encoding for HNF4A and composition comprising same are also provided.

### CROSS REFERENCE TO RELATED APPLICATIONS

This application is a ByPass Continuation of PCT Patent Application No. PCT/IL2023/050443 having International filing date of May 1, 2023, which claims the benefit of priority of U.S. Provisional Patent Application No. 63/337,113 filed on May 1, 2022, and U.S. Provisional Patent Application No. 63/440,723 filed on Jan. 24, 2023 both titled “REEXPRESSION OF HNF4A TO ALLEVIATE CANCER-ASSOCIATED CACHEXIA”, the contents of which are all incorporated herein by reference in their entirety.

### REFERENCE TO AN ELECTRONIC SEQUENCE LISTING

The contents of the electronic sequence listing (YEDA-TECH-P-025-PCT.xml; Size: 55,530 bytes; and Date of Creation: Apr. 30, 2023) is herein incorporated by reference in its entirety.

### FIELD OF INVENTION

The present invention is in the field of cancer and cancer associated cachexia treatment.

### BACKGROUND OF THE INVENTION

The liver communicates with all the organs in our body and with the tumor, either directly via messenger molecules or indirectly via the immune system. Notably, the liver is also an immunogenic organ containing diverse resident immune cells that can respond to systemic or tissue-specific immune-related vulnerabilities by producing acute-phase proteins, complement components, cytokines, and chemokines. While aiming to be protective, recent work demonstrates that the immune system can also exert selective pressures that promote cancerous features in normal tissue-resident cells. Thus, established hallmarks of carcinogenesis, such as systemic inflammation, the tumor microenvironment, and the host, may all promote carcinogenesis.

Unrestricted cancer growth requires a permanent supply of glucose, amino, and fatty acids. These nutrient demands are obtained from cancer-mediated reprogramming of metabolism in the tumor and its microenvironment. Unraveling these interactions led to synergistic therapy combining chemotherapy with drugs targeting metabolic dependencies between the tumor and the microenvironment. In addition to the tumor microenvironment, the tumor connects with the host with networks of nerves, blood, and lymph vessels, and extends its effect from the microenvironment to external organs, such as the liver. Consequently, the tumor can dysregulate tissue-specific metabolism and induces systemic metabolic rewiring, potentially contributing to cancer manifestations.

Because the liver is a central metabolic organ essential for maintaining body homeostasis, it senses and responds to systemic nutrient-level fluctuations by facilitating tissue-specific adaptations that preserve systemic equilibrium. At the cellular level, hepatocytes play significant roles in carbohydrate, protein, amino acid, and lipid metabolism. Some of these metabolic reactions are mostly liver-specific such as the complete urea cycle (UC), which disposes of excess nitrogen in the form of ammonia by converting it to urea. Decreased UC activity in the livers of 4T1 breast-cancer-bearing mice and plasma of children with cancer has been reported, supporting a potential metabolic communication between extrahepatic tumors and the liver.

The primary metabolic phenomenon accompanying cancer is cancer-associated cachexia (CAC). CAC occurs in 80% of cancer patients at late disease stages, manifesting as weight loss, skeletal muscle wasting, and atrophy of the adipose tissue, estimated to be the direct cause of clinical deterioration that leads to death in at least 20% of cancer patients. Specifically, CAC is highly prevalent in pancreatic cancer, affecting more than 70% of patients. CAC is divided into three consecutive clinical stages: pre-cachexia, cachexia, and refractory cachexia. Pre-cachexia is defined as less than 5% of body weight loss, while the patient loses more than 5% of his body weight in cachexia. The refractory cachexia phase is determined by a low WHO performance status score and a survival period of less than 3 months. At present, there are no specific biomarkers for pre-cachexia identification and thus, most patients are diagnosed at the cachectic or at the incurable refractory cachexia stages. Diagnostic methods of determining metabolic changes in cancer and the onset of cachexia as well as therapies that target these metabolic changes as well as prevent cachexia progression are greatly needed.

### SUMMARY OF THE INVENTION

The present invention provides methods of predicting clinical outcome in a subject suffering from cancer, detecting non-liver cancer in a subject, and methods of treating or preventing cancer or cancer-associated cachexia. Synthetic lipid nanoparticles encapsulating an mRNA encoding for HNF4A and composition comprising same are also provided.

According to a first aspect, there is provided a method of predicting a clinical outcome in a subject suffering from cancer, wherein the cancer is a non-hepatic cancer, the method comprising measuring function of the urea cycle in the subject, wherein decreased urea cycle function as compared to urea cycle function in a healthy control indicates a worse clinical outcome as compared to a subject without decreased urea cycle function, thereby predicting a clinical outcome in a subject.

According to some embodiments, the non-hepatic cancer is selected from breast cancer and pancreatic cancer, does not comprise detectable metastasis to the liver or both.

According to some embodiments, measuring function of the urea cycle comprises at least one of:
a. measuring in the liver of the subject expression of at least one urea cycle enzyme selected from: argininosuccinate synthetase 1 (ASS1), ornithine transcarbamoylase (OTC), argininosuccinate lyase (ASL), carbamoyl phosphate synthetase-1 (CPS1) and ornithine translocase (ORNT1/SLC25A15);
b. measuring urea to glutamine ratio or urea to glutamate ratio in the blood or liver of the subject;
c. measuring glutamate, aspartate or fumarate levels in the blood or liver of the subject;
d. measuring ammonia levels in the blood of the subject;
e. measuring urea levels in the urine of the subject;
f. measuring hepatocyte nuclear factor 4 alpha (HNF4A) expression in the liver of the subject;
g. measuring in the blood of the subject levels of at least one liver enzyme selected from: aspartate aminotransferase (AST), alanine aminotransferase (ALT), alkaline phosphatase (ALK-P/ALP), and lactate dehydrogenase (LDH);
h. measuring the prothrombin time international normalized ratio (INR) of blood from the subject; and
i. measuring albumin in the blood or liver of the subject.

According to some embodiments,
a. a decrease in expression of the at least one urea cycle enzyme;
b. a decrease in urea to glutamine ratio or urea to glutamate ratio;
c. an increase in glutamate or aspartate;
d. a decrease in fumarate;
e. an increase in ammonia;
f. a decrease in urea;
g. a decrease in HNF4A;
h. an increase in levels of the at least one liver enzyme;
i. a decrease in INR;
j. a decrease in albumin; or
k. any combination thereof
is indicative of decreased urea cycle function in the subject.

According to some embodiments, the measuring function comprises producing a liver-function score and wherein a liver-function score beyond a predetermined threshold indicates decreased urea cycle function.

According to some embodiments, the liver-function score is a weighted sum of normalized levels of AST, ALT, ALP, and albumin and INR in a blood sample from the subject.

According to some embodiments, the score is standardized from 0 to 1, the predetermined threshold is 0.6 and wherein a score above the predetermined threshold indicates decreased urea cycle function.

According to some embodiments, the clinical outcome is development of cancer-associated cachexia and wherein decreased urea cycle function is predictive of an increased risk of developing cancer-associated cachexia.

According to some embodiments, the clinical outcome is overall survival and wherein liver-function score beyond a predetermined threshold indicates a reduced overall survival time.

According to another aspect, there is provided a method of detecting a non-hepatic cancer in a subject in need thereof, the method comprising receiving a blood sample from the subject and measuring function of the urea cycle in the subject based on the blood sample, wherein decreased urea cycle function as compared to urea cycle function in a healthy control indicates the subject suffers from a non-liver cancer thereby detecting a non-hepatic cancer in the subject.

According to some embodiments, the non-hepatic cancer is selected from breast cancer and pancreatic cancer, does not comprise detectable metastasis to the liver or both.

According to some embodiments, measuring function of the urea cycle comprises at least one of:
a. measuring glutamine to glutamate ratio in the blood sample;
b. measuring glutamate, aspartate or fumarate levels in the blood sample;
c. measuring ammonia levels in the blood sample;
d. measuring aspartate aminotransferase (AST) level in the blood sample; and
e. measuring albumin levels in the blood sample.

According to some embodiments,
a. a decrease in glutamine to glutamate ratio;
b. an increase in glutamate or aspartate;
c. a decrease in fumarate;
d. an increase in ammonia;
e. an increase in AST level;
f. a decrease in albumin level; or
g. any combination thereof
is indicative of decreased urea cycle function in the subject.

According to some embodiments, the method further comprises administering to a subject with a worse clinical outcome or determined to have non-hepatic cancer at least one therapeutic agent selected from: an anti-IL6 blocking antibody, an ERK inhibitor, a STAT3 inhibitor and an agent capable of increasing expression of HNF4A in a liver of the subject.

According to another aspect, there is provided a synthetic lipid nanoparticle (LNP) comprising encapsulated therein an mRNA encoding for HNF4A, wherein:
a. the lipid nanoparticle comprises SM-102 lipid, cholesterol, DOPE, and DMG-PEG;
b. the mRNA comprises or consists of a sequence of SEQ ID NO: 10 or 12 or a sequence comprising at least 85% identity thereto and encoding HNF4A and a poly-A tail; or
c. both.

According to some embodiments, the mRNA comprises a 5′ cap and a poly-A tail.

According to some embodiments, the mRNA encoding for HNF4A comprises the mRNA coding sequence of SEQ ID NO: 2 or SEQ ID NO: 5 or a sequence with at least 85% identity thereto which encodes for HNF4A.

According to some embodiments, the lipid nanoparticle targets to liver cells.

According to some embodiments, the lipid nanoparticle comprises about 50 mol % SM-102, 38.5 mol % cholesterol, 10 mol % DOPE, and 1.5 mol % DMG-PEG200.

According to another aspect, there is provided a pharmaceutical composition comprising the synthetic LNP of the invention and a pharmaceutically acceptable carrier excipient or adjuvant.

According to another aspect, there is provided a method of treating a non-hepatic cancer in a subject in need thereof, the method comprising administering to the subject an agent capable of increasing expression of HNF4A in a liver of the subject, thereby treating the non-hepatic cancer in a subject.

According to another aspect, there is provided a method of treating or preventing cancer-associated cachexia in a subject in need thereof, the method comprising administering to the subject a composition comprising at least one agent selected from: an anti-IL6 blocking antibody, an ERK inhibitor, a STAT3 inhibitor and an agent capable of increasing expression of HNF4A in a liver of the subject, thereby treating of preventing cancer-associate cachexia in a subject.

According to some embodiments, the agent comprises a nucleic acid molecule encoding the HNF4A.

According to some embodiments, the nucleic acid molecule is contained within an adeno-associated virus (AAV).

According to some embodiments, the nucleic acid molecule is an mRNA.

According to some embodiments, the mRNA comprises a 5′ cap and a poly-A tail.

According to some embodiments, the nucleic acid molecule comprises or consists of SEQ ID NO: 10 or 12 or comprising at least 85% identity thereto and encoding HNF4A.

According to some embodiments, the agent is a synthetic LNP of the invention.

According to some embodiments, the subject suffers from early-stage cancer, a pre-cancerous lesion or is at risk of developing cancer.

According to some embodiments, the subject is determined to have the non-hepatic cancer by a method of the invention.

According to some embodiments, the subject is determined to have an increased risk of developing the cancer-associated cachexia by a method of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-L: Breast cancers induce metabolic changes in the liver during early carcinogenesis. (1A) RT-PCR of livers from breast cancer (BC)-bearing mice demonstrates decreased RNA expression of UC enzymes along cancer progression (n=5, student T-Test). Day 4, P=0.003, Day 14, P=0.033, 0.01, 0.028 (respectively), Day 21, P=0.0002, 0.019, 0.0002, 0.013, 0.007 (respectively). (1B) Left panel—Western-blots demonstrating decreased protein expression levels of ASS1 and OTC in livers of BC-bearing mice vs. WT-PBS injected mice (n=5, student T-Test). Right panel—quantification of band intensities, ASS1, P=0.002. OTC, P=0.011. (1C) RT-PCR of liver from MMTV-PyMT of 14 weeks old cancer-bearing mice demonstrate decreased RNA expression of UC enzymes compared to livers of WT mice (WT n=5, MMTV-PyMT n=3), P=0.033, 3.14E-05, 0.038 (respectively). (1D) Measurements of UC-related metabolites following Glutamine ¹⁵N₂ infusion to 4T1 BC bearing or PBS injected mice in the plasma and livers. Left panels: A decreased ratio of urea to glutamine and glutamate m+1 isotopologues in the plasma of 4T1 BC bearing mice (WT n=5, 4T1 n=7, student T-Test) P=0.019, 0.042 respectively. Right panels: An increase in the levels of glutamate (AUC/Internal standard/dry weight) in 4T1 BC-bearing mice and a decrease in the ratio of urea to glutamate following infusion in 4T1 BC-bearing mice, supporting a malfunctioning UC (n=7, student T-Test) P=0.016, 0.048. (1E) Elevated levels of aspartate, glutamate, and decreased levels of fumarate, in livers (Upper panel) and plasma (Lower panel) of BC-bearing mice compared to livers of WT-PBS injected mice. UC intermediate levels were measured with gas chromatography-mass spectrometry (GC-MS), amino acid analyzer (AAA), and liquid chromatography-mass spectrometry (LC-MS) (GC-MS, n=5, AAA WT n=5, 4T1 n=4, LCMS n=5, student T-Test), P values for Liver: Asp=0.027, Glu=0.049, Fum=0.045. Pvalues for Plasma: Asp=0.004, Glu=0.0005. (1F) Left panel—Measurements of ammonia levels, using an ammonia assay kit, in the plasma of BC-bearing mice compared to WT-PBS injected mice (WT n=4, an outlier—IQR method, 4T1 n=5, student T-Test). Right panel—Urea levels in the urine of BC-bearing mice compared to WT-PBS injected mice. P=0.033. (1G) Left panel: Decreased levels of m+1 labeled aspartate in the tumor of 4T1 BC-bearing mice compared to the levels in the plasma and liver following infusion of ¹⁵N₂ glutamine (n=8, plasma n=7, 2 way ANOVA) P values: plasma vs. tumor=0.041, liver vs. tumor=0.007. Right panel: Increased ratio of m+1 uracil to aspartate m+1 isotopologues in the tumors of 4T1 BC-bearing mice (n=8, plasma n=7, 2 way ANOVA) P values: plasma vs. tumor=0.042, liver vs. tumor=0.008. (1H) XTT assay for 4T1 cancer cells' proliferation following supplementation of the medium with UC intermediates (n=3, two-way ANOVA) P values: 24 h: Asp-ns, Amn=0.007, Glu and Fum <0.0001.48 and 72<0.0001 for all measurements. Metabolites concentrations were supplemented in the following concentrations: ammonia—0.75 mM as published [49], aspartate 0.25 mM, fumarate 0.35 mM, glutamine 0.25 mM. (1I) Ex-vivo FACS analysis of CD8+ splenocytes cells' survival (Left) and activation (Right) following supplementation with 1 mM ammonia [58](measured plasma ammonia levels 1.34 mM), (n=5, student T-Test) P=0.011, 0.043, respectively. (1J) A Heatmap for differential gene expression in hepatocytes demonstrates a unique pattern on day 21 in the livers of 4T1 BC mice compared to day 4. (1K) 4T1 d21 vs. 4T1 d4 pathways enrichment analysis. Each bar shows the fold enrichment of a specific pathway. (1L) Left panel-plasma measurements of AST demonstrate significantly increased levels in BC-bearing mice in comparison to WT-PBS injected mice (n=5, student T-Test) P=0.011. Right panel—no significant changes were found for ALT.

FIGS. 2A-I: Innate immune cells infiltrate the host's liver during early breast carcinogenesis. (2A) H&E staining of liver sections demonstrates increased immune cells' infiltration along a time course following BC cells' injection. Magnifications—10 and 40× as detailed on the figure. (2B) Cell types annotated from the single-cell RNA sequencing analysis are projected on 3 UMAPs, indicating which cells appear at which time point. The arrows mark the neutrophils and monocytes subsets that were absent in the liver of WT mice and gradually accumulated in the liver until day 21. (2C-E) CyTOF-tSNE analysis (2C, 2E) and (2D) quantification of liver and blood CD45+ populations show increased levels of innate immune cells and reduction of lymphocytes cells in the livers (2C) and blood (2E) of BC mice compared to WT-PBS injected mice (WT n=3, 4T1=5, student T-Test). P values for blood <0.0001. P values for Liver: B cells=0.013, T cells=0.0014, Neutrophils=0.0001. (2F) Multiplex ELISA immunoassay demonstrates increasing levels of IL-6 (Left) and TNFα levels (Right) in BC-bearing mice from day 4 to day 21 following cancer cells' injection (n=5, student T-Test). P values for IL-6: day 4=0.024, day 14<0.0001, day 21=0.0009. P values for TNFα: day 4=0.05, day 14 and 21<0.0001 (2G) ELISA assay measurements of CCL2 levels in plasma, livers, spleens, and lungs of BC-bearing mice (n=5, student T-Test) P values: Plasma=0.0005, Liver=0.017, Spleen=0.04. (2H) ELISA assay demonstrates a significant interaction effect between the time following cancer cells injection and elevation of CCL2 in the liver of BC mice relative to WT-PBS injected mice (n=5, 2-way ANOVA) P=0.0034. (2I) the expression of CCR2 in immune cells from scRNAseq experiment, projected on the UMAP demonstrates that CCR2 is mainly expressed in monocyte subpopulations.

FIGS. 3A-I: Activated liver infiltrating myeloid cells perturb liver metabolism via HNF4α depletion. (3A) DE genes pathway enrichment analysis of hepatocytes from BC-bearing mice on day 21 compared to day 4. The bar shows the Z score of a specific pathway. (3B) Immunofluorescence staining demonstrates increased levels of pERK in CD45+ cells in the livers of BC-bearing mice. (3C) Mean Fluorescence Intensity (MFI) of pERK in CD45+ cells demonstrates a specific increase for pERK in the livers of BC mice compared to WT-PBS injected mice (Left panel), and no significant difference in the staining for CD45+ pERK in the blood (Middle panel) (n=4, student T-Test). Quantification is shown on the Right panel. P<0.0001. (3D) Normalized RNA Seq counts for 3 integrins-binders expressed on hepatocytes on day 4, and on day 21. P<0.0001, 0.001, 0.027 (respectively). (3E) Normalized RNA expression counts of integrins (Left panel—Itga4, Middle panel—Itgal, Right panel—Itgam) in neutrophils, monocytes, macrophages, and hepatocytes. (3F) Western blots demonstrating increased protein expression levels of pSTAT3 and decreased expression of HNF4α in livers of BC-bearing mice compared to livers from WT-PBS injected mice (n=5, student T-Test). (3G) RT-PCR of livers from BC-bearing mice demonstrates an increase in miR-24 levels (n=5, student T-Test) P=0.005. (3H) RT-PCR of livers from BC-bearing mice demonstrates a significant decrease in HNF4α levels (n=5, student T-Test) P=0.005. (3I) RT-PCR of primary hepatocytes demonstrates increased RNA expression of CCL2 following supplementation of IL-6 (n=3, student T-Test, representative experiment of two independent biological replicates) P=0.009.

FIGS. 4A-J: IL-6-STAT3-HNF4α signaling causes changes in liver metabolism during BC carcinogenesis. (4A) DE pathway enrichment analysis for genes regulated by HNF4α, in livers of BC mice on day 21 compared to day 4. The bar shows the Z score of a specific pathway. (4B). Left panel—Plasma albumin measurements demonstrate decreased levels in BC-bearing mice compared to WT-PBS injected mice (n=5, student T-Test) P=0.002. Right panel—Normalized RNA Seq analysis of albumin expression in hepatocytes on day 4 and day 21. P=0.013. (4C). RT-PCR of primary hepatocytes demonstrates decreased RNA expression levels of OTC following IL-6 supplementation (n=3, student T-Test, representative experiment of two independent biological replicates) P=0.0002. (4D) STAT3 inhibitor HJCO152 restores OTC levels even in the presence of IL-6 (n=3, student T-Test) P<0.0001 for CTRL+DMSO vs IL-6+DMSO and STAT3 inhibitor vs STAT3 inhibitor+IL-6. (4E) Western blots (Left panel) demonstrate increased expression levels of HNF4α in BC mice treated with an ERK inhibitor compared to livers from BC mice treated with a vehicle (Vehicle n=4, Trametinib n=3, student T-Test). Right panel quantification of band intensities. P=0.024. (4F) Measurement of tumor weight on days 8 and 14 following treatment with an ERK inhibitor (Day 8 n=5, day 14 n=4, student T-Test) P values: Day 8=0.007, Day 14=0.014. (4G) RT-PCR of livers from BC-bearing mice treated with AAV8-HNF4α demonstrates increased RNA expression of HNF4α, UC enzymes, and albumin in comparison to AAV8-GFP treated BC-bearing mice (AAV8-GFP=4, AAV8-HNF4α n=7, student T-Test) P=0.004, 0.004, 0.002, 0.001, respectively. (4H) 4T1 tumor growth is significantly higher following AAV-GFP injection compared to AAV-HNF4α (AAV8-GFP=12, AAV8-HNF4α n=13, student T-Test) P=0.049. (4I) Left panel—Western blots showing decreased levels of PCNA in tumors from BC-bearing mice treated with HNF4α compared to mice treated with GFP. Right panel—quantification of band intensity relative to actin. P=0.003. (4J) RT-PCR of livers and tumors from BC-bearing mice treated with AAV8-HNF4α demonstrates a significant increase in the RNA expression of viralHNF4α (BGH polyadenylation signal) in the liver (n=7, Student T-Test) P<0.0001.

FIGS. 5A-M: Innate immune cells induce similar transcriptional and metabolic changes in the livers of KPC mice. (5A) RT-PCR of livers from PC-bearing mice demonstrate a decreased RNA expression of OTC enzyme in the first week following cancer injection (n=6, 2-way ANOVA) P=0.01. (5B) Elevated levels of aspartate and glutamate and decreased levels of fumarate in the livers of PC-bearing mice. UC intermediate levels were measured with GC-MS, and amino acids analyzer (AAA) (n=4, glutamate—an outlier—IQR method. Fumarate—WT n=5, KPC n=7, student T-Test) P values: Asp=0.001, Glu=0.019, Fum=0.0001. (5C) H&E staining of liver sections demonstrates increased immune cell infiltration following the injection of PC cells. Magnification 10× as detailed in the figure. (5D) Left panel—FACS analysis quantification of liver and blood CD45+ populations shows increased levels of innate immune cells and reduction of lymphocytes cells in the livers and blood of PC-bearing mice compared to WT-PBS injected mice (n=8, liver WT=7, student T-Test) P values: Blood: B cells=0.003, monocytes=0.0002, neutrophils=0.006. Liver: B cells=0.004, T cells=0.05, monocytes <0.0001, neutrophils=0.004. Right panel—MFI=0.0001. Live immune cells in the blood and livers of KPC PC-bearing mice were gated asCD45+, CD11b, CD3, CD19 Ly-6G, and Ly-6C. Mean fluorescence intensity was done onLy-6C high monocytes. (5E) ELISA assay demonstrates increased levels of IL-6 in PC-bearing mice (n=5, student T-Test) P=0.011, 0.01, respectively. (5F) Western blots demonstrate increased protein expression levels of pSTAT3 in PC bearing mice livers compared to WT-PBS injected mice (n=6). (5G) RT-PCR of livers from pancreatic cancer bearing mice demonstrates a significant decrease in HNF4α RNA expression levels with tumor progression while remaining constant in WT-PBS injected mice (WT n=3, KPC n=4, student T-Test) P=0.0012. (5H) RT-PCR of livers from PC-bearing mice demonstrate a significant decrease in albumin RNA levels (WT n=4, KPC n=5, student T-Test), P=0.0009. (5I) Treatment of PC mice with IL-6 antibodies preserves plasma levels of albumin (Ctrl IgG=8, IL-6 Ab=5, student T-Test) P=0.011. (5J) Weight measurements of PC mice along 22 days of tumor development show no weight loss in PC mice compared to WT control. (5K) Power analysis of the in vivo NMR experiment. Y-axis shows the Wilcoxon rank-sum P-value for the weight change differences between HNF4α—injected vs. control GFP injected mice, and the X-axis shows the sample size per each condition in each experiment considered for the statistical test. In this experiment, we have two conditions (HNF4α-injected vs. control GFPinjected) and 2 repeated experiments, so the total number of experiments is 4 times of the numbers shown in the Xaxis, and the mean sample size is 7. For each sample size of less than 7, the mice were randomly selected for the statistical test, and each box shows the mean and standard deviation of the P-values, and the wisker shows the top and bottom 10 percentile of the P-values. (5L) Weight measurements and nuclear magnetic resonance (NMR) body composition analysis of PC AAV-HNF4α injected mice demonstrate significantly less weight loss (Left panel), increased fat tissue (Middle panel), and decreased free fluids (Right panel) compared to AAV-GFP injected mice (relative to day 0); (n=7). (5M) Laminin immunofluorescence staining was performed on Gastrocnemius sections from KPC-AAV-HNF4α and KPC-AAV-GFP injected mice 4 weeks following cancer cell injection. For each slide, five pictures were taken from different areas along the tissue. Fiji-ImageJ was used to calculate the fiber area (μm²). The mean area of the five pictures was used to calculate the fiber mean area per mouse. A lower cutoff of 200 μm² was used to eliminate artifacts. (AAV-GFP n=9, AAV-HNF4A n=10, 2-way ANOVA). P=0.008.

FIGS. 6A-J: CCR2 KO mice and HNF4α rescue the early changes in liver metabolism in PC mice. (6A) H&E staining of livers from CCR2−/− PC and PC bearing mice demonstrate decreased immune cell infiltration to the livers of CCR2−/− compared to livers of WT PC mice. PC mice are mice injected with KPC. Magnifications—10, 20 and 40× as detailed in the figure. (6B) ELISA assay demonstrate decreased IL-6 levels in the plasma of PC-CCR2−/− bearing mice relative to PC-WT mice (PC WT n=7, CCR2−/− n=6, Student T-Test) P=0.0125. (6C) RT-PCR of livers from CCR2−/− PC-bearing mice demonstrates preserved RNA expression levels of ASS1, OTC, Albumin, and HNF4α compared to decreased expression levels of these genes in livers of WT PC (PC WT n=5, CCR2−/− n=6, Student T-Test, student T-Test) P=0.019, 0.011, 0.002 respectively. (6D) Decreased levels of aspartate and glutamate in the liver of PC-CCR2−/− bearing mice. UC intermediate levels were measured with gas chromatography-mass spectrometry (GC-MS) (n=7, Student T-Test) P=0.011, 0.029, respectively. (6E) Weight measurements and nuclear magnetic resonance (NMR) body composition analysis of CCR2−/− PC-bearing mice demonstrate significantly less weight loss (Left panel), increased fat tissue (Middle panel), and decreased free fluids (Right panel) in comparison to WT PC on day 21 (relative to day 0); (n=7 in each group, Student T-test) P=0.014, 0.006, 0.004, respectively. (6F) Tumor weights measured on the day of sacrifice are significantly lower in the CCR2−/− mice with pancreatic cancer compared to WT-PC (n=7, Student T-test). P=0.009. (6G) PC tumor growth is significantly more prominent in mice injected with AAV-GFP compared to AAV-HNF4□□(AAV-GFP n=4, AAV-HNF4α n=9, student T-Test) P=0.006. (6H) Mice with PC injected with AAV-HNF4α demonstrate a significant increase in survival compared to PC-AAV-GFP mice (Log-rank (Mantel-Cox) test) P=0.02. (6I) Mice with PC injected with AAV-HNF4□ maintain weight during CAC compared to mice with pancreatic cancer injected with AAV-GFP (AAV-GFP n=8, AAV-HNF4α n=9, 2-way ANOVA), P=0.05. (6J) NMR body composition analysis of mice with pancreatic cancer injected with AAV-HNF4α demonstrate less fat tissue loss (Left panel) and decreased free fluids accumulation (Right panel) in comparison to mice with PC injected with AAV-GFP mice (AAV-GFP n=5, AAV-HNF4α n=6, Student T-Test), P=0.007, 0.012, respectively.

FIGS. 7A-I: Routine liver tests predict weight loss in PDAC patients. (7A) Analysis of the Clalit Health Care database for liver biochemical tests demonstrates that non-metastatic PDAC patients with shorter survival time have abnormal liver parameters at the time of diagnosis compared to those with longer survival. (PDAC; n=2037-survival of <0.5y, n=659-survival of 0.5-1y, n=342, survival of 1-1.5y; **p<0.01, ***p<0.001). (7B) K.M. survival curves of PDAC patients from the Clalit database (Left) and from Sheba and Souraski medical centers (Right) demonstrate decreased survival in PDAC patients with high liver-function scores. P=0.0003 and P<0.0001, respectively. (7C-D) Analysis of data from the Sheba and Sourasky medical centers. (7C) Liver score does not correlate with pancreatic cancer disease stage. LA—locally advanced, MTX-metastatic disease. (7D) Correlation of weight loss during PDAC for high (red) and low (blue) liver scores of PDAC patients from Sheba and Sourasky Medical Centers, as binned by stage of disease at diagnosis. (Linear regression P=0.02). (7E-F) Non-metastatic BC patients with a shorter survival time have abnormal liver parameters at the time of diagnosis (7E) and one-year pre-diagnosis (7F) in comparison with those who have a longer survival (BC; n=4732-survival of <2y,n=4086-survival of 2-5y, n=3984, survival of 5-10y; *P<0.05, **P<0.01, ***P<0.001). (7G) Patients with an early higher weight loss (blue) show decreased survival compared to those who lost less weight (yellow), (log-rank P<0.0051), even when age and sex are controlled for (cox Hazard ratio=1.21, P<0.0084). (7H) K.M. survival curve of PC patients with high and low liver function scores from the initial (n=50) dataset used to train the model. The calculations are detailed in the method section. (7I) K.M. survival curve of PC patients with high and low liver-function scores Upper panels—Sourasky data. For resectable tumors, n=255, for local and distant spread n=362. Lower panels— Sheba data, for stage 1-2 n=82. For stages 3-4, n=169. (Left—patients with stage 1-2 resectable tumors at diagnosis, Right—patients with stage 3-4 with a local and distant spread at diagnosis), P values: Resectable=0.0255, survival LA=0.0007, survival stage 3-4=0.005.

FIG. 8: A graphical summary of the findings proposing a mechanism for the tumor-induced systemic metabolic changes in the livers of cancer patients and cancer-bearing mice that can lead to CAC.

FIGS. 9A-H: (9A) Photograph of fluorescence from livers and spleens of mice administered either the H4-LNP or the SM-LNP. Fluorescence is only detectable in livers that received the SM-LNP. (9B) Western blot of HNF4A expression in THLE-2 contacted with the SM-LNP. Actin is measured as a loading control. (9C-H) Line graphs of percent change in (9C, 9F) weight, (9D, 9G) fat and (9E, 9H) free fluids in mice that were first given pancreatic cancer cells (day 0) and then were administered (9C-E) a therapeutic dose of the HNF4A-LNP at day 21 or (9F-H) a prophylactic dose of the HNF4A-LNP at day 7.

### DETAILED DESCRIPTION OF THE INVENTION

The present invention, in some embodiments, provides methods of predicting clinical outcome in a subject suffering from cancer by measuring function of the urea cycle in the subject. Methods of detecting a non-liver cancer in a subject by measuring function of the urea cycle in the subject are also provided. Methods of treating or preventing cancer or treating or preventing cancer-associated cachexia are also provided. Synthetic lipid nanoparticles encapsulating an mRNA encoding for HNF4A and composition comprising same are also provided.

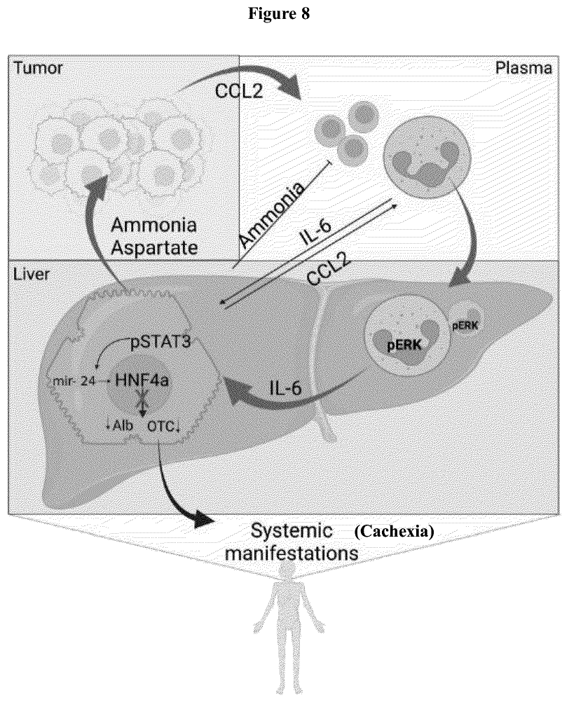

The invention is based, at least in part, on the surprising finding that while in the healthy liver exposure to foreign molecules results in regulated inflammation, following carcinogenesis and immune infiltration, the liver recruits innate immune cells by secreting an increasing amount of CCL2. Activated immune cells positive for pERK and elevated IL-6 levels lead to transcriptional changes in the expression of metabolic enzymes in the liver. Tumor induced IL-6 has been shown to impair the liver ketogenic response. Here we demonstrate a broad rewiring of liver metabolism via the IL-6-pSTAT3 immune-hepatic axis, which leads to the depletion of HNF4α, a master regulator of liver metabolism. Consequently, there are changes in systemic metabolism, increasing the availability of substrates which promote cancer growth and contributing to systemic manifestations such as weight loss and changes in body composition such as cachexia (FIG. 8).

Further, we propose a biochemical liver score that includes albumin and can predict survival and weight loss independent of the cancer stage. Therapeutically, our data indicates that giving clinically available drugs such as ERK inhibitors, STAT inhibitors, or anti-IL-6 blocking antibodies to the identified patients at risk at an early cancer stage can preserve liver metabolism and restrict cancer progression. Moreover, exogenous HNF4α can be used to maintain liver metabolism and limit systemic manifestations. This was achieved both with an HNF4α-AAV and a new liver targeting LNP comprising an optimized HNF4A mRNA (FIGS. 10C-H) which were able to not only shrink the cancer but prevent and treat cachexia.

By a first aspect, there is provided a method of detecting a cancer in a subject, the method comprising measuring function of the urea cycle in the subject, thereby detecting a cancer.

By a first aspect, there is provided a method of predicting clinical outcome in a subject, the method comprising measuring function of the urea cycle in the subject, thereby predicting clinical outcome.

In some embodiments, the method is an in vitro method. In some embodiments, the method is an ex vivo method. In some embodiments, the method is a diagnostic method. In some embodiments, the method is a prognostic method. In some embodiments, the method is a method of treatment. In some embodiments, the method is a method of detecting cachexia. In some embodiments, the method is a method of predicting the development of cachexia. In some embodiments, the method is a method of predicting the risk of developing cachexia. In some embodiments, the method is a method of predicting overall survival. In some embodiments, the method is a method of predicting mortality. In some embodiments, the method is a method of predicting years of survival. In some embodiments, the predicting is at least 1 month before the disease manifests. In some embodiments, the predicting is at least 3-months before the disease manifests. In some embodiments, the predicting is at least 6-months before the disease manifests. In some embodiments, the predicting is at least 1 year before the disease manifests. In some embodiments, the predicting is at least 2 years before the disease manifests. In some embodiments, the disease is cancer. In some embodiments, the disease is cachexia.

In some embodiments, the subject is a mammal. In some embodiments, the subject is a human. In some embodiments, the subject is in need of method of the invention. In some embodiments, the subject suffers from cancer. In some embodiments, the subject is at risk for developing cancer. In some embodiments, the subject suffers from cachexia. In some embodiments, the subject is at risk for developing cachexia. In some embodiments, the risk is determined or predicted by a method of the invention. In some embodiments, the cancer is detected by a method of the invention. In some embodiments, the cancer is predicted by a method of the invention. In some embodiments, the cachexia is detected by a method of the invention. In some embodiments, the cachexia is predicted by a method of the invention.

As used herein “cancer” or “pre-malignant lesion” refer to diseases associated with out of control cell proliferation. In some embodiments, the cancer is not liver cancer. In some embodiments, the cancer is a non-liver cancer. In some embodiments, the cancer does not comprise metastasis to the liver. In some embodiments, metastasis is detectable metastasis. In some embodiments, the cancer is a solid cancer. In some embodiments, the cancer is a hematological cancer. In some embodiments, the cancer is a tumor. In some embodiments, the cancer is selected from hepato-biliary cancer, cervical cancer, urogenital cancer, testicular cancer, prostate cancer, thyroid cancer, ovarian cancer, nervous system cancer, ocular cancer, lung cancer, soft tissue cancer, bone cancer, pancreatic cancer, bladder cancer, skin cancer, intestinal cancer, hepatic cancer, rectal cancer, colorectal cancer, esophageal cancer, gastric cancer, gastroesophageal cancer, breast cancer, renal cancer, skin cancer, head and neck cancer, leukemia and lymphoma. In some embodiments, the cancer is not hepatic cancer. In some embodiments, the cancer is not leukemia or lymphoma. In some embodiments, the cancer is breast cancer. In some embodiments, the cancer is pancreatic cancer.

In some embodiments, the cancer is at any stage. In some embodiments, the cancer is early-stage cancer. In some embodiments, the cancer is late-stage cancer. In some embodiments, the cancer is stage I cancer. In some embodiments, the cancer is stage II cancer. In some embodiments, the cancer is stage III cancer. In some embodiments, the cancer is stage IV cancer. In some embodiments, the cancer is a precancerous malignancy. In some embodiments, a malignancy is a malignant lesion. In some embodiments, the method is independent of cancer or pre-cancer stage. In some embodiments, the method is independent of cancer type other than being non-hepatic.

As used herein, the term “cachexia” refers to a wasting syndrome that leads to loss of skeletal muscle and fat. In some embodiments, cachexia further comprises increased free fluid. In some embodiments, cachexia is determined or diagnosed by measuring loss of fat, loss of muscle, increased free fluid or any combination thereof. In some embodiments, cachexia is cancer-associated cachexia. In some embodiments, cancer-associated cachexia is cancer-caused cachexia. Cachexia comprises three consecutive clinical stages: pre-cachexia, cachexia, and refractory cachexia. Pre-cachexia is defined as less than 5% of body weight loss. Cachexia is defined as the patient loses more than 5% of his body weight. The refractory cachexia phase is determined by a low WHO performance status score and a survival period of less than 3 months. In some embodiments, cachexia is pre-cachexia. In some embodiments, cachexia is second stage cachexia. In some embodiments, cachexia is not refractory cachexia. In some embodiments, cachexia is refractory cachexia.

In some embodiments, function of the urea cycle is measured in the subject. In some embodiments, function of the urea cycle is measured in a sample from the subject. In some embodiments, liver function is measured in the subject. In some embodiments, liver function is measured in a sample from the subject. In some embodiments, the method further comprises receiving a sample from the subject. In some embodiments, the sample is not a cancer sample. In some embodiments, the measurement is a measurement outside of the cancer. In some embodiments, the measurement is in the liver of the subject. In some embodiments, the sample is a liver biopsy. In some embodiments, the measurement is in blood of the subject. In some embodiments, a sample is a blood sample. In some embodiments, a blood sample is a whole blood sample. In some embodiments, a blood sample is a plasma sample. In some embodiments, a blood sample is a serum sample. In some embodiments, the sample is a urine sample. In some embodiments, the measuring function is based on the blood sample. In some embodiments, the detecting or determining is based on data from the sample. In some embodiments, the data from the sample is received.

In some embodiments, measuring urea cycle function comprises measuring expression of at least one urea cycle enzyme. In some embodiments, measuring liver function comprises measuring expression of at least one urea cycle enzyme. In some embodiments, measuring liver function comprises measuring expression of at least one liver enzyme. In some embodiments, the expression is protein expression. In some embodiments, the expression is mRNA expression. In some embodiments, the expression is in the liver of the subject. In some embodiments, the expression is in a liver sample from the subject. In some embodiments, the expression is in hepatocytes of the subject. In some embodiments, the expression is in the blood of the subject. Methods of measuring mRNA and proteins levels are well known, and any such method may be used. These methods include for example PCR, RT-PCR, qRT-PCR, RNA sequencing, western blotting, ELISA, immunostaining, protein arrays and many more. Commercial reagents and kits for performing this measuring are available.

In some embodiments, the at least one urea cycle enzyme is argininosuccinate synthetase 1 (ASS1). In some embodiments, the at least one urea cycle enzyme is ornithine transcarbamoylase (OTC). In some embodiments, the at least one urea cycle enzyme is argininosuccinate lyase (ASL). In some embodiments, the at least one urea cycle enzyme is carbamoyl phosphate synthetase-1 (CPS1). In some embodiments, the at least one urea cycle enzyme is ornithine translocase (ORNT1/SLC25A15). In some embodiments, the at least one urea cycle enzyme is selected from ASS1, OTC, ASL, CPS1 and ORNT1. In some embodiments, the at least one urea cycle enzyme is selected from ASS1, OTC, ASL and ORNT1. In some embodiments, OTC is measured. In some embodiments, OTC change is the earliest biomarker for urea cycle function. In some embodiments, mRNA levels of the at least one urea cycle enzyme are measured. In some embodiments, protein levels of the at least one urea cycle enzyme are measured. Exemplary primers for measuring mRNA expression of these enzymes and others are provided in Table 1. Exemplary antibodies for detecting these targets are as follows p97 (Thermo Fisher Scientific PA5-22257); ASS1 (Abcam ab124465); OTC (Abcam ab203859); Actin (Sigma-Aldrich A5441); TFAM (Cell Signaling #8076); pSTAT3 (Cell Signaling #9145); STAT3 (Cell Signaling #12640); HNF4α (Abcam ab181604); PCNA (Cell Signaling #13110); CAD (Cell Signaling #11933); pCAD (Cell Signaling #12662).

TABLE 1
Primers

| | | SEQ | | SEQ |
|---|---|---|---|---|
| | | ID | | ID |
| Target | Forward | NO: | Reverse | NO: |
| ASS1 | TGTTCGCCACTGTATCCAGAAGTC | 13 | CCGCTCCTCTTTGTCAGGGTCTA | 14 |
| OTC | ACACTGTTTGCCTAGAAAGCC | 15 | CCATGACAGCCATGATTGTCC | 16 |
| ASL | CGGACTCCTGATGACCCTCAAGT | 17 | TGTACTGTTCCACGCTGTGACTGT | 18 |
| CPS1 | ACATGGTGACCAAGATTCCTCG | 19 | TTCCTCAAAGGTGCGACCAAT | 20 |
| ORNT | AGATAGCGGCCAGTCAGAATA | 21 | CTCAGTTCATAGCCACCAAAGAA | 22 |
| ARG1 | TGTCCCTAATGACAGCTCCTT | 23 | GCATCCACCCAAATGACACAT | 24 |
| TFAM | AACACCCAGATGCAAAACTTTCA | 25 | GACTTGGAGTTAGCTGCTCTTT | 26 |
| HNF4A | CACGCGGAGGTCAAGCTAC | 27 | CCCAGAGATGGGAGAGGTGAT | 28 |
| CCL2 | AAAACACGGGACGAGAAACCC | 29 | ACGGGAACCTTTATTAACCCCT | 30 |
| ALB | TGCTTTTTCCAGGGGTGTGTT | 31 | TTACTTCCTGCACTAATTTGGCA | 32 |
| HPRT | TCTGGTAGATTGTCGCTTATCTTG | 33 | TAGATGCTGTTACTGATAGGAAATCGAG | 34 |
| D-LOOP1 | AATCTACCATCCTCCGTGAAACC | 35 | TCAGTTTAGCTACCCCCAAGTTTAA | 36 |
| ND4 | AACGGATCCACAGCCGTA | 37 | AGTCCTCGGGCCATGATT | 38 |
| CYT B | GCTTTCCACTTCATCTTACCATTTA | 39 | TGTTGGGTTGTTTGATCCTG | 40 |
| TERT | CTAGCTCATGTGTCAAGACCCTCTT | 41 | GCCAGCACGTTTCTCTCGTT | 42 |
| Glucagon | CAGGGCCATCTCAGAACC | 43 | GCTATTGGAAAGCCTCTTGC | 44 |
| miR-24 | UGGCUCAGUUCAGCAGGAACAG | 45 | | |

In some embodiments, the at least one liver enzyme is aspartate aminotransferase (AST). In some embodiments, the at least one liver enzyme is alanine aminotransferase (ALT). In some embodiments, the at least one liver enzyme is alkaline phosphatase (ALK-P/ALP). In some embodiments, the at least one liver enzyme is and lactate dehydrogenase (LDH). In some embodiments, the LDH is LDHA. In some embodiments, the LDH is LDHB. In some embodiments, the LDH is LDHA and LDHB. In some embodiments, the at least one liver enzyme is selected from AST, ALT, ALP and LDH. In some embodiments, the at least one liver enzyme is selected from ALP and LDH. In some embodiments, the at least one liver enzyme is selected from ALP, ALT and AST.

In some embodiments, measuring urea cycle function comprises measuring a urea cycle substrate. In some embodiments, the substrate is glutamate. In some embodiments, the substrate is aspartate. In some embodiments, measuring urea cycle function comprises measuring urea levels. In some embodiments, urea levels are levels in the urine. In some embodiments, urea levels are levels in the blood. In some embodiments, measuring urea cycle function comprises measuring urea to glutamine ratio. In some embodiments, measuring urea cycle function comprises measuring urea to glutamine ratio. In some embodiments, measuring urea cycle function comprises measuring a urea cycle metabolite. In some embodiments, a metabolite is a product. In some embodiments, the metabolite is fumarate. In some embodiments, the measuring is in the liver. In some embodiments, the measuring is in the blood.

In some embodiments, measuring urea cycle function comprises measuring ammonia levels. In some embodiments, ammonia levels are in the blood. In some embodiments, measuring urea cycle function comprises measuring albumin levels. In some embodiments, albumin levels are in the blood.

In some embodiments, measuring urea cycle function comprises measuring hepatocyte nuclear factor 4 alpha (HNF4A) expression. In some embodiments, expression is mRNA expression. In some embodiments, expression is protein expression. In some embodiments, expression is expression in the liver. In some embodiments, expression is expression in hepatocytes. Exemplary primers and antibodies for detecting HNF4A are provided hereinabove.

In some embodiments, measuring urea cycle function comprises measuring prothrombin. In some embodiments, measuring prothrombin is measuring prothrombin time. In some embodiments, prothrombin time is international normalized ratio (INR). In some embodiments, the INR is of the blood. In some embodiments, the measuring is in blood. In some embodiments, blood is a blood sample. The prothrombin time test is a well known clinical assay and its performance is a standard protocol known to one of skill in the art.

In some embodiments, decreased urea cycle function indicates a poor clinical outcome. In some embodiments, decreased urea cycle function indicates the presence of cancer. In some embodiments, decreased urea cycle function indicates the subject suffers from cancer. In some embodiments, decreased liver function indicates a poor clinical outcome. In some embodiments, decreased liver function indicates the presence of cancer. In some embodiments, decreased liver function indicates the subject suffers from cancer. In some embodiments, decreased is decreased below a predetermined threshold. In some embodiments, decreased is as compared to a control. In some embodiments, the predetermined threshold is the level/expression/value in a control. In some embodiments, a healthy control is a healthy control sample. In some embodiments, the sample or control is matched to the sample from the subject or the subject. That is, it will be understood by a skilled artisan that if the sample is a blood sample then the control will also be a blood sample, whereas if the measuring is in the liver the control will be measured in the liver. In some embodiments, the control is a healthy control. In some embodiments, a poor clinical outcome is a worse clinical outcome. In some embodiments, worse is as compared to a subject without the decreased urea cycle function. In some embodiments, worse is as compared to a subject without the decreased liver function. In some embodiments, worse is a as compared to a matched control without decreased function. That is if the subject suffers from cancer than the matched control suffers from the same cancer. Whereas if the subject has a pre-malignancy then the matched control also suffers from a pre-malignancy.

In some embodiments, decreased comprises at least a decrease of 5, 10, 15, 20, 25, 30, 35, 40, 45, 50, 55, 60, 65, 70, 75, 80, 85, 90, 92, 95, 97 99 or 100% decrease. Each possibility represents a separate embodiment of the invention. In some embodiments, decreased is at least a 25% decrease. In some embodiments, decreased is at least a 50% decrease. In some embodiments, the decrease is a detectable decrease. In some embodiments, the decrease is a significant decrease. In some embodiments, significant is statistically significant.

In some embodiments, decreased expression of the at least one urea cycle enzyme is indicative of decreased urea cycle function. In some embodiments, decreased expression of the at least one urea cycle enzyme is indicative of decreased liver function. In some embodiments, decreases expression of the at least one liver enzyme is indicative of decreased liver function. In some embodiments, decreased levels of the urea cycle substrate is indicative of decreased urea cycle function. In some embodiments, decreased levels of the urea cycle substrate is indicative of decreased liver function. In some embodiments, increased levels of a urea cycle metabolite is indicative of decreased urea cycle function. In some embodiments, increased levels of a urea cycle metabolite is indicative of decreased liver function. In some embodiments, a decrease in urea levels is indicative of decreased urea cycle function. In some embodiments, a decrease in urea levels is indicative of decreased liver function. In some embodiments, a decrease in urea to glutamine ratio is indicative of decreased urea cycle function. In some embodiments, a decrease in urea to glutamine ratio is indicative of decreased liver function. In some embodiments, a decrease in urea to glutamate ratio is indicative of decreased urea cycle function. In some embodiments, a decrease in urea to glutamate ratio is indicative of decreased liver function. In some embodiments, an increase in fumarate levels is indicative of decreased urea cycle function. In some embodiments, an increase in fumarate levels is indicative of decreased liver function. In some embodiments, an increase in ammonia levels is indicative of decreased urea cycle function. In some embodiments, an increase in ammonia levels is indicative of decreased liver function. In some embodiments, a decrease in albumin levels is indicative of decreased urea cycle function. In some embodiments, a decrease in albumin levels is indicative of decreased liver function. In some embodiments, a decrease in HNF4A expression is indicative of decreased urea cycle function. In some embodiments, a decrease in HNF4A expression is indicative of decreased liver function. In some embodiments, a decrease in INR is indicative of decreased urea cycle function. In some embodiments, a decrease in INR is indicative of decreased liver function.

In some embodiments, measuring liver function comprises producing a liver-function score. In some embodiments, the liver-function score is a sum of measures of liver function provided hereinabove. In some embodiments, the sum is a weighted sum. In some embodiments, the measures are normalized. In some embodiments, the sum is a sum of levels of at least two of AST ALT and ALP. In some embodiments, all three levels are summed. In some embodiments, the sum is a sum of levels of at least one of AST, ALT and ALP and the levels of albumin. In some embodiments, the sum is a sum of levels of at least two of AST, ALT and ALP and the levels of albumin. In some embodiments, the sum is a sum of levels of AST, ALT ALP and albumin. In some embodiments, the sum is a sum of levels of at least one of AST, ALT and ALP and the measured INR. In some embodiments, the sum is a sum of levels of Albumin and the measured INR. In some embodiments, the sum is a sum of levels of at least one of AST, ALT and ALP, the levels of albumen and the measured INR.

In some embodiments, the sum is a sum of levels of at least two of AST, ALT and ALP, the levels of albumen and the measured INR. In some embodiments, the sum is a sum of levels of AST, ALT ALP and albumin and the measured INR.

In some embodiments, at least one is at least two. In some embodiments, at least one is a plurality. In some embodiments, at least one is at least 1, 2, 3, 4, 5, 6, 7, 8, 9 or 10. Each possibility represents a separate embodiment of the invention. In some embodiments, at least one is all of.

In some embodiments, a score beyond a predetermined threshold indicates decreased urea cycle function. In some embodiments, a score beyond a predetermined threshold indicates decreased liver function. In some embodiments, urea cycle function is liver function. In some embodiments, liver function comprises urea cycle function. In some embodiments, a high score indicates decreased function and a low score indicates normal function. In some embodiments, beyond is above. In some embodiments, beyond is below. In some embodiments, the score is standardized from 0 to 1. In some embodiments, the score is on a scale from 0 to 1 or an equivalent. In some embodiments, the predetermined threshold is 0.6 on a scale from 0 to 1. In some embodiments, a score above 0.6 indicates decreases urea cycle function. In some embodiments, a score above 0.6 indicates decreases liver function.

In some embodiments, the clinical outcome is developing cachexia. In some embodiments, decreases urea cycle function is predictive of an increased risk of developing cachexia. In some embodiments, decreases urea cycle function is predictive that the subject will develop cachexia. In some embodiments, decreases liver function is predictive of an increased risk of developing cachexia. In some embodiments, decreases liver function is predictive that the subject will develop cachexia. In some embodiments, increased is increased as compared to a subject without a decrease function. In some embodiments, increased is increased as compared to a subject with a score that is not beyond the predetermined threshold. In some embodiments, increased is as compared to a subject without cancer, a precancerous lesion or an increased risk of developing cancer. In some embodiments, increased comprises an increase of at least 10, 15, 20, 25, 30, 35, 40, 45, 50, 55, 60, 65, 70, 75, 80, 85, 90, 95, 100, 125, 150, 175, 200, 250, 300, 350, 400, 450, or 500%. Each possibility represents a separate embodiment of the invention. In some embodiments, increase is by at least 20%. In some embodiments, increase is by at least 50%. In some embodiments, increase is by at least 100%.

In some embodiments, the clinical outcome is survival. In some embodiments, survival is overall survival. In some embodiments, decreases urea cycle function is predictive of reduced survival. In some embodiments, decreases liver function is predictive of reduced survival. In some embodiments, a score beyond the predetermined threshold is predictive of reduced survival. In some embodiments, reduced is as compared to a subject without decreased function. In some embodiments, reduced is as compared to a subject with a score within the predetermined threshold. In some embodiments, decreases survival is survival that is predicted to be less than a year. In some embodiments, decreases survival is survival that is predicted to be less than 2 year. In some embodiments, decreases survival is survival that is predicted to be from 2-5 years. In some embodiments, survival is survival at 1 year in the future. In some embodiments, survival is survival at 2 year in the future. In some embodiments, survival is survival at 5 year in the future. In some embodiments, survival is survival at 12 year in the future. In some embodiments, survival is survival at 13 year in the future. In some embodiments, survival is survival from diagnosis. In some embodiments, survival is survival from the performance of the method of the invention. In some embodiments, survival is survival when the cancer is resectable at diagnosis. In some embodiments, survival is survival when the cancer is metastasized at diagnosis. In some embodiments, survival is survival when the cancer is stage I-II at diagnosis. In some embodiments, survival is survival when the cancer is stage III-IV at diagnosis.

In some embodiments, the method further comprises administering to a subject determined to have cancer an anti-cancer treatment. Examples of anticancer treatments include, but are not limited to surgery, radiation therapy, chemotherapy, immunotherapy (e.g., immune checkpoint inhibitors), and targeted antibody therapy. In some embodiments, the method further comprises administering to a subject determined to have a worse clinical outcome an anti-cancer treatment. In some embodiments, the anticancer treatment is a therapeutic agent. In some embodiments, the anticancer therapy comprises administering a therapeutic agent.

In some embodiments, the anticancer therapy is anti-IL6 therapy. In some embodiments, the therapeutic agent is an anti-IL6 therapy. In some embodiments, anti-IL-6 therapy comprises administering an IL-6 blocking or neutralizing antibody. Examples of anti-IL6 antibodies include, but are not limited to Siltuximab, Olokizumab, Elsilimomab, Clazakizumab, Gerilimzumab, EBI-031 and Sirukumab. In some embodiments, anti-IL6 therapy comprises administering an IL-6 receptor (IL-6R) blocking or neutralizing antibody. Examples of anti-IL6R antibodies include, but are not limited to BCD-089, Tocilizumab, LusiNEX, Sarilumab and Vobarilizumab. As anti-IL-6 and IL-6R antibodies are so well known in the art, there is a sufficient number of members of the genus so as to represent the genus as a whole.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜ Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some embodiments, the anticancer therapy is ERK inhibition. In some embodiments, the therapeutic agent is an ERK inhibitor. In some embodiments, the anticancer therapy comprises administering an ERK inhibitor. Examples of ERK inhibitors include, but are not limited to CAS 1049738-54-6 (ERK Inhibitor), Trametinib, PD98059, SCH772984, tauroursodeoxycholate, patritumab, ulixertinib, reavoxertinib, astragaloside IV, tauroursodeoxycholate sodium, piperlongumine, temuterkib, lidocaine, BIX02189, FR180204, XMD8-92 and MK-8353 to name but a few. Other examples of ERK inhibitors can be found at medchemexpress.com/Targets/ERK, the contents of which are hereby incorporated by reference in its entirety.

In some embodiments, the anticancer therapy is STAT inhibition. In some embodiments, the therapeutic agent is a STAT inhibitor. In some embodiments, STAT is signal transducer and activator of transcription 3 (STAT3). In some embodiments, the anticancer therapy comprises administering a STAT inhibitor. Examples of STAT3 inhibitors include, but are not limited to Stattic, AG490, artesunate, niclosamide, cilengitide, STX-0119, STAT3-IN-15, homoharringtonine, C188-9, TPCA-1, napabucasin, cryptotanshinone, WP1066, NSC74859, SD-36, scutellarin, astaxanthin, and pimozide to name but a few. Other examples of STAT3 inhibitors can be found at medchemexpress.com/Targets/STAT/stat3, the contents of which are hereby incorporated by reference in its entirety.

In some embodiments, the inhibitor is a specific inhibitor. In some embodiments, a specific inhibitor does not substantially inhibit any protein other than the target (e.g., ERK or STAT). In some embodiments, substantially is significantly. In some embodiments, substantially is detectably. In some embodiments, the inhibitor or antibody is provided within a pharmaceutical composition. In some embodiments, the composition comprises a therapeutically effective carrier, excipient or adjuvant.

In some embodiments, the anticancer therapy comprises administering an agent that increases expression of HNF4A. In some embodiments, the therapeutic agent increases expression of HNF4A. In some embodiments, increases is increases within a liver of the subject. In some embodiments, within a liver is within a liver cell. In some embodiments, within a liver is within hepatocytes. In some embodiments, the agent is a lipid nanoparticle (LNP) of the invention.

In some embodiments, the agent comprises a nucleic acid molecule. In some embodiments, the agent is a nucleic acid molecule. In some embodiments, the nucleic acid molecule encodes for HNF4A. In some embodiments, the nucleic acid molecule comprises a coding region that encodes HNF4A. In some embodiments, the nucleic acid molecule comprises an open reading frame that encodes HNF4A.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, circulating nucleic acids, fragments of genomic DNA or RNA, degraded nucleic acids, ribozymes, viral RNA or DNA, nucleic acids of infectios origin, amplification products, modified nucleic acids, plasmidical or organellar nucleic acids and artificial nucleic acids such as oligonucleotides.

Hepatocyte nuclear factor 4 alpha (HNF4A) is also known as NR2A1, HNF4, TCF14, Nuclear Receptor Subfamily 2 group A member 1, Transcription Factor HNF-4 and Transcription Factor 14 among other names known in the art. In some embodiments, HNF4A is mammalian HNF4A. In some embodiments, HNF4A is rodent HNF4A. In some embodiments, the rodent is mouse. In some embodiments, HNF4A is human HNF4A. The mouse HNF4A gene can be found at Entrez #15378. The mouse HNF4A protein sequence can be found at Uniprot ID P49698. The RefSeq mRNA sequence for mouse HNF4A can be found in NM_008261, NM_001312906 and NM_001312907. The RefSeq protein sequence for mouse HNF4A can be found in NP_032287, NP_001299835 and NP_001299836. The human HNF4A gene can be found at Entrez #3172. The human HNF4A protein sequence can be found at Uniprot ID P41235. The RefSeq mRNA sequence for human HNF4A can be found in NM_000457, NM_001030003, NM_001030004, NM_001258355 and NM_001287182. The RefSeq protein sequence for human HNF4A can be found in NP_000448, NP_001025174, NP_001025175, NP_001245284 and NP_001274111.

In some embodiments, the mouse HNF4A cDNA coding sequence comprises atgcgactctctaaaacccttgccggcatggatatggccgactacagcgctgccctggacccagcctacaccaccctggagtttga aaatgtgcaggtgttgaccatgggcaatgacacgtccccatctgaaggtgccaacctcaattcatccaacagcctgggcgtcagtg ccctgtgcgccatctgtggcgaccgggccaccggcaaacactacggagcctcgagctgtgacggctgcaaggggttcttcagga ggagcgtgaggaagaaccacatgtactcctgcaggtttagccgacaatgtgtggtagacaaagataagaggaaccagtgtcgtta ctgcaggcttaagaagtgcttccgggctggcatgaagaaggaagctgtccaaaatgagcgggaccggatcagcacgcggaggt caagctacgaggacagcagcctgccctccatcaacgcgctcctgcaggcagaggttctgtcccagcagatcacctctcccatctct gggatcaatggcgacattcgggcaaagaagattgccaacatcacagacgtgtgtgagtctatgaaggagcagctgctggtcctgg tcgagtgggccaagtacatcccggccttctgcgaactccttctggatgaccaggtggcgctgctcagggcccacgccggtgagca tctgctgcttggagccaccaagaggtccatggtgtttaaggacgtgctgctcctaggcaatgactacatcgtccctcggcactgtcc agagctagcggagatgagccgtgtgtccatccgcatcctcgatgagctggtcctgcccttccaagagctgcagattgatgacaatg aatatgcctgcctcaaagccatcatcttctttgatccagatgccaaggggctgagtgacccgggcaagatcaagcggctgcggtca caggtgcaagtgagcctggaggattacatcaacgaccggcagtacgactctcggggccgctttggagagctgctgctgctgttgc ccacgctgcagagcatcacctggcagatgatcgaacagatccagttcatcaagctcttcggcatggccaagattgacaacctgctg caggagatgcttctcggagggtctgccagtgatgcaccccacacccaccaccccctgcaccctcacctgatgcaagaacacatg ggcaccaatgtcattgttgctaacacgatgccctctcacctcagcaatggacagatgtgtgagtggccccgacccagggggcagg cagccactcccgagactccacagccatcaccaccaagtggctcgggatctgaatcctacaagctcctgccaggagccatcacca ccatcgtcaagcctccctctgccattccccagccaacgatcaccaagcaagaagccatc (SEQ ID NO: 1). In some embodiments, the mouse HNF4A cDNA coding sequence consists of SEQ ID NO: 1. In some embodiments, the nucleic acid molecule comprises the mouse cDNA sequence. In some embodiments, the nucleic acid molecule comprises SEQ ID NO: 1 or a sequence with at least 85% homology thereto that encodes HNF4A. In some embodiments, homology is identity. In some embodiments, at least 85% is at least 90%, 92%, 95%, 97% or 99%. Each possibility represents a separate embodiment of the invention. In some embodiments, the nucleic acid molecule comprises SEQ ID NO: 1 operatively linked to at least one transcription regulatory element. In some embodiments, the RNA sequence of the coding sequence comprises SEQ ID NO: 2. In some embodiments, the RNA sequence of the coding sequence consists of SEQ ID NO: 2. In some embodiments, the nucleic acid molecule comprises the mouse mRNA coding sequence. In some embodiments, the nucleic acid molecule comprises SEQ ID NO: 2 or a sequence with at least 85% homology thereto that encodes HNF4A.

In some embodiments, the mouse HNF4A protein comprises MRLSKTLAGMDMADYSAALDPAYTTLEFENVQVLTMGNDTSPSEGANLNSSNSL GVSALCAICGDRATGKHYGASSCDGCKGFFRRSVRKNHMYSCRFSRQCVVDKDK RNQCRYCRLKKCFRAGMKKEAVQNERDRISTRRSSYEDSSLPSINALLQAEVLSQQ ITSPISGINGDIRAKKIANITDVCESMKEQLLVLVEWAKYIPAFCELLLDDQVALLR AHAGEHLLLGATKRSMVFKDVLLLGNDYIVPRHCPELAEMSRVSIRILDELVLPFQ ELQIDDNEYACLKAIIFFDPDAKGLSDPGKIKRLRSQVQVSLEDYINDRQYDSRGRF GELLLLLPTLQSITWQMIEQIQFIKLFGMAKIDNLLQEMLLGGSASDAPHTHHPLHP HLMQEHMGTNVIVANTMPSHLSNGQMCEWPRPRGQAATPETPQPSPPSGSGSESY KLLPGAITTIVKPPSAIPQPTITKQEAI (SEQ ID NO: 3). In some embodiments, the mouse HNF4A protein consists of SEQ ID NO: 3. In some embodiments, the coding region encodes SEQ ID NO: 3. In some embodiments, the HNF4A protein is a protein with at least 85% homology to SEQ ID NO: 3.

In some embodiments, the human HNF4A cDNA coding sequence comprises atgcgactctccaaaaccctcgtcgacatggacatggccgactacagtgctgcactggacccagcctacaccaccctggaatttga gaatgtgcaggtgttgacgatgggcaatgacacgtccccatcagaaggcaccaacctcaacgcgcccaacagcctgggtgtcag cgccctgtgtgccatctgcggggaccgggccacgggcaaacactacggtgcctcgagctgtgacggctgcaagggcttcttccg gaggagcgtgcggaagaaccacatgtactcctgcagatttagccggcagtgcgtggtggacaaagacaagaggaaccagtgcc gctactgcaggctcaagaaatgcttccgggctggcatgaagaaggaagccgtccagaatgagcgggaccggatcagcactcga aggtcaagctatgaggacagcagcctgccctccatcaatgcgctcctgcaggcggaggtcctgtcccgacagatcacctccccc gtctccgggatcaacggcgacattcgggcgaagaagattgccagcatcgcagatgtgtgtgagtccatgaaggagcagctgctg gttctcgttgagtgggccaagtacatcccagctttctgcgagctccccctggacgaccaggtggccctgctcagagcccatgctgg cgagcacctgctgctcggagccaccaagagatccatggtgttcaaggacgtgctgctcctaggcaatgactacattgtccctcggc actgcccggagctggcggagatgagccgggtgtccatacgcatccttgacgagctggtgctgcccttccaggagctgcagatcg atgacaatgagtatgcctacctcaaagccatcatcttctttgacccagatgccaaggggctgagcgatccagggaagatcaagcgg ctgcgttcccaggtgcaggtgagcttggaggactacatcaacgaccgccagtatgactcgcgtggccgctttggagagctgctgct gctgctgcccaccttgcagagcatcacctggcagatgatcgagcagatccagttcatcaagctcttcggcatggccaagattgaca acctgttgcaggagatgctgctgggagggtcccccagcgatgcaccccatgcccaccaccccctgcaccctcacctgatgcagg aacatatgggaaccaacgtcatcgttgccaacacaatgcccactcacctcagcaacggacagatgtccacccctgagaccccaca gccctcaccgccaggtggctcagggtctgagccctataagctcctgccgggagccgtcgccacaatcgtcaagcccctctctgcc atcccccagccgaccatcaccaagcaggaagttatc (SEQ ID NO: 4). In some embodiments, the human HNF4A cDNA coding sequence consists of SEQ ID NO: 4. In some embodiments, the nucleic acid molecule comprises the human cDNA sequence. In some embodiments, the nucleic acid molecule comprises SEQ ID NO: 4 or a sequence with at least 85% homology thereto that encodes HNF4A. In some embodiments, homology is identity. In some embodiments, at least 85% is at least 90%, 92%, 95%, 97% or 99%. Each possibility represents a separate embodiment of the invention. In some embodiments, the nucleic acid molecule comprises SEQ ID NO: 4 operatively linked to at least one transcription regulatory element. In some embodiments, the RNA sequence of the coding sequence comprises SEQ ID NO: 5. In some embodiments, the RNA sequence of the coding sequence consists of SEQ ID NO: 5. In some embodiments, the nucleic acid molecule comprises the human mRNA coding sequence. In some embodiments, the nucleic acid molecule comprises SEQ ID NO: 5 or a sequence with at least 85% homology thereto that encodes HNF4A.

In some embodiments, the human HNF4A protein comprises MRLSKTLVDMDMADYSAALDPAYTTLEFENVQVLTMGNDTSPSEGTNLNAPNSL GVSALCAICGDRATGKHYGASSCDGCKGFFRRSVRKNHMYSCRFSRQCVVDKDK RNQCRYCRLKKCFRAGMKKEAVQNERDRISTRRSSYEDSSLPSINALLQAEVLSRQ ITSPVSGINGDIRAKKIASIADVCESMKEQLLVLVEWAKYIPAFCELPLDDQVALLR AHAGEHLLLGATKRSMVFKDVLLLGNDYIVPRHCPELAEMSRVSIRILDELVLPFQ ELQIDDNEYAYLKAIIFFDPDAKGLSDPGKIKRLRSQVQVSLEDYINDRQYDSRGRF GELLLLLPTLQSITWQMIEQIQFIKLFGMAKIDNLLQEMLLGGSPSDAPHAHHPLHP HLMQEHMGTNVIVANTMPTHLSNGQMSTPETPQPSPPGGSGSEPYKLLPGAVATI VKPLSAIPQPTITKQEVI (SEQ ID NO: 6). In some embodiments, the human HNF4A protein consists of SEQ ID NO: 6. In some embodiments, the coding region encodes SEQ ID NO: 6. In some embodiments, the HNF4A protein is a protein with at least 85% homology to SEQ ID NO: 6.

In some embodiments, the nucleic acid molecule is a vector. In some embodiments, the vector is an expression vector. In some embodiments, the vector comprises at least one regulatory element operatively linked to a nucleic acid molecule of the invention. In some embodiments, the vector comprises at least one regulatory element operatively linked to an open reading frame encoding the antigen binding molecule of the invention. In some embodiments, the at least one regulatory element is a promoter.

The terms “operably linked” and “operatively linked” are used herein interchangeably and are intended to mean that the nucleotide sequence of interest is linked to the regulatory element or elements in a manner that allows for expression of the nucleotide sequence (e.g., in an in vitro transcription/translation system or in a host cell when the vector is introduced into the host cell).

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Expressing of a gene within a cell is well known to one skilled in the art. It can be carried out by, among many methods, transfection, viral infection, or direct alteration of the cell's genome. In some embodiments, the gene is in an expression vector such as plasmid or viral vector.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. In some embodiments, the vector is an AAV vector. The promoters may be active in mammalian cells. The promoter may be a viral promoter. In some embodiments, the promoter is a human promoter. In some embodiments, the promoter is a hepatocyte promoter.

In some embodiments, the HNF4A coding region is operably linked to a promoter. The term “operably linked” is intended to mean that the nucleotide sequence of interest is linked to the regulatory element or elements in a manner that allows for expression of the nucleotide sequence (e.g., in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, the nucleic acid molecule is an adeno-associated virus (AAV) vector. In some embodiments, the nucleic acid molecule is contained within an AAV. In some embodiments, the agent is an AAV comprising the nucleic acid molecule.

In some embodiments, the nucleic acid molecule is an mRNA. In some embodiments, the mRNA comprises a 5′ cap. In some embodiments, the mRNA comprises a 5′ untranslated region (UTR). In some embodiments, the 5′ UTR comprises a ribosome binding site. In some embodiments, the 5′ UTR comprises the 5′ end of the Kozak sequence (ACC). In some embodiments, the 5′ UTR comprises the 5′ end of the Kozak sequence (GCCACC). In some embodiments, the ACC is the 3′ end of the 5′ UTR. In some embodiments, the 5′ UTR comprises the T7 RNA promoter. In some embodiments, the T7 RNA promoter comprises the nucleotide sequence UAAUACGACUCACUAUA (SEQ ID NO: 46). In some embodiments, the T7 RNA promoter consists of SEQ ID NO: 46. In some embodiments, the 5′ terminus of the mRNA is the T7 RNA promoter. The T7 RNA promoter is commonly used in in-vitro transcription reactions. In some embodiments, the 5′ UTR comprises a human alpha globin mRNA 5′ UTR. In some embodiments, the human alpha globin 5′UTR comprises the nucleotide sequence GAAUAAACUAGUAUUCUUCUGGUCCCCACAGACUCAGAGAGAACCCGCCACC (SEQ ID NO: 47). In some embodiments, the 5′ UTR comprises the sequence UAAUACGACUCACUAUAAGGGAGACCCAAGCUGGCUAGCGUUUAAACUUAA GCUUGAAUAAACUAGUAUUCUUCUGGUCCCCACAGACUCAGAGAGAACCCG CCACCAAGGGAGACUCUUCUGGUCCCCACAGACUCAGAGAGAACCCACC (SEQ ID NO: 7). In some embodiments, the 5′ UTR consists of SEQ ID NO: 7. In some embodiments, the 5′ UTR comprises a sequence with at least 85% homology to SEQ ID NO: 7. In some embodiments, the sequence retains the ribosome binding site. In some embodiments, the sequence retains the 5′ end of the Kozak sequence at its 3′ terminus.

In some embodiments, the mRNA comprises a 3′ UTR. In some embodiments, the 3′ UTR enhances stability of the mRNA. In some embodiments, the 3′ UTR is derived from mitochondrial rRAN 3′ UTR sequence. In some embodiments, the 3′ UTR produces a thermodynamically stable secondary structure at the temperature used for LNP formation. In some embodiments, the temperature is about 37 degrees Celsius. In some embodiments, the temperature is between 50 and 70 degrees Celsius. In some embodiments, the 3′ UTR comprises the sequence GCUGGAGCCUCGGUGGCCAUGCUUCUUGCCCCUUGGGCCUCCCCCCAGCCCC UCCUCCCCUUCCUGCACCCGUACCCCCGUGGUCUUUGAAUAAAGUCUGAGUG GGCGGC (SEQ ID NO: 8). In some embodiments, the 3′ UTR consists of SEQ ID NO: 8. In some embodiments, the 3′ UTR comprises a sequence with at least 85% homology to SEQ ID NO: 8. In some embodiments, the nucleic acid molecule comprises a stop codon at the 3′ end of the coding region. In some embodiments, the region encoding HNF4A comprises a stop codon at its 3′ end. In some embodiments, the stop codon separates the coding region from the 3′ UTR. In some embodiments, the mRNA is poly-adenylated. In some embodiments, the mRNA comprises a poly-A tail. In some embodiments, the poly-A tail is 3′ to the 3′ UTR.

In some embodiments, the mRNA comprises a sequence encoding mouse HNF4A and comprises SEQ ID NO: 9. In some embodiments, the mRNA comprises a sequence encoding mouse HNF4A and consists of SEQ ID NO: 9. SEQ ID NO: 9 provides the DNA counterpart of the mRNA sequence. In some embodiments, the mRNA comprises a sequence encoding mouse HNF4A and comprises SEQ ID NO: 10. In some embodiments, the mRNA comprises a sequence encoding mouse HNF4A and consists of SEQ ID NO: 10. In some embodiments, the mRNA comprises a sequence encoding human HNF4A and comprises SEQ ID NO: 11. In some embodiments, the mRNA comprises a sequence encoding human HNF4A and consists of SEQ ID NO: 11. SEQ ID NO: 11 provides the DNA counterpart of the mRNA sequence. In some embodiments, the mRNA comprises a sequence encoding human HNF4A and comprises SEQ ID NO: 12. In some embodiments, the mRNA comprises a sequence encoding human HNF4A and consists of SEQ ID NO: 12.

In some embodiments, the nucleic acid molecule comprises a chemically modified backbone. In some embodiments, the RNA comprises a chemically modified backbone. Chemical modification of the backbone is known to enhance half-life and stability. In some embodiments, the chemically modified backbone comprises: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and any combination thereof.

In some embodiments, the nucleic acid molecule is encapsulated in a nanoparticle. In some embodiments, the nanoparticle is a nanoparticle of the invention.

By another aspect, there is provided a nucleic acid molecule that encodes HNF4A.

By another aspect, there is provided a nanoparticle that targets to the liver in a subject.

In some embodiments, the nanoparticle targets to the liver when administered systemically to the subject. In some embodiments, systemically is intravenously. In some embodiments, targeting to the liver comprises targeting to hepatocytes. In some embodiments, the nanoparticle targets to a mammalian liver. In some embodiments, the nanoparticle targets to a mouse liver. In some embodiments, the nanoparticle targets to a human liver.

In some embodiments, the nanoparticle comprises the agent. In some embodiments, the nanoparticle comprises the nucleic acid molecule. In some embodiments, the nanoparticle comprises the mRNA. In some embodiments, comprises is encapsulates. In some embodiments, the nanoparticle comprises an aqueous core. In some embodiments, the agent/nucleic acid molecule/mRNA is in the aqueous core. In some embodiments, in is dissolved in.

As used herein, a “nanoparticle” refers to a nano-sized carrier that can transport a nucleic acid molecule. In some embodiments, a nanoparticle comprises an average diameter of at most 750, 700, 650, 600, 550, 500, 450, 400, 350, 300, 250, 200, 175, 150, 140, 135, 130, 125, 120, 115, 110, 105, 100, 90, 80, 75, 70, 60 or 50 nanometers (nm). Each possibility represents a separate embodiment of the invention. In some embodiments, the nanoparticle comprises a diameter of at most 250 nm. In some embodiments, the nanoparticle comprises a diameter of at most 140 nm. In some embodiments, the nanoparticle comprises a diameter of at most 125 nm. In some embodiments, the nanoparticle comprises a diameter of at most 100 nm. In some embodiments, the nanoparticle comprises a diameter of at most 50 nm. In some embodiments, the average diameter is at least 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 nm. Each possibility represents a separate embodiment of the invention. In some embodiments, the average diameter is at least 50 nm. In some embodiments, the average diameter is at least 90 nm. In some embodiments, the average diameter is at least 100 nm. In some embodiments, the average diameter is between 50-700, 50-650, 50-600, 50-550, 50-500, 50-450, 50-400, 50-350, 50-300, 50-250, 50-200, 50-150, 50-140, 50-130, 50-120, 50-110, 50-100, 90-700, 90-650, 90-600, 90-550, 90-500, 90-450, 90-400, 90-350, 90-300, 90-250, 90-200, 90-150, 90-140, 90-130, 90-120, 90-110, 90-100, 100-700, 100-650, 100-600, 100-550, 100-500, 100-450, 100-400, 100-350, 100-300, 100-250, 100-200, 100-150, 100-140, 100-130, 100-120, 100-110, 150-700, 150-650, 150-600, 150-550, 150-500, 150-450, 150-400, 150-350, 150-300, 150-250, 150-200, 200-700, 200-650, 200-600, 200-550, 200-500, 200-450, 200-400, 200-350, 200-300, 200-250, 250-700, 250-650, 250-600, 250-550, 250-500, 250-450, 250-400, 250-350, or 250-300 nm. Each possibility represents a separate embodiment of the invention. In some embodiments, the average diameter is between 150 and 500 nm. In some embodiments, the average diameter is between 50 and 500 nm.

In some embodiments, the nanoparticle is a lipid nanoparticle (LNP). As used herein, the phrase “lipid nanoparticle” refers to a transfer vehicle comprising one or more lipids (e.g., cationic lipids, non-cationic lipids, and PEG-modified lipids). In some embodiments, the LNP is a liposome. In some embodiments, the LNP is a micelle. In some embodiments, the nanoparticle is a synthetic nanoparticle. In some embodiments, the nanoparticle is a man-made nanoparticle. In some embodiments, the nanoparticle is not a naturally occurring nanoparticle. In some embodiments, the LNP is not an exosome. In some embodiments, the LNP is not a naturally secreted vesicle.

Preferably, the lipid nanoparticles are formulated to deliver one or more agents (i.e., the nucleic acid molecule) to the liver/hepatocytes. Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a nucleic acid to a target cell.

The invention contemplates the use of lipid nanoparticles as transfer vehicles comprising a cationic lipid to encapsulate and/or enhance the delivery of nucleic acid into the target cell. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available.

Suitable cationic lipids for use in the compositions and methods of the invention include those described in international patent publication WO 2010/053572, incorporated herein by reference. In certain embodiments, the compositions and methods of the invention employ a lipid nanoparticles comprising an ionizable cationic lipid. In some embodiments, the cationic lipid N-[1-(2,3-dioleyloxy)propyl]-N,N,N-trimethylammonium chloride or “DOTMA” is used. (U.S. Pat. No. 4,897,355). DOTMA can be formulated alone or can be combined with the neutral lipid, dioleoylphosphatidyl-ethanolamine or “DOPE” or other cationic or non-cationic lipids into a liposomal transfer vehicle or a lipid nanoparticle, and such liposomes can be used to enhance the delivery of nucleic acids into target cells. Other suitable cationic lipids include, for example, 5-carboxyspermylglycinedioctadecylamide or “DOGS,” 2,3-dioleyloxy-N-[2(spermine-carboxamido)ethyl]-N,N-dimethyl-1-pr-opanaminium or “DOSPA” (U.S. Pat. Nos. 5,171,678; 5,334,761), 1,2-Dioleoyl-3-Dimethylammonium-Propane or “DODAP”, 1,2-Dioleoyl-3-Trimethylammonium-Propane or “DOTAP”. Contemplated cationic lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”, 1,2-dilinolenyloxy-N,N-dimethyl-3-aminopropane or “DLenDMA”, N-dioleyl-N,N-dimethylammonium chloride or “DODAC”, N,N-distearyl-N,N-dimethylammonium bromide or “DDAB”, N-(1,2-dimyristyloxyprop-3-yl)-N,N-dimethyl-N-hydroxyethyl ammonium bromide or “DMRIE”, 3-dimethylamino-2-(cholest-5-en-3-beta-oxybutan-4-oxy)-1-(cis,cis-9,12-oc-tadecadienoxy)propane or “CLinDMA”, 2-[5′-(cholest-5-en-3-beta-oxy)-3′-oxapentoxy)-3-dimethy 1-1-(cis,cis-9′,1-2′-octadecadienoxy)propane or “CpLinDMA”, N,N-dimethyl-3,4-dioleyloxybenzylamine or “DMOBA”, 1,2-N,N′-dioleylcarbamyl-3-dimethylaminopropane or “DOcarbDAP”, 2,3-Dilinoleoyloxy-N,N-dimethylpropylamine or “DLinDAP”, 1,2-N,N′-Dilinoleylcarbamyl-3-dimethylaminopropane or “DLincarbDAP”, 1,2-Dilinoleoylcarbamyl-3-dimethylaminopropane or “DLinCDAP”, 2,2-dilinoleyl-4-dimethylaminomethyl-[1,3]-dioxolane or “DLin-K-DMA”, 2,2-dilinoleyl-4-dimethylaminoethyl-[1,3]-dioxolane or “DLin-K-XTC2-DMA”, and 2-(2,2-di((9Z,12Z)-octadeca-9,12-dien-1-yl)-1,3-dioxolan-4-yl)-N,N-di-methylethanamine (DLin-KC2-DMA)) (See, WO 2010/042877; Semple et al., Nature Biotech. 28:172-176 (2010)), or mixtures thereof. (Heyes, J., et al., J Controlled Release 107: 276-287 (2005); Morrissey, D V., et al., Nat. Biotechnol. 23(8): 1003-1007 (2005); PCT Publication WO2005/121348A1). In some embodiments, the LNP comprises DOPE.

The use of cholesterol-based cationic lipids is also contemplated by the present invention. Such cholesterol-based cationic lipids can be used, either alone or in combination with other cationic or non-cationic lipids. Suitable cholesterol-based cationic lipids include, for example, DC-Chol (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE. In some embodiments, the LNP comprises cholesterol.

The use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000](C8 PEG-2000 ceramide) is also contemplated by the present invention, either alone or preferably in combination with other lipids together which comprise the lipid nanoparticle. The addition of such components may prevent complex aggregation and may also provide a means for increasing circulation lifetime and increasing the delivery of the lipid-nucleic acid composition to the target cell, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivitized lipids of the present invention may comprise a molar ratio from about 0% to about 20%, about 0.5% to about 20%, about 1% to about 15%, about 4% to about 10%, or about 2% of the total lipid present in the liposomal transfer vehicle. In some embodiments, the LNP comprises a PEGylated lipid. In some embodiments, the PEGylated lipid is PEGylated myristoyl diglyceride (DMG-PEG).

The present invention also contemplates the use of non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. Such non-cationic lipids may be used alone, but are preferably used in combination with other excipients, for example, cationic lipids. When used in combination with a cationic lipid, the non-cationic lipid may comprise a molar ratio of 5% to about 90%, or preferably about 10% to about 70% of the total lipid present in the transfer vehicle.

In some embodiments, the non-cationic lipid is an ionizable lipid. In some embodiments, the ionizable lipid is a synthetic amino lipid. Examples of ionizable lipids include, but are not limited to ALC-0315, SM-102, Lipid 5, DLin-DMA, D-Lin-MC3-DMA, DLin-KC2-DMA, YSK05, AA3-DLin, SSPalmM, SSPamO-Phe, Lipid A9, L319, CL4H6, DODMA, CL1, BP Lipid 308, ATX-100, 80-016B, 93-017S, (3-O17O and NT1-O14B to name but a few. In some embodiments, the LNP comprises SM-102.

In some embodiments, the lipid nanoparticle is prepared by combining multiple lipid and/or polymer components. The selection of cationic lipids, non-cationic lipids and/or PEG-modified lipids which comprise the lipid nanoparticle, as well as the relative molar ratio of such lipids to each other, is based upon the characteristics of the selected lipid(s) and their ability to target nucleic acid molecules (i.e., mRNA) to the liver. Additional considerations include, for example, the saturation of the alkyl chain, as well as the size, charge, pH, pKa, fusogenicity and toxicity of the selected lipid(s).

The LNPs for use in the compositions of the invention can be prepared by various techniques which are presently known in the art. Multi-lamellar vesicles (MLV) may be prepared conventional techniques, for example, by depositing a selected lipid on the inside wall of a suitable container or vessel by dissolving the lipid in an appropriate solvent, and then evaporating the solvent to leave a thin film on the inside of the vessel or by spray drying. An aqueous phase may then added to the vessel with a vortexing motion which results in the formation of MLVs. Uni-lamellar vesicles (ULV) can then be formed by homogenization, sonication or extrusion of the multi-lamellar vesicles. In addition, unilamellar vesicles can be formed by detergent removal techniques. In some embodiments, the LNP are produced by ethanol injection.

In some embodiments, the LNP comprises SM-102 and cholesterol. In some embodiments, the LNP comprises SM-102 and a PEGylated lipid. In some embodiments, the PEGylated lipid is DMG-PEG. In some embodiments, the LNP comprises SM-102 and a cationic lipid. In some embodiments, the cationic lipid DOPE. In some embodiments, the LNP comprises SM-102, cholesterol and DOPE. In some embodiments, the LNP comprises SM-102, cholesterol and DMG-PEG. In some embodiments, the LNP comprises SM-102, DOPE and DMG-PEG. In some embodiments, the LNP comprises SM-102, cholesterol, DOPE and DMG-PEG. In some embodiments, PEG is PEG200. In some embodiments, PEG is low molecular weight PEG.

In some embodiments, the LNP comprises between 40-60 mol % SM-102. In some embodiments, the LNP comprises between 45-55 mol % SM-102. In some embodiments, the LNP comprises about 50 mol % SM-102. In some embodiments, the LNP comprises between 30-50 mol % cholesterol. In some embodiments, the LNP comprises between 35-45 mol % cholesterol. In some embodiments, the LNP comprises between 33.5-43.5 mol % cholesterol. In some embodiments, the LNP comprises about 40 mol % cholesterol. In some embodiments, the LNP comprises about 38.5 mol % cholesterol. In some embodiments, the LNP comprises between 5-15 mol % DOPE. In some embodiments, the LNP comprises between 7.5-12.5 mol % DOPE. In some embodiments, the LNP comprises about 10% DOPE. In some embodiments, the LNP comprise between 0.5-2.5 mol % DMG-PEG. In some embodiments, the LNP comprise between 1-2 mol % DMG-PEG. In some embodiments, the LNP comprises about 1.5 mol % DMG-PEG. In some embodiments, the LNP comprises about 50 mol % SM-102, 38.5 mol % cholesterol, 10 mol % DOPE, and 1.5 mol % DMG-PEG200.

As used herein and in the art, mol percent (“% mol) refers to a percent of a particular component or compound based on the total mols of the components or compounds constituting the nanoparticle. For example, if a nanoparticle contains three mols of compound A and one mol of compound B, then the compound A comprises 75 mol % of the mixture and the compound B comprises 25 mol %.

By another aspect, there is provided a composition comprising the nanoparticle of the invention.

In some embodiments, the composition is a therapeutic composition. In some embodiments, the composition is a pharmaceutical composition. In some embodiments, the composition further comprises a pharmaceutically acceptable carrier excipient or adjuvant.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the composition is formulated for systemic administration. In some embodiments, the composition is formulated for intravenous administration. In some embodiments, the composition is formulated for administration to the liver. In some embodiments, the composition is formulated for hepatic administration. In some embodiments, the composition is formulated for administration to a subject. In some embodiments, the composition is formulated for administration to a human.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, intrahepatic, intramuscular, or intraperitoneal.

The dosage administered will be dependent upon the age, health, and weight of the recipient, kind of concurrent treatment, if any, frequency of treatment, and the nature of the effect desired.

By another aspect, there is provided a method of treating cancer in a subject in need thereof, the method comprising administering to the subject an agent capable of increasing expression of HNF4A in the subject, thereby treating cancer in the subject.

By another aspect, there is provided a method of treating cachexia in a subject in need thereof, the method comprising administering the subject a composition comprising at least one agent selected from: an anti-IL6 blocking antibody, an ERK inhibitor, a STAT3 inhibitor and an agent capable of increasing expression of HNF4A in the subject, thereby treating cachexia in the subject.

By another aspect, there is provided a method of preventing cachexia in a subject in need thereof, the method comprising administering the subject a composition comprising at least one agent selected from: an anti-IL6 blocking antibody, an ERK inhibitor, a STAT3 inhibitor and an agent capable of increasing expression of HNF4A in the subject, thereby preventing cachexia in the subject.

In some embodiments, the agent increases expression of HNF4A in the liver of the subject. In some embodiments, the agent increases expression of HNF4A in hepatocytes of the subject. In some embodiments, expression is protein expression. In some embodiments, increasing expression comprises delivering HNF4A to the liver or hepatocytes. In some embodiments, the agent is a nanoparticle of the invention.

In some embodiments, the composition is a pharmaceutical composition. In some embodiments, the composition is a composition of the invention. In some embodiments, the method comprises administering a nanoparticle of the invention. In some embodiments, the method comprises administering an LNP of the invention. In some embodiments, the method comprises administering a composition of the invention.

By another aspect, there is provided a nanoparticle of the invention for use in treating cancer. By another aspect, there is provided a nanoparticle of the invention for use in the production of a medicament for treating cancer.

By another aspect, there is provided a nanoparticle of the invention for use in treating cachexia. By another aspect, there is provided a nanoparticle of the invention for use in the production of a medicament for treating cachexia.

In some embodiments, the cancer is non-hepatic cancer. In some embodiments, the cachexia is cancer-associated cachexia. In some embodiments, the subject suffers from cancer. In some embodiments, the cancer is early-stage cancer. In some embodiments, the subject suffers from a pre-cancerous lesion. In some embodiments, the subject is at risk of developing cancer. In some embodiments, the subject is at risk of developing cachexia. In some embodiments, the subject has been determined to have the cancer by a method of the invention. In some embodiments, the subject has been diagnosed with cancer by a method of the invention. In some embodiments, the subject is determined to have cachexia by a method of the invention. In some embodiments, the subject is determined to be at risk for developing cachexia by a method of the invention. In some embodiments, the method further comprises determining the presence of decreased urea cycle function in the subject. In some embodiments, the treating is performed in a subject confirmed to have decreased urea cycle function. In some embodiments, the method further comprises determining the presence of decreased liver function in the subject. In some embodiments, the treating is performed in a subject confirmed to have decreased liver function. In some embodiments, determining decreased function is by a method of the invention.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

In some embodiments, treating comprises reducing the size of the tumor. In some embodiments, treating comprises reducing growth of the tumor. In some embodiments, reducing is halting. In some embodiments, treating comprises reducing at least one symptom of cachexia. In some embodiments, treating comprises reducing muscle loss of cachexia. In some embodiments, treating comprises reducing fat loss of cachexia. In some embodiments, treating comprises reducing free fluid of cachexia. In some embodiments, treating comprises at least one of increasing fat, increasing muscle and decreasing free fluid. In some embodiments, treating comprises increasing survival. In some embodiments, treating comprises increasing the time until development of cachexia. In some embodiments, treating comprises increasing survival beyond 1 year. In some embodiments, treating comprises increasing survival beyond 2 years. In some embodiments, treating comprises increasing survival beyond 5 years. In some embodiments, treating comprises increasing survival beyond 10 years.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

In-vivo animal studies: Animal experiments were approved by the Weizmann Institute Animal Care and Use Committee Following the U.S. National Institute of Health, European Commission, and the Israeli guidelines, To generate syngeneic mouse cancer models, 8-12 weeks old C57BL/6, or BALB/c male and female mice were purchased from Envigo and randomly assigned to experimental groups. For BC model, 8 weeks old BALB/c female mice were injected with 1×10⁶ 4T1 BC cells (in PBS) in the mammary fat pad. For PC model, 12 weeks old C57BL/6 male mice and CCR2-RFP knockout mice were injected with 0.3-0.4×10⁶ KPC PC cells (in DMEM 50% matrigel) in the pancreas tail. After sacrifice, livers, spleens, bone marrow and lungs were removed from the mice and blood was collected for further analysis by quantitative PCR, western blot and immunohistochemistry.

Cell lines: 4T1-luciferase cells derived from mouse breast cancer cells were kindly provided by Professor Yossi Yarden, Department of Biological Regulation, Weizmann Institute of Science. KrasG12D/Trp53R172H/Pdx-1-Cre (KPC)-luciferase cells derived from mouse solid PDAC were kindly provided by Professor Avigdor Scherz, Department of Plant and Environmental Sciences, Weizinann Institute of Science. All cells were tested routinely for Mycoplasma using Mycoplasma EZ-PCR test kit (#20-700-20, Biological Industries, Kibbutz Beit Ha'emek).

Western blotting: Tissues were grinded and lysed in RIPA (Sigma-Aldrich) and, 1% protease inhibitor cocktail (Calbiochem), 1% phosphatase inhibitor cocktail (P5726, Sigma-Aldrich). Following centrifugation, the supernatant was collected, and protein content was evaluated by the Bradford assay or BCA Protein Assay Kit (ThermoFisher Scientific, cat #23225). 20-50 μg from each sample under reducing conditions were loaded into each lane and separated by electrophoresis on a 10% SDS polyacrylamide gel. Following electrophoresis, proteins were transferred to Cellulose Nitrate membranes (Tamar, Jerusalem, Israel). Nonspecific binding was blocked by incubation with TBST (10 mM Tris-HCl (pH 8.0), 150 mM NaCl, 0.1% Tween 20) containing 5% skim milk for 1 h at room temperature. Membranes were subsequently incubated with antibodies (WB Antibodies list).

Antibody was detected using peroxidase-conjugated AffiniPure goat anti-rabbit IgG or goat anti-mouse IgG (Jackson ImmunoResearch, West Grove, PA) and enhanced chemiluminescence western blotting detection reagents (EZ-Gel, Biological Industries), Gels were quantified by Gel Doc XR+ (BioRad) and analyzed by ImageLab 5.1 software (BioRad). The relative intensity of each band was calculated by dividing the specific band intensity with the value obtained from the loading control.

Liver Perfusions and hepatocytes dissociation: Livers of anaesthetized mice were perfused, with specific adjustments. The vena cava was inserted with 27 G syringe, fixed on the perfusion line. 10 ml of pre-warmed to 42° C. PPML buffer followed by 25 ml of pre-warmed to 42′C PM buffer with Liberase (Roche, cat #05401127001) were perfused through the vena cava. Immediately at the beginning of the perfusion, the portal vein was cut. Following perfusion of 25 ml of PM buffer and Liberase solution, livers were harvested into a Petri dish with 10 ml of pre-warmed PM buffer and chopped by forceps. Dissociated liver cells were collected and filtered through a slanted 100 um cell strainer. Cells were spin down at 30 g for 3 min at 4° C. to get hepatocytes enriched pellet. Pellet was resuspended in 25 ul cold PM buffer. To enrich live hepatocytes, 21.6 ml cold Percoll (G.E. Healthcare #17-0891-01) mixed with +2.4 ml DPBSX10+CaCl2)+MgCl2 was added to the cells. Cells were centrifuged at 600 rpm for 10 minutes at 4° C. Supernatant containing the dead cells was aspirated, and cells were resuspended in 25 ml of cold Williams E+Glutamx-TM-1 (Gibco, cat #32551, 1% penstrep, 1% FBS, 1% L-Glutamine). Cells were centrifuged at 600 rpm for 5 minutes at 4° C. Supernatant was aspirated and cells were resuspended in 3 ml cold Williams E+Glutamx-TM-1.

Primary hepatocytes culture: Following perfusion, 1×10⁶ isolated hepatocytes were seeded in 3 ml Williams E+Glutamx-TM-1 in 6 well plate. Four hours after, the medium was aspirated, and the cells were washed with PBS and incubated with 2 ml Williams E+Glutamx-TM-1 overnight. Cells were treated with 10 μM of HJC0152 STAT3 inhibitor (Selleckchem #58561), for 30 minutes cells and then treated with 10 ng/ml recombinant mouse IL-6 (R&D #406-ML-005) for 48 h.

Histopathological and immunohistochemical staining analyses: Following 4, 14 and 21 days of 4T1 BC cells injection, and 7, 14 and 21 days of KPC PC cells injection, PFA fixed liver and lung tissues were embedded in paraffin blocks. The blocks were sectioned into 4 μm, and tissue sections were backed at 37° C. overnight. H&E staining was performed according to a standard protocol including the following steps: de-paraffinization, rehydration, staining with hematoxylin and eosin, followed by dehydration. The slides were cleaned with xylene and mounted.

Immunofluorescence: Following 21 days of 4T1 BC cells injection livers were collected, fixed in 4% paraformaldehyde, embedded in paraffin blocks and 4 m sections were made. Slides were de-paraffinized and antigen retrieval was done using Citric Acid PH=6. Blocking for unspecific binding was done with 20% Normal Horse Serum (NHS), 0.1% Triton in PBS. Rat anti CD45 (Bio Rad #MCA1031G) and mouse anti pERK 1:100 (Sigma #M8159) were diluted in 2% NHS and 0.1% Triton and were was incubated overnight. Slides were then incubated with Biotinylated donkey anti rat 1:100 (Jackson Immunoresearch #712-065-153) and HRP conjugated goat anti mouse 1:100 (Perkin Elmer #NEF822001EA) diluted 2% NHS for 1.5 hr. Slides were then incubated with 1:500 OPAL 690 (Akoya Biosciences #FP1497001KT) and Sterptavidin Cy3 (016-160-084—Jackson immunoresearch). Slides were imaged with Leica Mi8 microscope equipped with a motorized stage and a Leica DFC365 FX camera. Single ×20 magnification images were tiled to receive a full scan of the tumor section. The quantification in the liver sections stained with pERK was done by ImageJ.

RNA processing and quantitative PCR: RNA was extracted from liver tissue by using QIAzol Lysis Reagent (according to QIAzol® Handbook) or by Direct-zol™ MiniPrep Plus Kit (Zymo Research ZR-R2070). For hepatocytes RNA sequencing, RNA was extracted from dissociated hepatocytes. Following liver perfusion as previously described, hepatocytes were resuspended in QIAzol and frozen in −80° C. RNA was extracted by using QIAzol Lysis Reagent. RNA from cultured primary hepatocytes was extracted by using RNeasy Mini Kit (QIAGENe #74104). To evaluate mtDNA copy number, total DNA was isolated using DNA purification kit (DNeasy Blood & Tissue Kits Qiagen #69504). cDNA was synthesized from 1 g RNA by using qScript cDNA Synthesis Kit (Quanta #95749). Detection on cDNAs was performed using Syber Green Fast mix Perfect CT (Quantabio #95073) with the required primers (qPCR primer list—Table 1)

Perfusion for CyTOF and FACS assays: For blood collection, once anesthetized, mice were either injected with 10 μl of heparin on the left ventricle and ˜700 μl blood was withdrawn by cardiac puncture on the right ventricle using a 27 G needle in a 1 ml syringe coated with heparin or via retro-orbital bleeding procedure by using heparinized micro hematocrit capillary tubes, for immune cell isolation. Mice were intracardially perfused with ice-cold PBS. These assays were performed 14 days following the injection of 4T1 BC cells and control PBS, and 21 days following the injection of KPC PC cells and control PBS.

Blood immune cell isolation: Following perfusion, blood was withdraw and transferred into 15 ml tubes. 5 ml of red blood cell lysis buffer (ThermoFisher, ACK Lysing Buffer, A104920) was added and incubated at R.T. for 5-10 min. Blood was then centrifuged at 300 g for 5 minutes at 4° C. The supernatant was aspirated, and the pellet was resuspended in residual volume. Cells were washed with 5 ml ice-cold FACS buffer (Ca/Mg2 free PBS+2 mM EDTA+0.5% BSA or 5% FCS) and centrifuged at 300 g for 5 minutes at 4° C. The supernatant was aspirated, and the pellet was resuspended in residual volume.

Liver immune cell isolation: Following perfusion, livers were extracted and transferred into Petri dish. Livers were then minced into ˜1 mm pieces. 3 ml of DMEM-F12 (ThermoFisher, 31330038) was added to 15 ml tubes on ice. 3 ml of collagenase (Worthington, LS004188) cocktail (1 mg/ml Collagenase IV+0.2 mg/ml DNAse I+20% FBS in DMEM/gF12) was added and the tissue homogenates incubated at 37 C for 60 minutes shaking at 250 rpm, with brief vortex every 15 minutes. Cell suspension was filtered with 40 um strainer into a 50 ml tube and washed with 20 ml of ice-cold FACS buffer. Cells were then centrifuged at 600 g for 5 minutes at 4° C. The supernatant was aspirated and the pellet was resuspended in 5 ml red blood cell lysis buffer. At the end of the incubation 15 ml of ice cold FACS buffer was added, and samples were centrifuged at 600 g for 5 minutes at 4° C. Leukocyte enrichment based on Percoll gradient was performed. Isotonic Percoll (9 parts of Percoll, 1 part of sterile 10×PBS), 80% Percoll (8 parts of isotonic Percoll, 2 parts of 1×PBS), and 40% Percoll (5 parts of 80% Percoll, 5 parts of DMEM-F12) solutions were prepared. Pellet was resuspended in 8 ml of 40% Percoll and carefully transferred to 15 ml containing 5 ml of 80% Percoll. Cells were centrifuged at 1500 g for 30 minutes at 4° C. (acceleration 5/brake 0). The middle layer containing immune cells was collected and transferred into a new 15 ml tube containing 5 ml of ice-cold PBS buffer. Volumes were even out to 10 ml with ice-cold PBS and cells were centrifuged at 600 g for 5 minutes at 4° C. Supernatant was aspirated, and the pellet was resuspended in residual volume.

Spleen immune cell isolation: Following perfusion, the spleen was squashed over 70 um strainer and filtered with 10 ml FACS buffer. Cells were centrifuged at 400 g for 5 minutes at 4° C. The supernatant was aspirated, and cells were resuspended in 1 ml red blood cell lysis buffer. After 5 minutes incubation at R.T., 10 ml of ice-cold PBS were added and cells were centrifuged at 300 g for 5 minutes at 4° C. Supernatant was aspirated and the pellet was resuspended in residual volume.

Bone Marrow immune cells isolation: Following perfusion, the femur was dislocated and transferred into Petri dish containing ice-cold PBS. Condyles, patella, and epiphysis were removed to expose the metaphysis. The bone marrow (B.M.) was flushed with 2 ml of Medium over a 70 um strainer. B.M. was smashed with a syringe plunger and the filter was washed with 10 ml of RPMI+10% FBS+2 mM EDTA. Cells were centrifuged at 400 g for 5 minutes at 4° C. The supernatant was aspirated and the pellet was resuspended in 1 ml red blood cell lysis buffer for 5 minutes in R.T. 10 ml of RPMI+10% FBS+2 mM EDTA was added and centrifuged 400 g 5 min at R.T.

Flow cytometry: Immune cells were washed with ice-cold PBS and stained with LIVE/DEAD™ Fixable Aqua Dead (Thermo Fisher) according to the manufacturer's instructions. After Fc blocking (Biolegend, BLG-101320), cells were stained for surface antigen. Flow cytometry data were acquired on CytoFLEX (Beckman Coulter) and analyzed using FlowJo software. In each experiment, relevant negative, single-stained, and fluorescence-minus-one controls were used to identify the populations of interest.

Mass cytometry: 3×10⁶ cells per each liver sample, and all blood-derived immune cells were stained for mass cytometry analysis. Cisplatin viability stain was used prior to barcoding of samples with palladium metal isotopes. Briefly, individual samples were incubated with Human TruStain FcX™ (BioLegend), followed by staining with a panel of antibodies (CyTOF antibody list—Supplementary Table S6), for 30 min at room temperature (R.T), washed with 5 ml of Maxpar® Cell Staining Buffer, fixed with Fix I Buffer and permeabilized with Barcode Perm Buffer. Samples were then incubated with their respective barcodes for 45 min at R.T., after which they were washed with Maxpar® Barcode Penn Buffer and combined into a composite sample. After washing, the mixed sample was incubated with formaldehyde 4% overnight at 4° C. Before acquisition in a Helios™ II CyTOF® system, samples were stained with Iridium to detect cells and washed with cell staining buffer and mass cytometry grade water. Multidimensional datasets were analyzed using Cytobank cloud-based platform, FlowJo software (Tree Star, Inc) and R (R Core Team, 2017).

Algorithm-based high-dimensional analysis: Mass cytometry data were normalized and debarcoded with the Fluidigm CyTOF software version 6.7. Individual samples were manually gated using Cytobank to exclude normalization beads, cell debris, and dead cells. Only CD45+ cells were used for downstream analyses. All analyses on CyTOF data were performed after arcsinh (with cofactor=5) transformation of marker expression. Clustering, data visualization and dimension reduction (UMAP), were performed using the CyTOF workflow package. All plots were drawn using ggplot2 or GraphPad Prism (version 8.0.1).

L-Glutamine-15N2 infusion: Isotope infusion experiments were performed x weeks following orthotropic tumors inoculation. The mice were fasted for 4 hours followed by 5 hours of infusion with L-Glutamine-15N2 (Sigma). Infusion solutions containing 1.725 grams of L-Glutamine-15N2 per kg of body weight were prepared in saline. The mice were anesthetized on a heating pad, and a catheter, connected to the infusion solution was inserted in the lateral tail vein. Each mouse was initially infused with a bolus of 150 μl/min for 1 min, followed by continuous infusion of 2.5 μl/min for 5 hours. Mice were kept awake throughout the infusion in individual infusion cages. At the end of the infusion, mice were anesthetized and blood was collected into heparin tubes. Organs were harvested and snap-frozen in liquid nitrogen.

Gas-chromatography mass-spectrometry (GC-MS): Plasma was collected from blood samples through centrifugation at 1000 rcf for 15 minutes at 4° C. 20 μl of plasma was re-suspended in ice-cold MeOH/H2O mixture, 8:1 with Ribitol, incubated on ice for 20 minutes and centrifuged for 10 minutes at 15,000 rpm. The supernatants were vacuum dried over-night and the dried samples were incubated with 20 μl of methoxyamine hydrochloride solution (20 mg ml-1 in pyridine) at 37° C. for 90 min following by incubation of 40 ul of N-tert-Butyldimethylsilyl-N-methyltrifluoroacetamide with 1% tert-Butyldimethylchlorosilane (Sigma) for 60 minutes at 60° C.

Tissues samples of about 5-25 mg were vacuum dried, and powdered using 2 stainless still 3.2 mm balls on the cryomill (Retscht). The powdered tissues were re-suspended with methanol and ribitol as internal standard and sonicated for 20 minutes. Polar metabolites were extracted following addition of 1 volume of water and 0.5 volume of chloroform. The mixture was vortexed and centrifuge at 15,000 RPM for 15 minutes at 4° C. The samples' supernatants were dried overnight before dervitization with 40 ul methoxyamine hydrochloride solution (20 mg ml-1 in pyridine) at 37° C. for 90 min while shaking followed by incubation with 70 ml N,O-bis(trimethylsilyl) trifluoroacetamide (Sigma) at 37° C. for an additional 30 min.

1 ul of sample was injected either in splitless or in 1:25 split mode, using an inlet temperature of 270° C., the GC oven was held at 100° C. for 3 min and then ramped to 300° C. with a gradient of 3° C. min−1 followed by a 5 min after run at 315° C. The MS system was operated under electron impact ionization at 70 eV and a mass range of 100-650 amu was scanned. The resulting chromatograms were analyzed in MassHunter software (Agilent Technologies). Isotopologue distribution of the metabolites was corrected for naturally occurring isotopes using IsoCor software.

Amino acid analyzer: Frozen liver samples were lyophilized and grounded to powder, extracted with 50% methanol homogenized in bullet blunder following 10 minutes of sonication in ice cold water. The samples were centrifuged at 15,000×g for 15 min and lyophilized again as described in the GCMS section. The samples were resuspended with lithium loading buffer (Biochrom) and proteins were precipitated by addition of (v/v) cold 5% 5-Sulphosalicylic acid (SSA) solution supplemented with 500 μmol/L of Norleucine as internal standard. The mixtures were incubated for 30 min at 4° C. following centrifugation at 15,000×g for 15 min. The supernatants were filtered through a 0.22-μm—size filter and subsequently injected onto Biochrom 30 series amino acid analyzer (Biochrom Ltd., Cambridge Science Park, England) with a Lithium Accelerated cation-exchange column (200×4.6). A mixture of amino acids at known concentrations (Calibration standards, Biochrom) was supplemented with glutamine and used as standard. Amino acids were post-column derivatized with ninhydrin reagent and detected by absorbance at 440 nm (proline and hydroxyproline) or 570 (all the other amino acids) nm.

Extraction of polar metabolites from urine and plasma: To extract polar metabolites from urine (20˜100 uL) samples, 1 mL methanol (with labeled amino acids as internal standard) were added, respectively, into biological sample-containing Eppendorf tube. Then, the resulting mixture was vortexed and sonicated for 15 min, vortexed again, and centrifuged at 14000 rpm for 10 min. The liquid phase was transferred into new tube and lyophilized. Then the pellets were dissolved using 150 uL DDW-methanol (1:1), centrifuged twice to remove possible precipitants, and was injected into LC-MS system.

LC-MS polar metabolites analysis: Briefly, analysis was performed using Acquity I class UPLC System combined with mass spectrometer (Thermo Exactive Plus Orbitrap) which was operated in a negative ionization mode. The L.C. separation was done using the SeQuant Zic-pHilic (150 mm×2.1 mm) with the SeQuant guard column (20 mm×2.1 mm) (Merck). TheMobile phase A: acetonitrile and Mobile phase B: 20 mM ammonium carbonate plus 0.1% ammonia hydroxide in water. The flow rate was kept at 200 μl min−1 and gradient as follow: 0-2 min 75% of B, 17 min 12.5% of B, 17.1 min 25% of B, 19 min 25% of B, 19.1 min75% of B, 19 min 75% of B.

Polar metabolites data analysis: The data processing was done using TraceFinder Thermo Fisher software were detected compounds were identified by Retention time and fragments and verified using in-house mass spectra library. Urine metabolites were normalized by creatinine peak area.

Cytokines detection: Cytokine levels were measured by either ProcartaPlex Immunoassays (ThermoFisher ProcartaPlex™ Panel) or by IL-6 ELISA kit (ThermoFisher 88-7064-22) according to the manufacture instructions.

CCL2 and Ammonia levels: CCL2 and ammonia levels were measured by CCL2 ELISA kit (R&D Systems #MJE00B) and Ammonia Assay Kit (Abcam ab83360), respectively, according to manufacturer instructions.

Activation of T cells from mice spleens: 10 weeks old WT female Balb/c mice were sacrificed and spleen harvested into cold-PBS on ice. Spleens were homogenized by syringe plunger through 70 uM strainer and washed with PBS. Following centrifugation at 1200 rpm for 5 min pellets were treated with RBC lysis buffer according to the manufacturer's instruction. Cells were resuspended in 2×10⁶ cells/ml in splenocytes medium (complete RPMI medium supplemented with 50 μM β-mercaptoethanol, 10% Sodium Pyruvate and Non-essential amino acids) supplemented with 6,000 IU/mL IL-2 (Chiron, rhIL2) and seeded in 24 well plates pre-coated with CD3 (BLG #100302). Following 72 h, cells were collected, centrifuged at 1200 rpm for 5 min, washed with splenocytes medium×2, and analyzed using CytoFLEX (Beckman Coulter) FACS analyzer.

Cancer-cells' proliferation: 20×10⁴ 4T1 cells were seeded in 100 ul of complete RPMI medium. On the following day, cells were washed with PBS and the medium was replaced with DMEM glutamine-free medium (Biological Industries #01-057-1A), supplemented with ammonia (0.75 mM), aspartate (0.25 mM), fumarate (0.35 mM), or glutamine (0.25 mM). Proliferation assays (XTT cell proliferation kit Biological Industries #20-300-1000) was used according to manufacturers' instructions 24, 48, and 72 hr following metabolites supplementation.

Measurements of respiratory chain complexes activity: The enzymatic activities of respiratory chain complexes were measured at 37° C. by standard spectrophotometric methods. Briefly, Complex I was measured as rotenone sensitive NADH-CoQ reductase monitoring the oxidation of NADH at 340 nm in the presence of coenzyme Q1. Complex II was measured as succinate dehydrogenase (SDH) based on the succinate-mediated phenazine methosulfate reduction of dichloroindophenol at 600 nm. Complex II+III was measured as succinate cytochrome c reductase and after the reduction of oxidized cytochrome c at 550 nm. Complex IV (cytochrome c oxidase) was measured by following the oxidation of reduced cytochrome c at 550 nm. Citrate synthase (C.S.), a ubiquitous mitochondrial matrix enzyme, was measured in the presence of acetyl-coA and oxaloacetate by monitoring the liberation of CoASH coupled to 5′,5′-dithiobis (2-nitrobenzoic) acid at 412 nm. Protein concentration was determined by the Lowry method and calculated according to a bovine serum albumin (BSA) standard curve.

In-vivo Erk Inhibition: Following 24 h of 4T1 BC cells injection, mice were injected I.P. with 1 mg/kg ERK inhibitor Trametinib GSK1120212 (Selleckchem #S2673) in 4% DMSO corn oil or 4% DMSO corn oil only for 6 more times a week. Mice were sacrificed 8 or 14 days following tumor injection.

In vivo IL-6 inhibition: Following four days of KPC cells injection, mice were injected I.P. with 200 ug/mice of IL-6 Ab (InVivoMab anti-mouse IL-6 (Bio X Cell) #BE0046) or control IgG (InVivoMab rat IgG1 isotype control (anti-HRP) (Bio X Cell) #BE0088) every 2 days. Mice were sacrificed 21 days following tumor injection.

rAAV-HNF4α: Cells—Low passage HEK293T were maintained at 37° C. with 5% CO2 in Dulbecco's modified Eagle's medium supplemented with 10% fetal bovine serum. Production of rAAV—To produce rAAV8, a triple co-transfection procedure was used to introduce a rAAV vector plasmid (pAAV-CMV-mHNF4α or pAAV-CMV-GFP) together with pXR8, AAV8 helper plasmid carrying AAV rep and cap genes and pXX6-80, Ad helper plasmid, at a 1:1:1 molar ratio.

Briefly, HEK293T cells were transfected using poly-ethylenimine (PEI) (linear; molecular weight [M.W.], 25,000) (Poly-sciences, Inc., Warrington, PA), and medium was replaced at 18 h post-transfection. Cells were harvested at 72 h post-transfection, subjected to 3 rounds of freeze-thawing, and then digested with 100 U/ml Benzonase (EMD Millipore, Billerica, MA) at 37° C. for 1 h. Viral vectors were purified by iodixanol (Serumwerk Bernburg AG, Germany) gradient ultracentrifugation. followed by further concentration using Amicon ultra-15 100K (100,000-molecular-weight cutoff, Merck Millipore, Ireland) and washed with phosphate-buffered saline (PBS −/−). Final concentration of rAAV8 particles was 2.78E+10 vg per microliter (AAV-CMV-mHNF4α) and 2.35E+10 vg per microliter (pAAV-CMV-GFP). Mice were injected via tail vain with 5E11 vg 48 hr following inoculation with cancer cells.

RNA-seq: Total RNA was fragmented, followed by reverse transcription and second strand cDNA synthesis. The double strand cDNA was subjected to end repair, a base addition, adapter ligation and PCR amplification to create libraries. Libraries were evaluated by Qubit and TapeStation. Sequencing libraries were constructed with barcodes to allow multiplexing of 12 samples on a one lanes of Illumina HiSeq 2500 V4 instrument, resulting in ˜23 million single-end 60-bp reads per sample. Bioinformatics: Poly-A/T stretches and Illumina adapters were trimmed from the reads using cutadapt [doi:10.14806/ej.17.1.200]; resulting reads shorter than 30 bp were discarded. Reads were mapped to the M. musculus reference genome GRCm38 using STAR, supplied with gene annotations downloaded from Ensembl (with the option EndToEnd and outFilterMismatchNoverLmax was set to 0.04). Expression levels for each gene were quantified using htseq-count, using the gtf above. Differentially expressed genes were identified using DESeq2 with the betaPrior, cooksCutoff and independentFiltering parameters set to False. Raw P values were adjusted for multiple testing using the procedure of Benjamini and Hochberg. The pipeline was run using snakemake.

Principal component analysis was performed (using the R Stats package) on the DESeq2 variance stabilizing transformed values of the 1,000 most variable genes. The figure depicts the first versus the second principal component in a scatter plot. The first PC explains 73% and the second PC explains 9% of the variance of the data. Heatmap analysis was performed on total of 2829 genes that came up significant in any of the comparisons (4T1 breast bearing mice and CTRL WT mice on day 4 and 21) are shown. A gene was considered to be significant if its absolute fold change was above 1.5, FDR below 0.05 and the gene had account of at least 30 at least in one of the samples. The log 2 normalized counts, were standardized to have for each gene zero mean and unit standard deviation. Gap Statistic was used for estimating the number of Clusters. K-means clustering of the standardized values was performed. The expression profile is accompanied by a colored bar indicating the standardized log 2 normalized counts. For pathway enrichment analysis, we used the QIAGEN's Ingenuity® Pathway Analysis. Identifying genes under HNF4α regulation—Differentially expressed genes between 4T1 hepatocytes in day 21 and day 4 were calculated and normalized to control mice in both time points (|log FC|>=1.5 and FDR<0.05). 1914 genes were upregulated in day 21 time point compared to day 4, and 514 genes were downregulated at this time point. The list of downregulated genes was crossed with the list of target genes of HNF4α transcription factor from the Harmonizome tool (https://maayanlab.cloud/Harmonizome/gene_set/HNF4A/ENCODE+Transcription+Factor+Targets) built using ChIP-seq datasets from the ENCODE Transcription Factor Targets dataset. The final list contained 149 genes. The RNA-Seq data discussed in this publication have been deposited in NCBI's Gene Expression Omnibus and are accessible through GEO Series accession number GSE212113 (ncbi.nlm.nih.gov/geo/query/acc.cgi?acc=GSE212113).

Single cell RNA-seq using Chromium 10× genomics platform: For liver' NPCs enrichment, the liver of mice injected with PBS or 4T1 BC cells 4 and 21 days after injection, were perfused as previously described above. Following 3 min of centrifugation at 30 g supernatant was collected and centrifuged at 300 g for 5 min. Cells pellet was treated with red blood cell lysis buffer (ThermoFisher, ACK Lysing Buffer #A104920) according to the manufacturer's instruction. Single cell RNA-seq libraries were prepared using the chromium single cell RNA-seq platform (10× genomics). Cells were counted and diluted to a final concentration in PBS supplemented with 0.04% BSA. Cellular suspension was loaded onto Next GEM Chip G targeting liver non parenchymal cells and then ran on a Chromium Controller instrument to generate GEM emulsion (10× Genomics). Single-cell 3′ RNA-seq libraries were generated according to the manufacturer's protocol (10× Genomics Chromium Single Cell 3′ Reagent Kit User Guide v3 Chemistry). Final libraries were quantified using NEBNext Library Quant Kit for Illumina (NEB) and high sensitivity D1000 TapeStation (Agilent). Libraries were pooled according to targeted cell number, aiming for ˜50,000 reads per cell. Pooled libraries were sequenced on a NovaSeq 6000 instrument using an SP 100 cycles reagent kit (Illumina). The scRNA-Seq data discussed in this publication have been deposited in NCBI's Gene Expression Omnibus and are accessible through GEO Series accession number GSE223835 (ncbi.nlm.nih.gov/geo/query/acc.cgi?acc=GSE223835).

Single-cell RNA analysis: Metacell pipeline—The metacell pipeline36, was used to derive informative genes and to compute cell-to-cell similarity, to compute k-NN graph covers and derive distribution of RNA in cohesive groups of cells (or metacells) and to derive strongly separated clusters using bootstrap analysis and computation of graph covers on resampled data. We removed specific mitochondrial genes, immunoglobulin genes and genes linked with poorly supported transcriptional models (annotated with the prefix ‘Rp-’). Gene features were selected using the threshold value for the normalized var/mean (Tvm)=0.3 and a minimum total UMI count >50. We subsequently performed hierarchical clustering of the correlation matrix between metacells and grouped them into clusters representing cell types and states. We used K=100, 500 bootstrap iterations and otherwise standard parameters.

Neutrophil's subsets analysis—Neutrophil's subsets were identified according to the maturation score and chemotaxis score, as well as granules identification. Pathway analysis—G.O.—To evaluate pathways enriched in DEGs we used G.O. gene ontology 37, 38, 39 for genes differentially upregulated in macrophages on day 21 compared to day 0 and for HNF4α regulated genes out of differentially downregulated genes in hepatocytes between day 21 and day 4. Statistical analyses Differential gene expression analysis was performed on UMIs divided by the median UMI count using a Mann-Whitney Utest with FDR correction.

Human data from Clalit Healthcare: The analysis of the data from the Clalit Healthcare environment was under Helsinki approval no. 195-17COM2. Cohorts definitions for breast cancer—Female patients with diagnosis of breast cancer (all stages, both right and left breast, all quadrants, all histological subtypes) between the years 2002-2019 were included in breast cancer cohorts. Cohort 1: Breast cancer patients with survival of less than 2 years from diagnosis, N=4732.Cohort 2: Breast cancer patients with survival between 2 and 5 years from diagnosis, N=4086.Cohort 3: Breast cancer patients with survival between 5 and 10 years from diagnosis, N=3984.Cohort 4: Breast cancer patients with a CBC test within one year of diagnosis (−365 days before diagnosis to 365 days post diagnosis) indicating percentage of neutrophils higher than 80% or percentage of lymphocytes lower than 10%. N=10556.Cohort 5:Breast cancer patients with a CBC test within one year of diagnosis (−365 days before diagnosis to 365 days post diagnosis) indicating percentage of neutrophils lower than 80% and percentage of lymphocytes higher than 10%, N=35723. Cohorts definitions for pancreatic cancer—Male patients with diagnosis of pancreatic cancer (all stages, all locations in the pancreas, all histological subtypes) between the years 2002-2019 were included in pancreatic cancer cohorts. Cohort 1: Pancreatic cancer patients with survival of less than half a year from diagnosis, N=2037.Cohort 2: Pancreatic cancer patients with survival between half a year and 1 year from diagnosis, N=659.Cohort 3: Pancreatic cancer patients with survival between 1-1.5 years from diagnosis, N=342.Cohort 4: Pancreatic cancer patients with a CBC test within one year of diagnosis (−365 days before diagnosis to 365 days post diagnosis) indicating percentage of neutrophils higher than 80% or percentage of lymphocytes lower than 10%. N=4238.Cohort 5: Pancreatic cancer patients with a CBC test within one year of diagnosis (−365 days before diagnosis to 365 days post diagnosis) indicating percentage of neutrophils lower than 80% and percentage of lymphocytes higher than 10%, N=4218. Liver function lab data—For cohorts 1-3 of each cancer type, where available, results of lab tests for selected variables taken in 2 time points (One year before diagnosis, and at the time of diagnosis) were obtained for each patient between the ages of 60-70. The variables for liver function: AST (aspartate aminotransferase), ALT (alanine aminotransferase), Albumin, ALK-P (alkaline phosphatase), LDH (lactate dehydrogenase), PT-SEC. Survival analysis For cohorts—4-5 of each cancer type, pct of patients alive in the end of every year (0=time of diagnosis) was calculated and a survival curve was created as an X-Y plot of % live patients at each year.

Score for liver function in pancreatic patients' data: Sheba and Souraski medical centers Data on participants' demographics, past surgical procedures, blood test values and survival were extracted from patients' medical records under IRB-approval (4474 & 5073-18 & 0551-17-TLV). Written informed consent was obtained from all patients prior to study enrollment. The protocols were approved by the Institutional Review Board at the Sheba and Sourasky Medical Centers, and the studies were conducted in accordance with the Good Clinical Practice guidelines and the Declaration of Helsinki. Pancreatic cancer patients with either liver metastases or ascites were excluded to perform the analysis on non-liver cancer data. Souraski: N=732 (N for resectable=255, N for LA and MTX=362).Sheba: N=252 (N for stages 1+2=82, N for stages 3+4=170). For each sample, we then determine its liver function score, which is a weighted sum of the normalized expression of 5 liver enzymes and function-based molecules, the normalization into the average and std in pancreatic cancer patients (as studied from this cohort), and the weights defined based on correlation of each value with the survival of a random small cohort (50 patients), i.e.2*|ALT(IU/I)−25.5|19.5+2*|AST(IU/I)−25|15+5*|INR−1.02510.175+|ALKP(IU/I)−79.5|35.5+7*|4.46−Albumin(g/dL)|0.8617 where the names of genes denote their lab result value. A cutoff to separate high from low score was set at Cutoff=0.6, where approximately 66% of patients had high score and 33% low score. For high score patients and low-score patients, K.M. (Kaplan-Meier) survival curve was plotted for patients from all cancer stages (stage on diagnosis) and also stratified by stage.

Weight loss analysis: For weight loss analysis we excluded patients that undergo either Whipple or Distal Pancreatectomy surgeries and followed patients that had at least 2 weight measurements during the course of disease. N=369. The association between liver enzyme score and the BMI change was evaluated through a linear regression analysis where tumor stage and patient age were controlled. BMI change, ΔBMI, was defined as the (second BMI−first BMI)/first BMI. The linear regression follows the form,

ΔBMI=score+stage+age,

where score stands for liver enzyme score.

Statistical analysis: Unless otherwise specified, all statistical analyses were performed using 2-way ANOVA, Student's t test or Wilcoxon rank sum test of multiple or two groups, with Dunnett's correction when required. The sample size was chosen in advance based on common practice of the described experiment and is mentioned for each experiment. Each experiment was conducted with biological and technical replicates and repeated at least three times unless specified otherwise. The results are presented as mean values±standard deviation. All error bars represent standard deviation. p<0.05 was considered significant in all analyses (* denotes p<0.05, **p<0.005, ***p<0.0005, **** p<0.0001).

Data availability: Due to privacy regulations, all data analysis was conducted on a secured de-identified dedicated server within the Clalit Healthcare environment.

### Example 1: Non-Liver Cancers Induce Early Metabolic Changes in the Liver During Carcinogenesis

To evaluate liver metabolism for changes during carcinogenesis of non-liver cancers, we utilized the orthotopic 4T1-luciferase breast cancer (BC) model that rarely metastasizes to the liver. Using this approach enabled us to differentiate between the liver metabolic changes induced by the primary tumor to those initiated by the metastases. As a control for potential side effects resulting from the orthotopic injection of cancer cells, we injected the mice with sham saline, and in addition, we used the MMTV-PyMT mouse, which is a genetic model of autochthonous BC. We analyzed the UC enzymes and intermediates as a readout of liver-specific metabolic changes in the host during the first 3 weeks of carcinogenesis. We excluded arginase I from our analysis to accurately differentiate the UC from the NO metabolism. In addition, a certified pathologist analyzed and ruled out the existence of metastasis in the livers at all experimental time points.

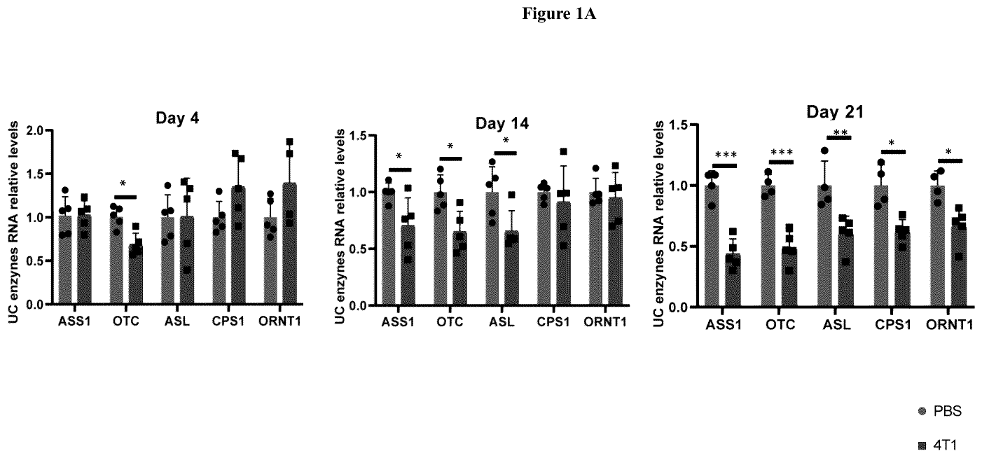

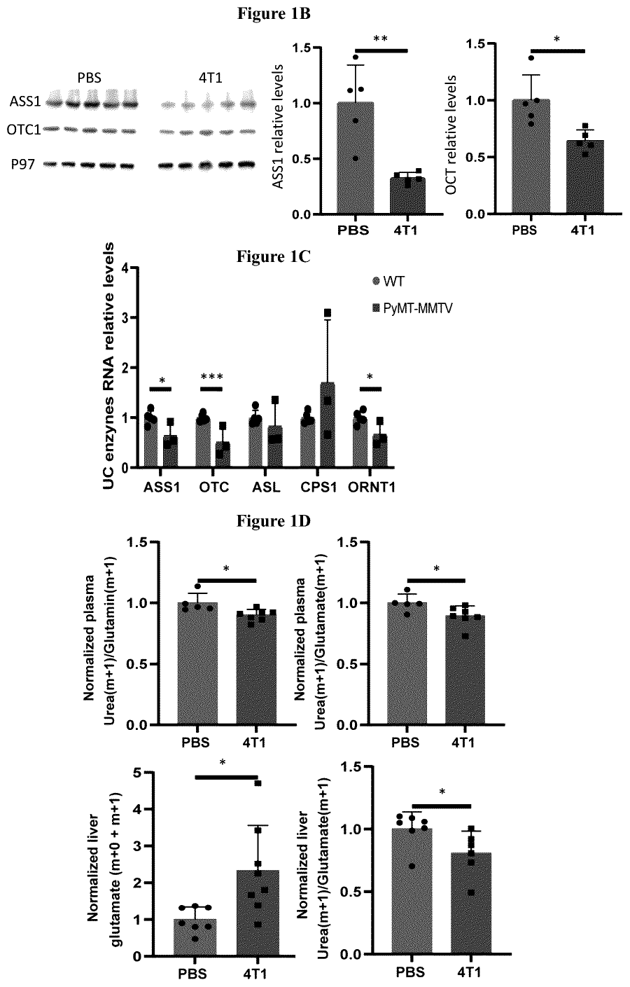

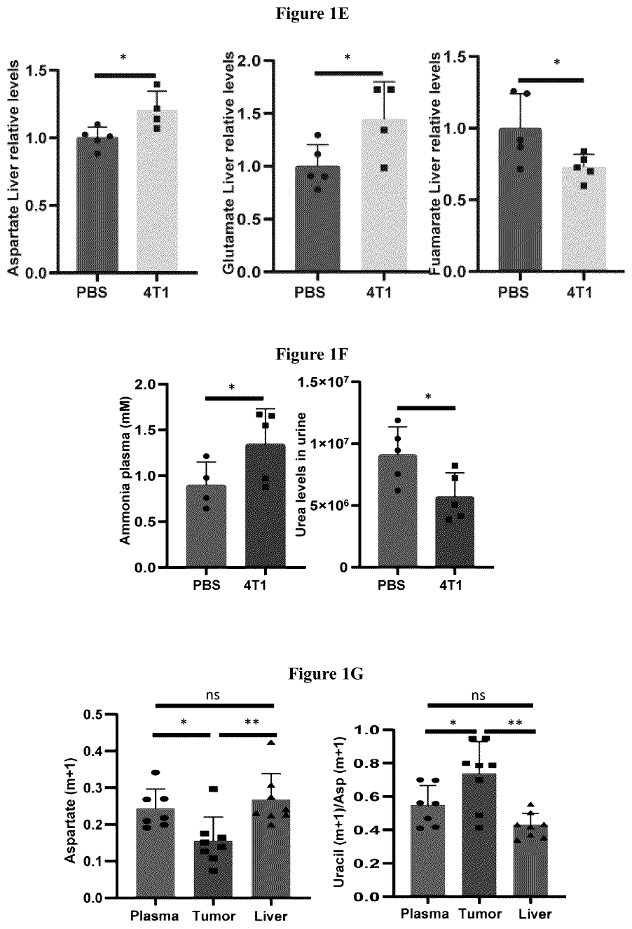

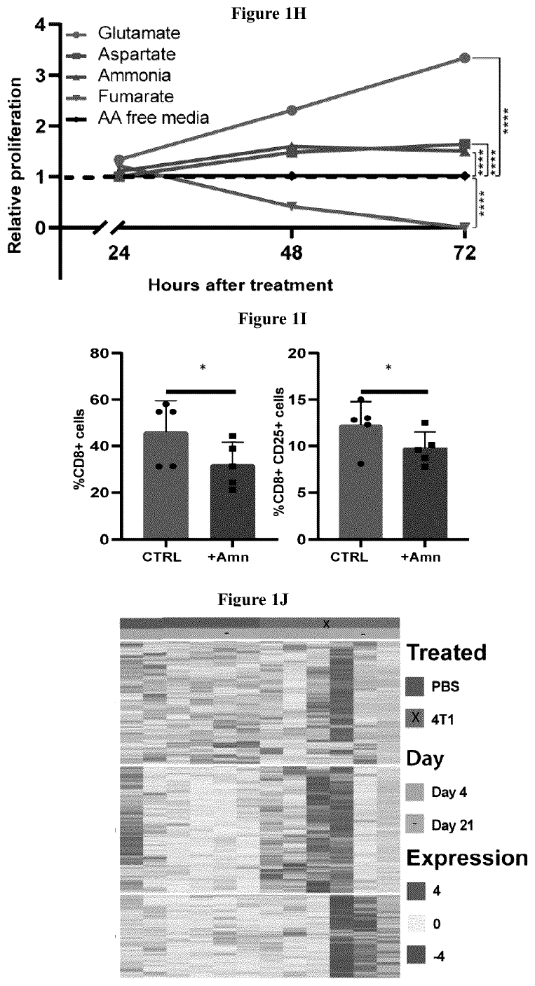

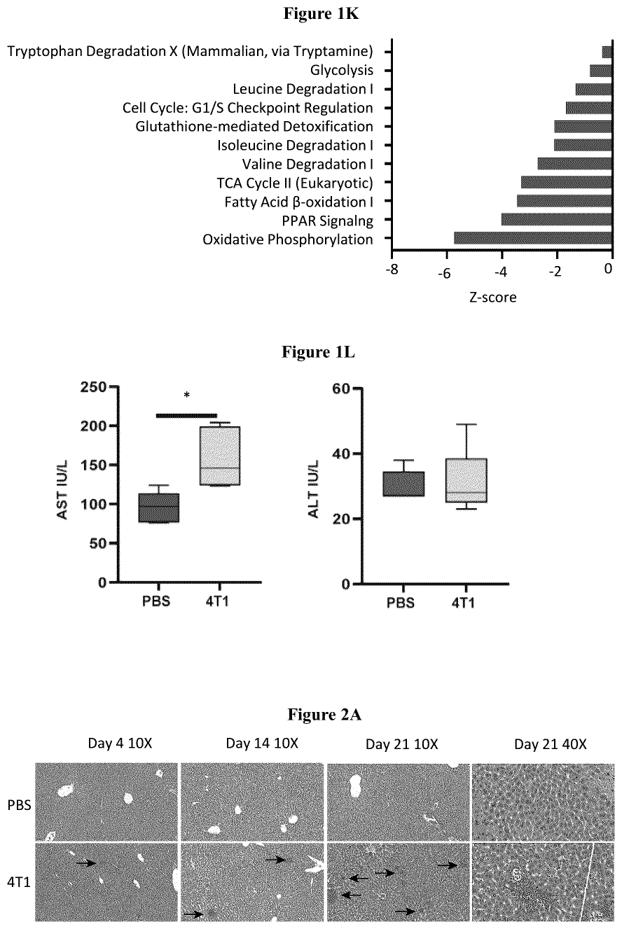

In the 4T1 BC model, we found a decrease in the expression of the UC enzymes in the host's liver, starting as early as day 4 after the orthotopic injection. The decrease in UC enzymes' expression was dynamic along a 3-week course, increasing both in the magnitude of the reduction and the number of enzymes involved (FIG. 1A-B). Interestingly, the genetic MMTV-PyMT BC model also had reduced expression levels of UC enzymes compared to controls, ruling out the possibility that the injection caused the change in UC expression levels (FIG. 1C). To verify the significance of the results in UC expression on UC function, we performed live infusion with ¹⁵N2-labeled glutamine and measured the labeled urea to glutamine and glutamate ratio. We found both these ratios to be decreased in the plasma of breast cancer-bearing mice, and furthermore, in the livers, while total glutamate was elevated, the labeled urea to glutamate ratio was reduced in the 4T1 mice, confirming a malfunctioning UC (FIG. 1D). In addition, we measured UC-related upstream substrates-glutamate and aspartate, and downstream metabolite-fumarate in the plasma and livers of 4T1. As expected from a dysfunctional UC, we found elevated levels of UC substrates, while the levels of fumarate, a UC product, were decreased (FIG. 1E). We further found elevated levels of plasma ammonia and reduced urea levels in the urine, confirming a failing UC (FIG. 1F). Notably, while the levels of the ¹⁵N2-labeled aspartate were lower in the tumor compared to its levels in the plasma and the liver, the ratio of labeled uracil, the downstream metabolite that is synthesized from aspartate, was higher in the tumor, suggesting utilization of aspartate for nucleotide synthesis (FIG. 1G). Indeed, the high levels of UC substrates and the decreased levels of UC products resulting from the depletion of UC function in the host liver directly potentiated cancer-cells proliferation (FIG. 1H). Interestingly, ex-vivo supplementation of ammonia reduced the survival and activation of T-cells splenocytes excised from wild-type (WT) mice (FIG. 1I). Thus, the rewiring of the liver UC during carcinogenesis potentially benefits cancer growth by increasing substrates' availability for tumor growth and by reducing the systemic immune response.

To broadly evaluate whether liver-specific metabolic pathways other than the UC are affected by cancer in-vivo, we performed RNA sequencing of hepatocytes isolated from perfused livers of WT and 4T1 cancer-bearing mice on days 4 and 21 following cancer cells injection. In corroboration of our previous findings, we found a reduction in the expression of UC enzymes Argininosuccinate synthetase (ASS1), Ornithine transcarbamylase (OTC), and Carbamoyl phosphate synthetase I (CPS1), and a significant and distinctive gene expression signature on disease at day 21 (FIG. 1J). In this signature, we found reduced expression of multiple other enzymes participating in central metabolic pathways, supporting a general reprogramming of hepatocyte metabolism during carcinogenesis (FIG. 1K). Further analysis of the hepatocytes RNAseq demonstrated that not all liver genes changed in expression, supporting the specificity of the changes in the metabolic genes. Interestingly, the broad changes in liver metabolism were associated with a significant elevation in AST levels (FIG. 1L).

Since several of the metabolic pathways we found altered, including the UC, depend on adequately functioning mitochondria, we evaluated whether there is a decrease in mitochondrial functions or number following carcinogenesis. In isolated mitochondria, we found a reduction in the activities of respiratory chain complexes that include mitochondrial DNA (mtDNA) encoded subunits (I, II-III, IV) relative to succinate dehydrogenase (SDH, Complex II), which is entirely nuclear-encoded. This finding was corroborated by the observed reduction in liver mtDNA levels in BC-bearing mice. To further understand the perturbation in mitochondrial metabolism, we analyzed the protein and RNA levels of Mitochondrial Transcription Factor A, (TFAM), which regulates mtDNA levels and transcription, and found it significantly downregulated in BC tumor-bearing mice livers. Yet, the total mitochondrial amount, estimated by citrate synthase activity in liver homogenate, was unaltered. Thus, it is likely that transcriptional changes cause the observed metabolic changes rather than mtDNA depletion.

Collectively, this data suggests that in BC mouse models, there are early transcriptional alterations in the expression of metabolic enzymes that cause global changes in liver metabolism at the pathway and organelle levels.

### Example 2: Innate Immune Cells Infiltrate the Liver of the Host During Early Carcinogenesis

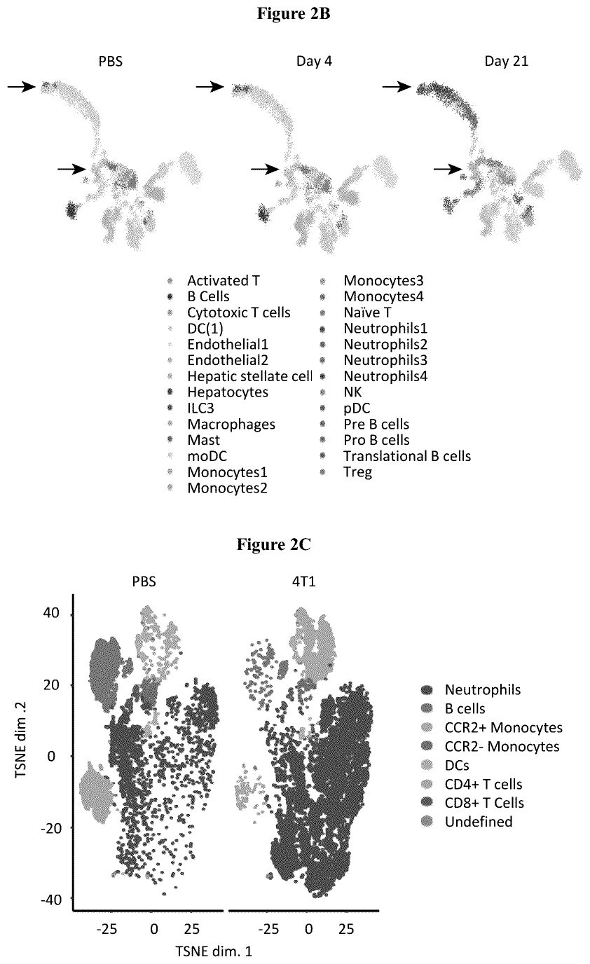

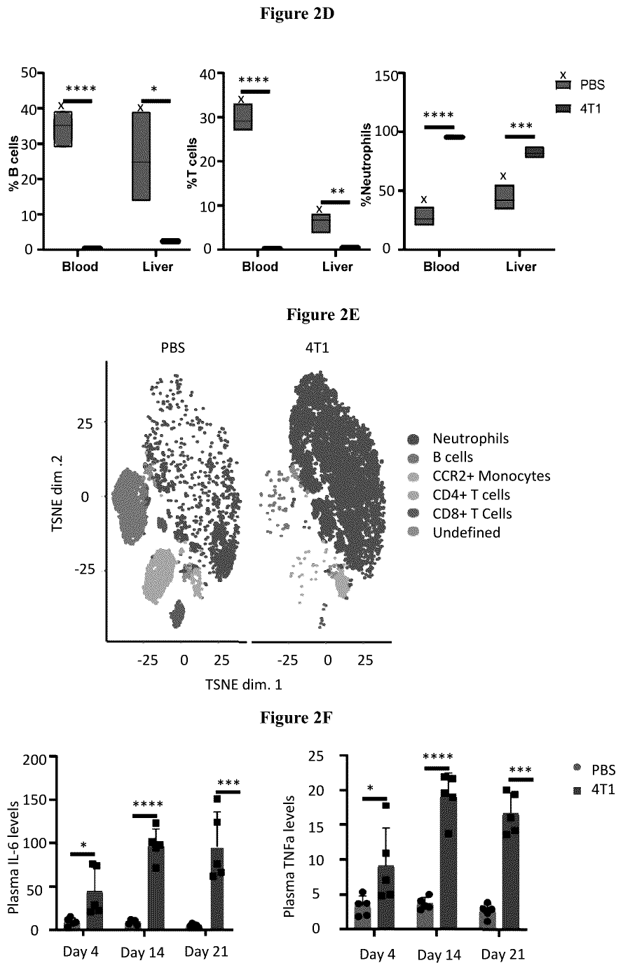

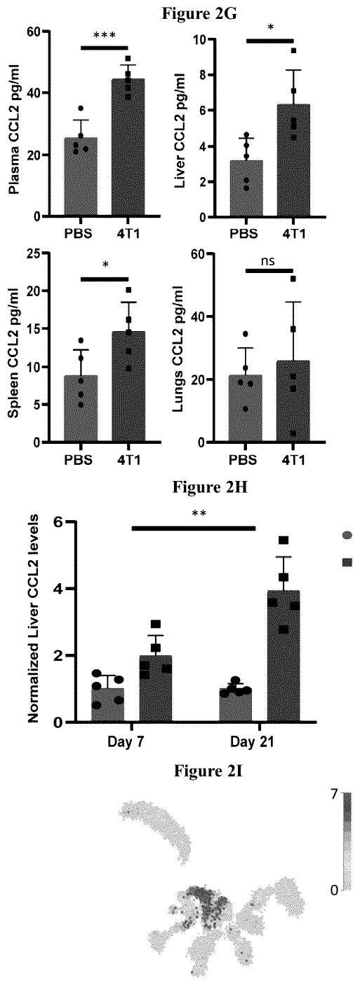

To understand what potentially causes such an early and extensive metabolic rewiring, we first examined the livers for morphological changes following orthotopic injections of BC cells. Interestingly, we found evidence of immune cell infiltration to the livers that start as early as days 4 after BC cell injections and increase along the disease course (FIG. 2A). FACS analysis further confirmed the gradual increase in the liver-immune cells' (CD45+) population in livers of BC-bearing mice between days 0 to 21. Notably, liver histology sections of the MMTV-PyMT genetic BC-model also showed infiltration of immune cells compared to livers of WT mice, supporting that the liver-immune infiltration was not caused by a reaction to the injection.

To identify which immune cells infiltrate the liver, we performed single-cell RNA sequencing (scRNA) analysis of the non-hepatic parenchyma cells in the liver. We complemented the results at the protein level by Cytometry Time Of Flight (CyTOF). Using both technologies, we found a significant decrease in lymphocyte infiltration and an increase in liver infiltrating innate immune cells-neutrophils and monocytes (FIG. 2B-D). The reduction in liver lymphocytes may result from blood lymphopenia due to high sensitivity to elevated ammonia levels (FIG. 2E), which was potentially caused by sensitivity to elevated ammonia levels (FIG. 1I). Conversely, the increased infiltration of innate immune cells to the liver has been described during the formation of pre-metastatic niches or could be part of inflammation-derived extramedullary hematopoiesis. We excluded the option of extramedullary hematopoiesis since we did not find erythrocytes, megakaryocytes, and young granulocytes in the livers. In contrast, we found a significant elevation in the number of mature neutrophils (CXCR2+), and a reduction in CXCR2− neutrophils. Furthermore, FACS analyses excluded bone marrow failure and demonstrated an extensive maturation of neutrophils, correlating with a significant elevation in plasma levels of granulocyte colony-stimulating factor (G-CSF).

It may be that the liver infiltration of immune cells during carcinogenesis increases liver susceptibility to late metastasis formation. Still, our data suggest that liver involvement at this early stage is more likely to be part of a systemic inflammatory response.

Our scRNA data demonstrate that the neutrophils in the liver can be clustered into four subgroups based on substantial differences in gene expression along the time course of carcinogenesis. Neutrophils subset 1, found in the liver at day 4, likely represent immature neutrophils based on the high expression levels of maturation and chemotaxis genes, granules genes, and elevated inflammatory markers IL1P. Following three weeks of carcinogenesis, we found that more mature neutrophils accumulate in the liver in high numbers (subsets 2-4). Additionally, measurement of cytokine levels in the plasma of BC-bearing mice demonstrated a significant increase in IL-6 and TNF-α, supporting the notion that tumorigenesis induces a systemic inflammatory response that involves the liver (FIG. 2F). Because it is usually challenging to detect direct elevation in RNA levels of secreted cytokines in scRNA of in-vivo samples, we analyzed the data for elevations in the RNA of their downstream target. Encouragingly, we found in macrophages at day 21 a significant elevation in the expression of ANXA1, which is known to be driven by IL-6.

The chemokine CCL2 and its primary receptor CCR2 have been linked to the pathogenesis of inflammation and cancer. Indeed, we found elevated levels of CCL2 in livers, plasma, and spleens of BC-mice in the first week following the injection of cancer cells and less so in the lungs (FIG. 2G). Notably, while the increase in plasma CCL2 levels in BC-bearing mice compared to healthy mice remained constant, the relative elevation of CCL2 in the livers of BC-bearing mice continued to increase along the cancer course (FIG. 2H). In addition, we found increased CCR2+ cells in liver infiltrating monocytes and upregulation in the expression of genes involved in migration pathways (FIG. 2I).

Thus, following carcinogenesis, there is an early induction of systemic immune response, in which immune tissues such as the liver secrete increasing levels of CCL2, resulting in infiltration of immune cells to different organs.

### Example 3: pERK Activation in Myeloid Cells Results in the Depletion of HNF4α from Hepatocytes

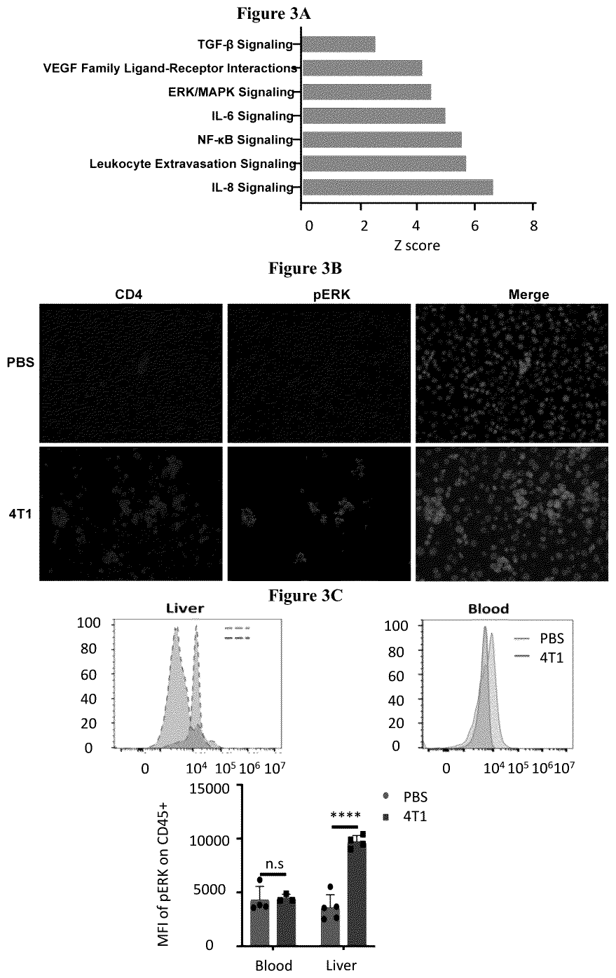

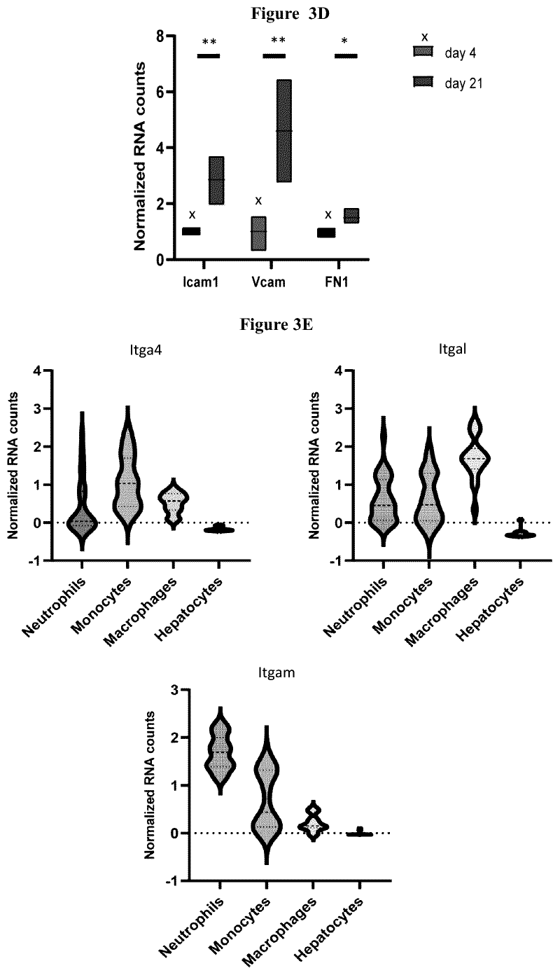

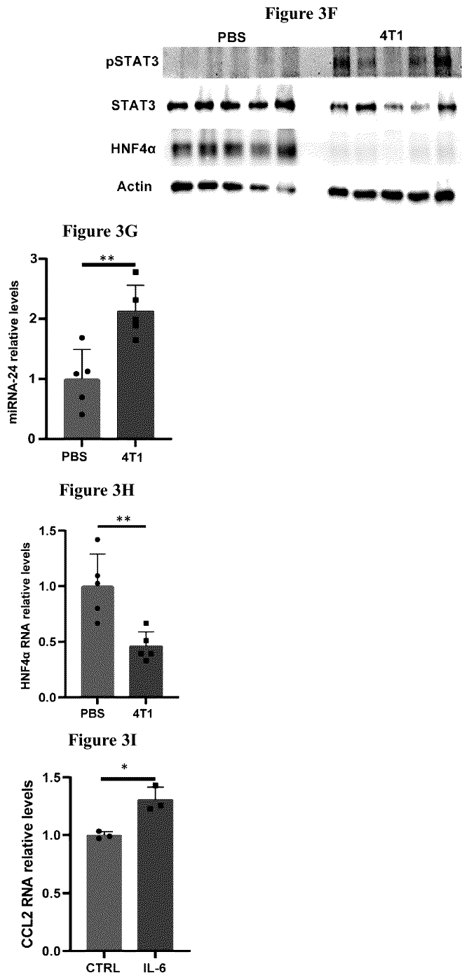

In addition to the alterations we found in metabolic pathways, the bulk RNA sequencing analysis we performed on isolated hepatocytes of BC mice demonstrated an upregulation of signaling pathways (FIG. 3A). IL-6, NFκβ, and ERK, which we found to be elevated in our scRNA pathway analysis, were described previously to participate together as a signaling pathway. In addition, IL-6 was shown to downregulate TFAM, and we found its levels elevated in the plasma of 4T1 mice (FIG. 2F). Thus, we further evaluated the activation state of the ERK-IL-6 pathway in the livers. Immunostaining and FACS analysis of phospho-ERK (pERK) and CD45 cells in the livers of BC-bearing mice revealed increased co-staining of pERK and CD45+ in infiltrating liver cells (FIG. 3B-C). Notably, we did not find pERK activation in the blood cells of BC mice, suggesting that the activation occurred in the liver (FIG. 3C).

Integrins can induce pERK activation upon interaction with other cells. Thus, we performed a ligand-receptor interaction analysis of our scRNA data of liver infiltrating immune cells, together with the bulk RNA sequencing of hepatocytes. We found support for intercellular crosstalk between immune cells and hepatocytes via integrins and their receptors that may stabilize their hepatic localization (FIG. 3D-E). Activated ERK has been shown to cause secretion of IL-6 from myeloid cells with multiple effects on other cells, one of which is a consequent elevation of pSTAT3 levels. Elevated pSTAT3 via mir-24 causes the downregulation of HNF4α, a master regulator of liver metabolism. Indeed, we found significant elevations in pSTAT3 and miR-24 levels in the livers of BC tumor-bearing mice, together with complete depletion of HNF4α, at both the RNA and protein levels (FIG. 3F-H). Notably, supplementing primary hepatocytes with IL-6 increased CCL2 RNA levels (FIG. 3I). Hence, cancer-induced inflammation recruits innate immune cells to the liver at least partly via CCL2. The consequent immune activation and secretion of IL-6 activate pSTAT3 in hepatocytes, leading to HNF4α depletion and perturbation of liver metabolism.

### Example 4: HNF4α Depletion Disrupts Liver Metabolism During Extrahepatic Carcinogenesis

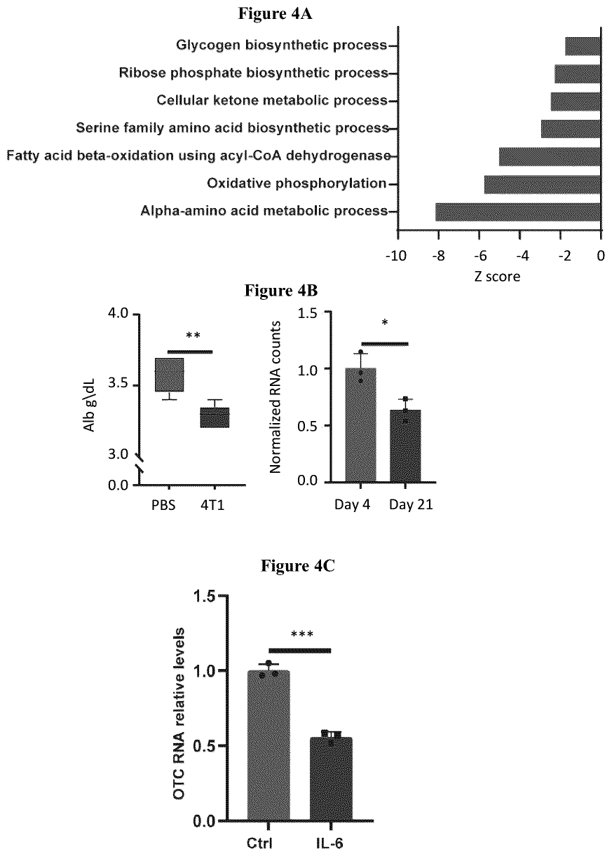

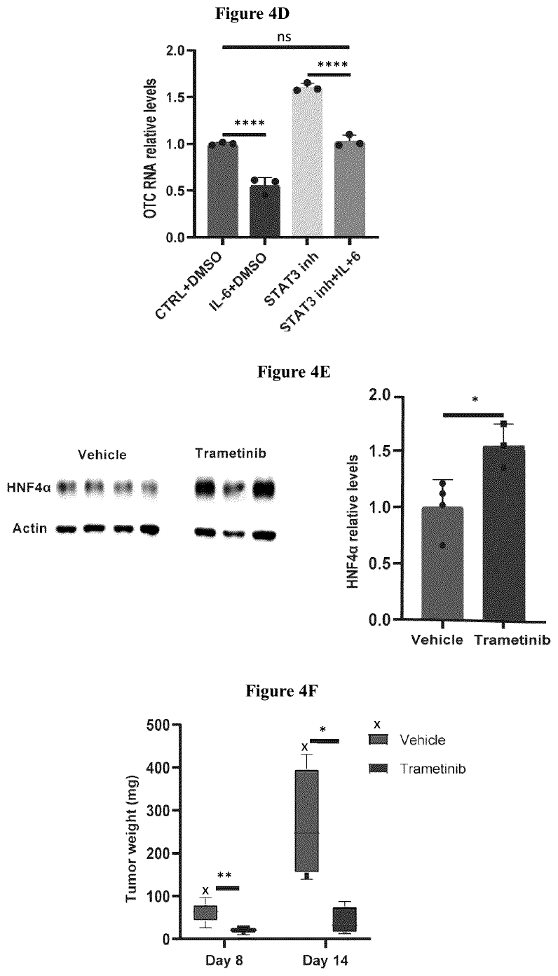

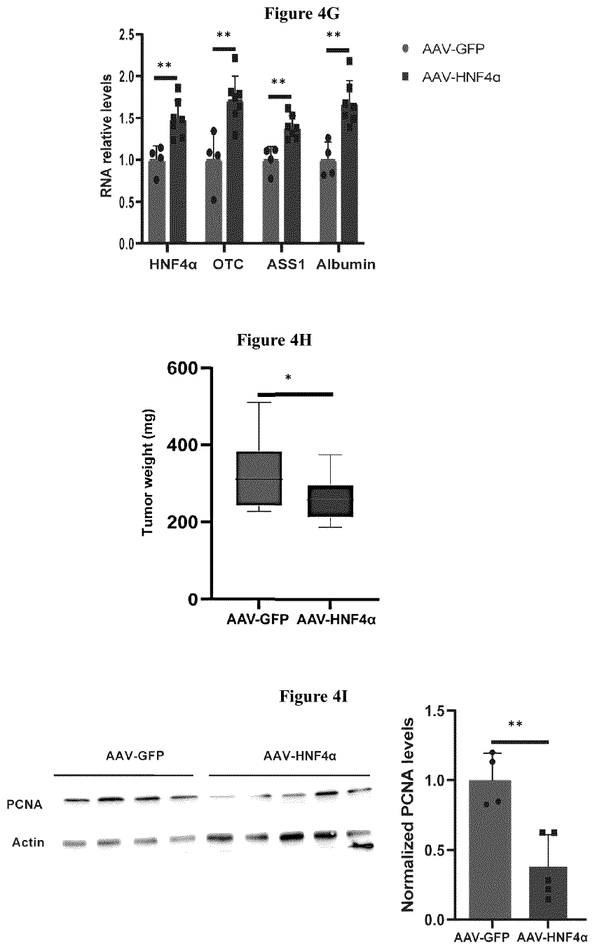

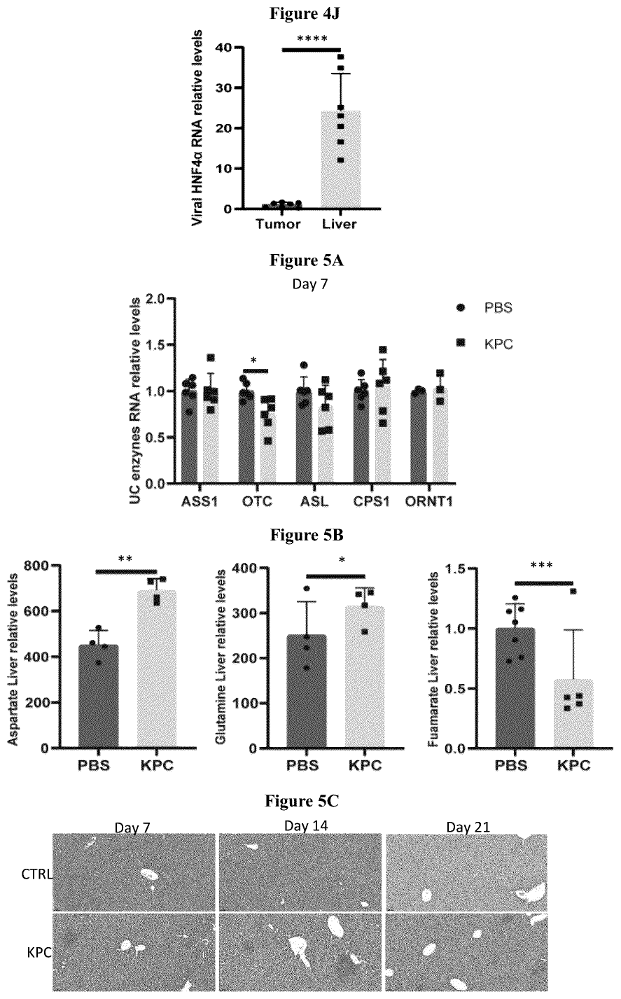

Further analysis of the RNA sequencing data from the livers of BC-mice confirmed that many of the genes we found dysregulated and responsible for the perturbed metabolic and signaling pathways are indeed regulated by HNF4α (FIG. 4A). Since albumin is an established downstream target of HNF4α we confirmed its decrease at both the RNA and protein levels in the liver and plasma of 4T1 mice, respectively (FIG. 4B).

To validate causality between the signaling cascade initiated by immune cells and the consequent metabolic changes in the livers of cancer-bearing mice, we measured the expression levels of the UC enzyme OTC, a known target gene of HNF4α, in isolated primary hepatocytes. We found that IL-6 supplementation decreased the expression levels of OTC and that this effect can be rescued with a STAT3 inhibitor HJC0152 (FIG. 4C-D). While Trametinib (MEK-ERK inhibitor) may have an oncolytic effect, treating 4T1 mice with Trametinib maintained the expression levels of HNF4α and liver UC enzymes and reduced tumor size, further supporting the proposed signaling cascade (FIG. 4E-F).

Finally, in-vivo re-expression of HNF4α via viral transduction in BC mice increased liver HNF4α levels, reversed the changes in the expression of UC enzymes, and restricted BC tumor growth (FIG. 4G-H). Furthermore, tumors from BC mice treated with HNF4α had lower levels of PCNA and lower pCAD, suggesting that preservation of liver metabolism by re-expressing HNF4α decreases tumor growth by restricting proliferation (FIG. 4I). HNF4α injection decreased glutamate and aspartate levels in the liver, plasma, and tumor, suggesting that preserved liver UC metabolism restricted tumor growth. Notably, HNF4α-AAV injection increased the expression of viral HNF4α in the liver but did not increase the levels of viral-HNF4α in the tumors, supporting the liver specificity of the AAV-HNF4 (FIG. 4J). Thus, liver infiltrating immune cells with an activated pERK secrete IL-6, activating pSTAT3 in hepatocytes, leading to HNF4α depletion and subsequently to metabolic perturbations in BC-bearing mice livers. Importantly, re-expressing HNF4α restricted the changes in liver metabolism.

### Example 5: Similar Changes in Liver Metabolism Occur Via HNF4α in Pancreatic Cancer (PC) Mice

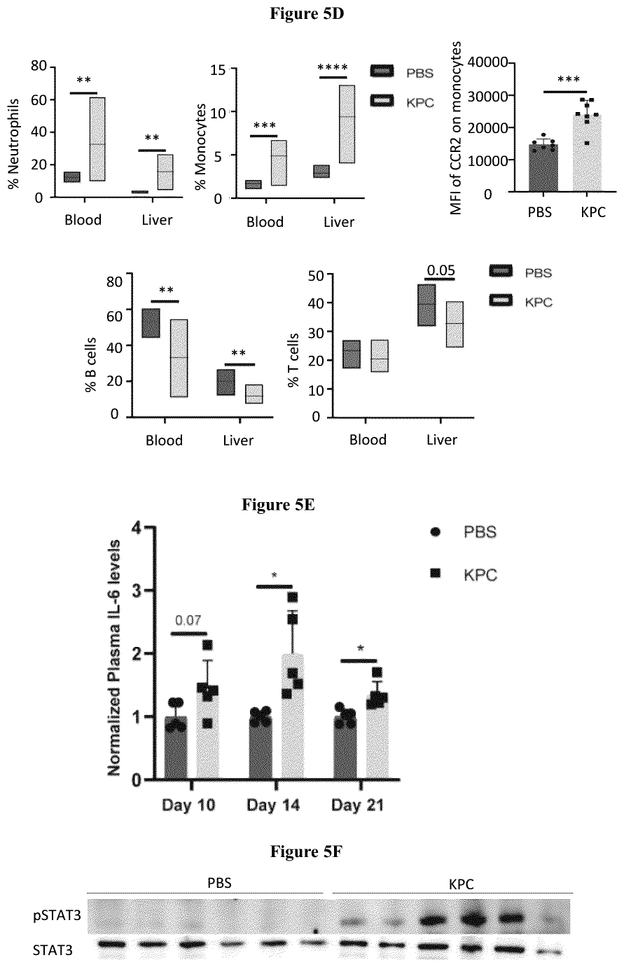

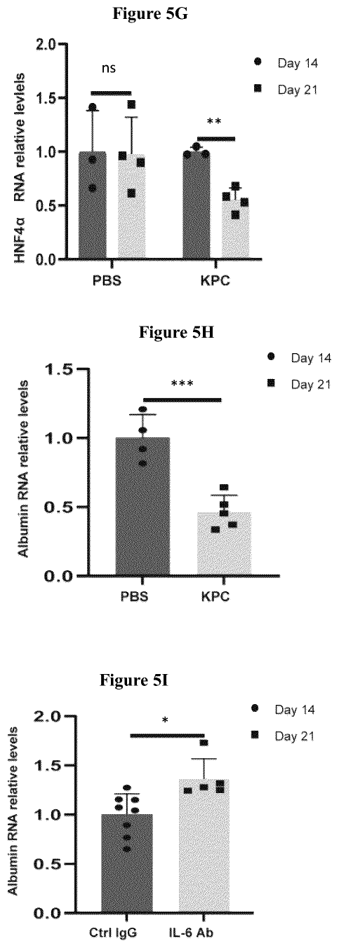

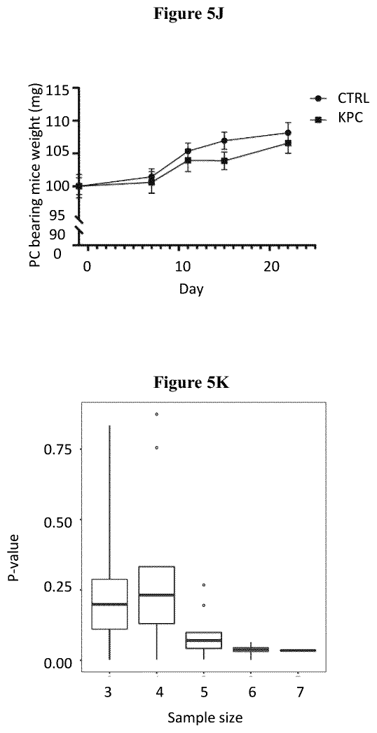

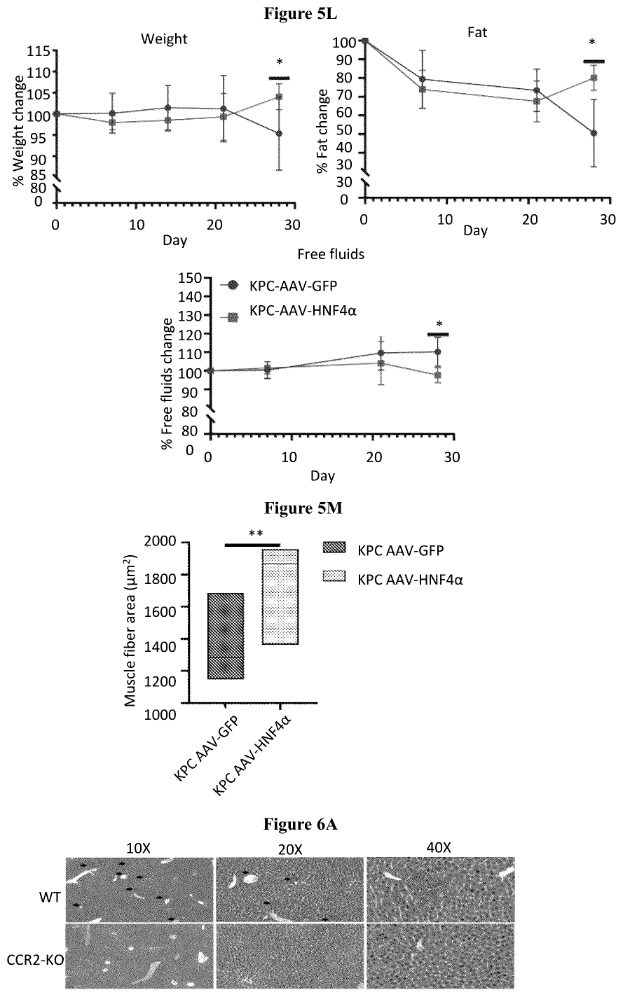

To evaluate our results in another cancer mouse model, we used the KrasG12D/Trp53R172H/Pdx-1-Cre pancreatic cancer (KPC) orthotopic mouse. This mouse is also a known model for cancer induced cachexia. Encouragingly, we found that the early metabolic findings demonstrated in the 4T1 BC model occur in the pancreatic cancer (PC) model. Indeed, we found in the PC model a significant decrease in the levels of OTC, a direct target of HNF4α, already in the first week after injection of KPC cells, as well as an increase in UC substrates and reduced levels of UC products on day 21 (FIG. 5A-B). Additionally, we found in the PC mice a gradual increase in immune cell infiltration to the liver during the first 3 weeks of tumorigenesis (FIG. 5C). FACS analysis confirmed that the liver infiltrating immune cells were predominantly of innate immunity and associated with increased CCR2+ monocytes and lymphocyte depletion (FIG. 5D). Consistent with our findings in the BC model, our results in the KPC model were associated with an increase in the plasma levels of IL-6, elevation in pSTAT3 protein levels, a decrease in HNF4α RNA levels, and consequently decreased albumin levels (FIG. 5E-H). In further support, treating KPC mice with anti-IL-6 antibodies rescued albumin levels (FIG. 5I). Notably, these changes in liver metabolism occurred when there were no changes in the weight of the KPC mice (FIG. 5J) and when a certified pathologist ruled out liver metastasis. Thus, KPC mouse model demonstrates changes in liver metabolism that are similar to the ones we describe in BC mouse models.

### Example 6: CCR2 KO and HNF4α Re-Expression Rescue the Early Metabolic Changes in the Liver During Carcinogenesis in PC Mice

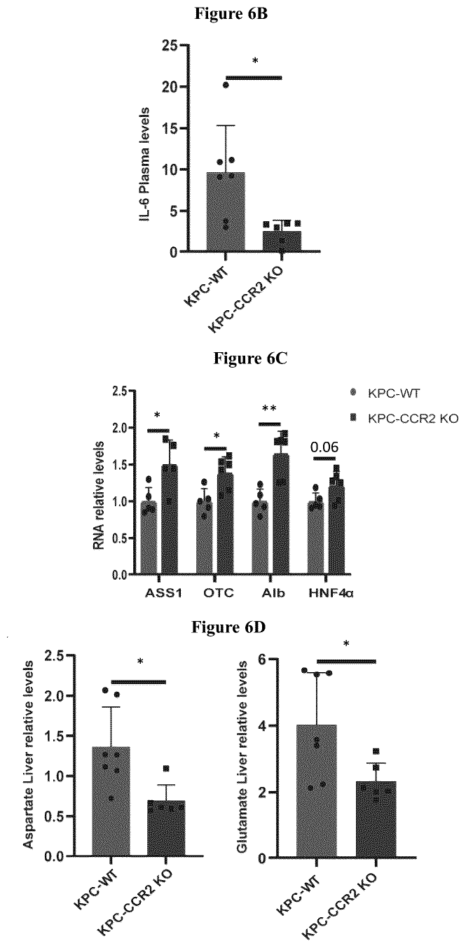

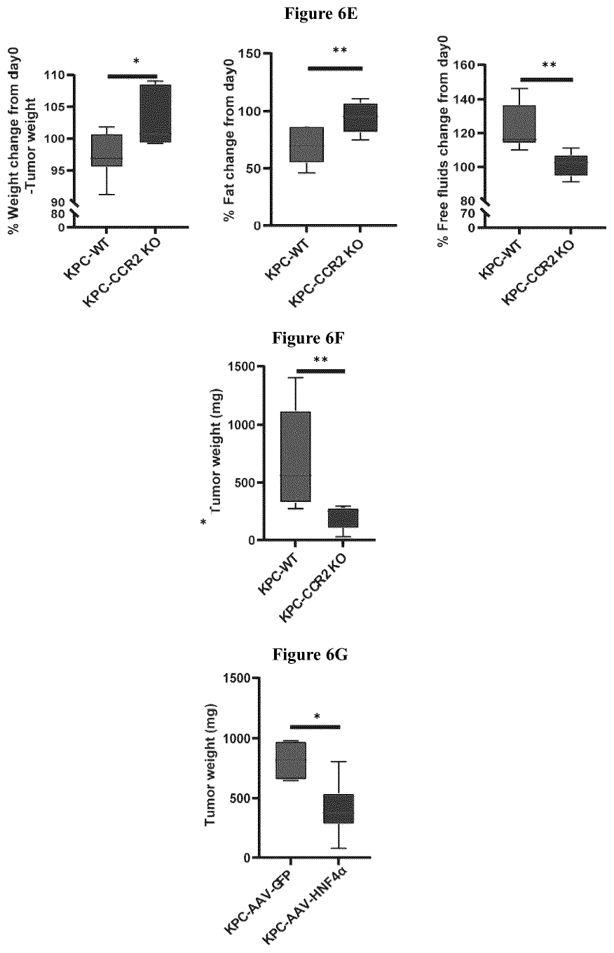

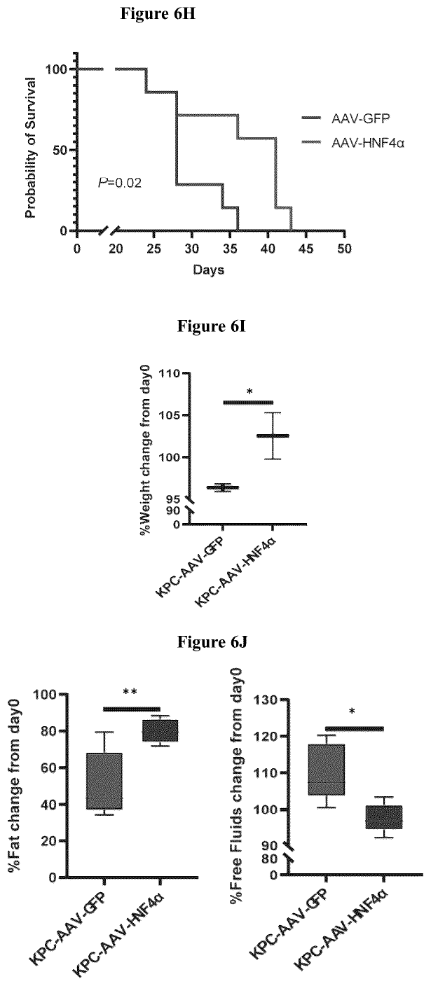

To further confirm that CCL2 drives the immune infiltration to the liver and is responsible for the metabolic changes we find, we orthotopically injected the KPC cells into C57/B16 wild-type and CCR2^{-/-} knockout mice, which do not express the receptor for CCL2 and cannot recruit CCL2+ immune cells. In contrast to KPC CCR2^{+/+} mice, we found that the KPC CCR2^{-/-} mice developed PC but did not demonstrate liver infiltration by immune cells (FIG. 6A). Consequently, we found decreased levels of IL-6 in the plasma of PC CCR2^{-/-} mice (FIG. 6B), while the expression levels of UC enzymes, albumin, and HNF4α were preserved (FIG. 6C). Notably, the CCR2^{-/-} mice had reduced levels of UC substrates in their livers and did not develop systemic metabolic manifestations associating with carcinogenesis such as weight loss and changes in body composition, correlating with the preservation of albumin level. Importantly, CCR2−/− mice had significantly decreased tumor growth (FIG. 6D-F).

Finally, to evaluate the potential therapeutic relevance of our findings, we injected the AAV8-HNF4α virus into KPC mice. AAV8-HNF4α virus significantly restricted PC tumor growth (FIG. 6G) and improved survival (FIG. 6H).

Notably, re-expressing HNF4α also reduced CAC phenotypes such as weight loss (FIG. 6I), decreased fat loss (FIG. 6J), and restricted the accumulation of body fluids (FIG. 6J), as demonstrated with body composition analysis by NMR (5K-M).

Thus, preserving the levels of HNF4α in the liver by preventing CCL2+ immune cell infiltration or by re-expressing HNF4α alleviated the systemic manifestations of tumorigenesis, such as weight loss and changes in body composition.

### Example 7: A Biochemical Liver Score Based on Routine Tests can Predict Outcomes in BC and PDAC Patients, Including Cachexia

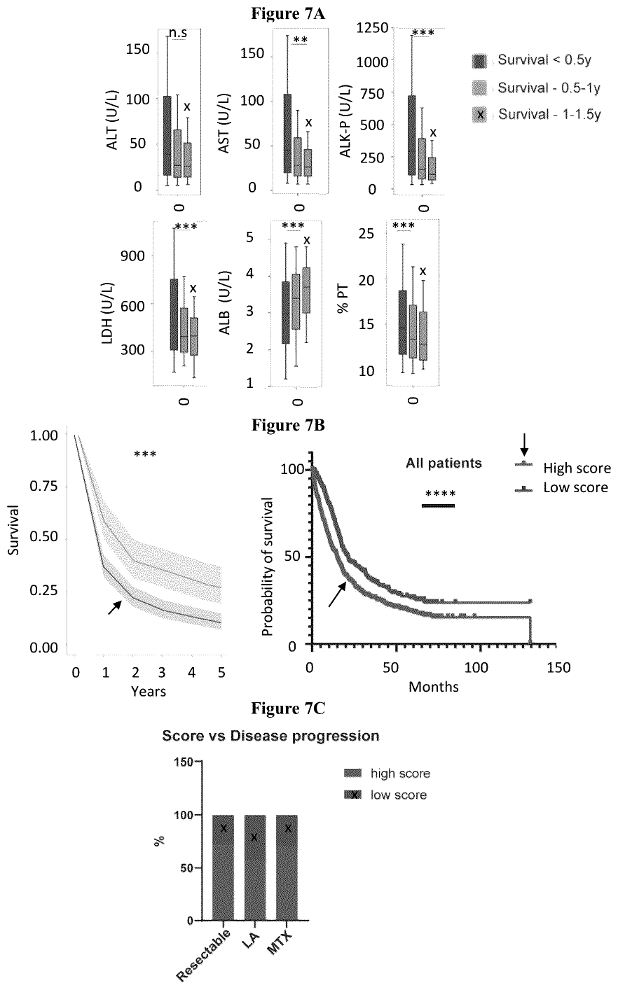

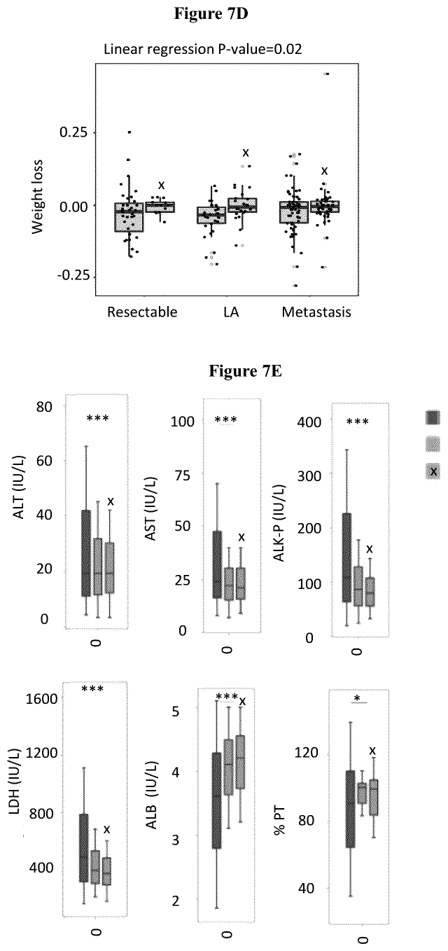

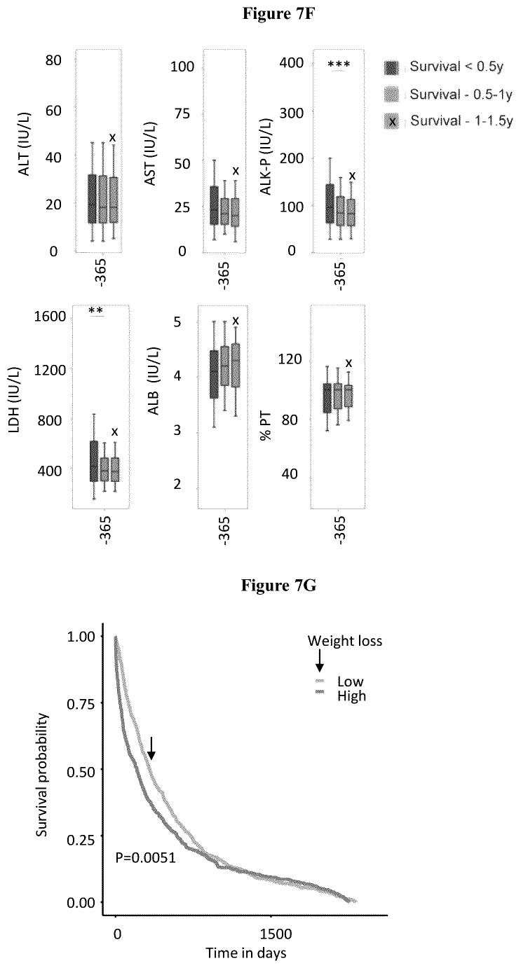

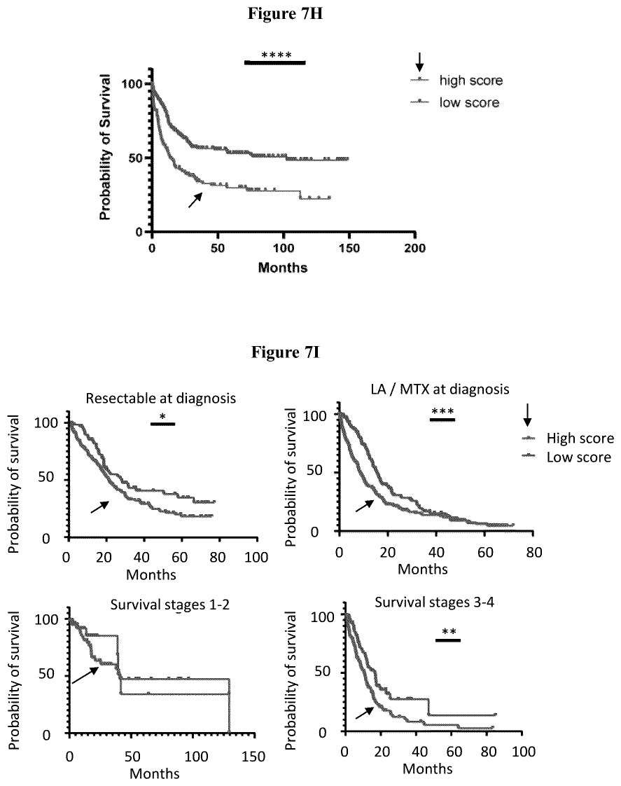

To understand the translational relevance of our findings in liver metabolism for cancer patients, we performed analyses of the Clalit health maintenance organization (HMO) dataset, which encompasses digital health data of 5 million Israeli subjects for 18 years. We found that patients with non-metastatic BC and pancreatic ductal adenocarcinoma (PDAC) with abnormal liver parameters on the day of diagnosis survive for a shorter time than patients with normal liver parameters (FIG. 7A, 7E). Notably, some of these liver changes such as LDH and ALP, could be significantly detected in the plasma of BC patients and predict the outcomes, even a year before diagnosis (FIG. 7F).

In addition, we analyzed datasets of PDAC patients specifically, from two independent medical centers in Israel—the Sheba and Souraski Medical Centers, two of Israel's largest oncology centers. We first confirmed that this cohort behaves as published in the literature and shows a correlation between decreased survival and weight loss (FIG. 7G). Similar to a score we previously generated for the expression of UC enzymes (see Lee et al., “Urea cycle dysregulation generates clinically relevant genomic and biochemical signatures”, Cell. 2018 Sep. 6; 174(6): 1559-1570.e22, herein incorporated by reference in its entirety), we now developed a “liver-score” that is based on measured levels of liver biochemical and functional parameters (AST, ALT, ALP, albumin, INR), and trained it on a small dataset (FIG. 7H). We next tested the correlation potential of our score with survival in the Clalit and the hospitals' data sets. We found that in all three cohorts, our “liver-score” correlates with decreased survival in PDAC patients (FIG. 7B). Interestingly, the liver score did not correlate with the disease stage but did correlate with weight loss (FIG. 7C-D, 7I).

The depletion of HNF4α can initiate the cascade of events that lead to muscle protein breakdown in CAC via decreasing albumin levels. Analysis of the data from Sheba Medical Center and Souraski Medical Center showed that the change in PC patients' BMI, significantly associated with the liver enzyme score, once the tumor stage and patient age are controlled for. Thus, the decreased survival predicted by our liver-score coincides with systemic manifestations induced by cancer development including cachexia.

In summary, we demonstrate that molecular and functional systemic metabolic changes occur in the liver during early extrahepatic carcinogenesis, even before the clinical manifestations. These metabolic changes are mediated by innate immune cells, resulting in activation of pERK signaling, leading to the depletion of HNF4α the master metabolic regulator in hepatocytes. The perturbation of multiple liver metabolic pathways contributes to carcinogenesis, immune evasion, and eventually to the development of CAC (FIG. 8).

Further support is also provided in Goldman et al., “Early infiltration of innate immune cells to the liver depletes HNF4α and promotes extra-hepatic carcinogenesis”, 2023, Cancer Discov. 2023 Mar. 27; CD-22-1062, herein incorporated by reference in its entirety.

### Example 8: LNP Delivery of HNF4α Specifically to Liver Cells Both Treats and Prevents CAC

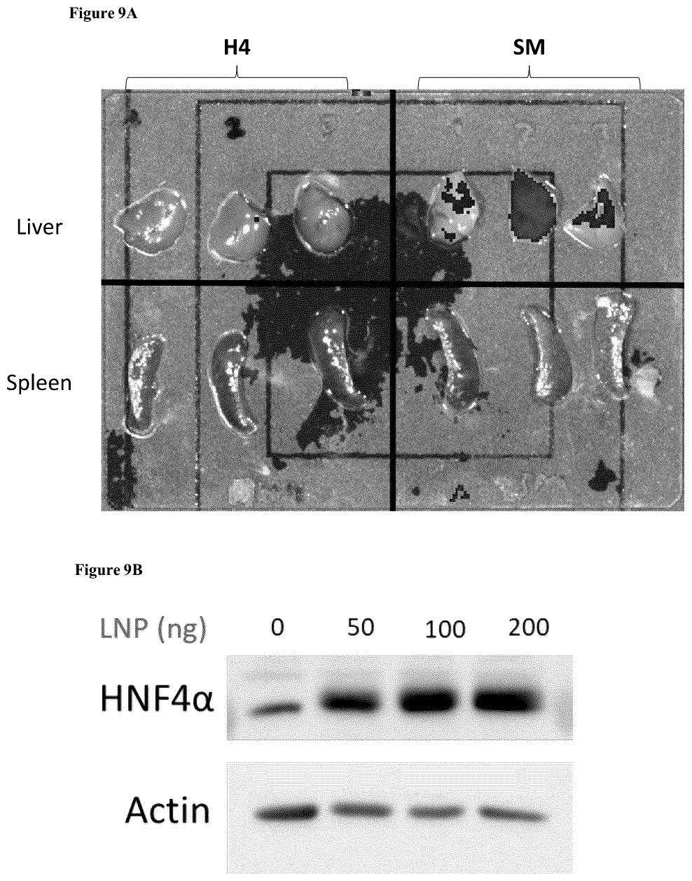

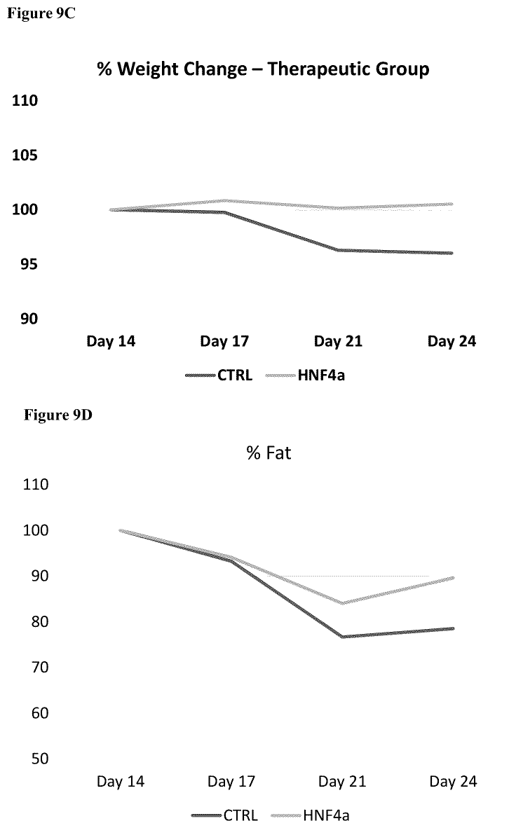

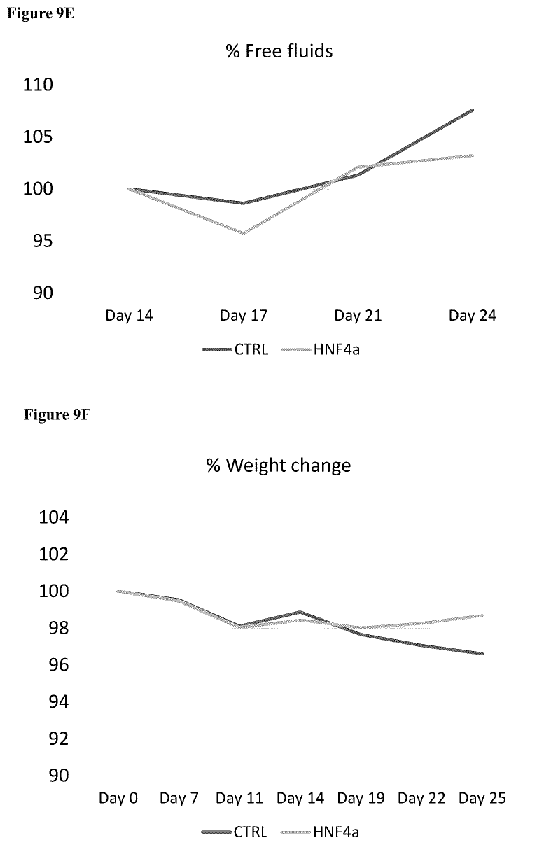

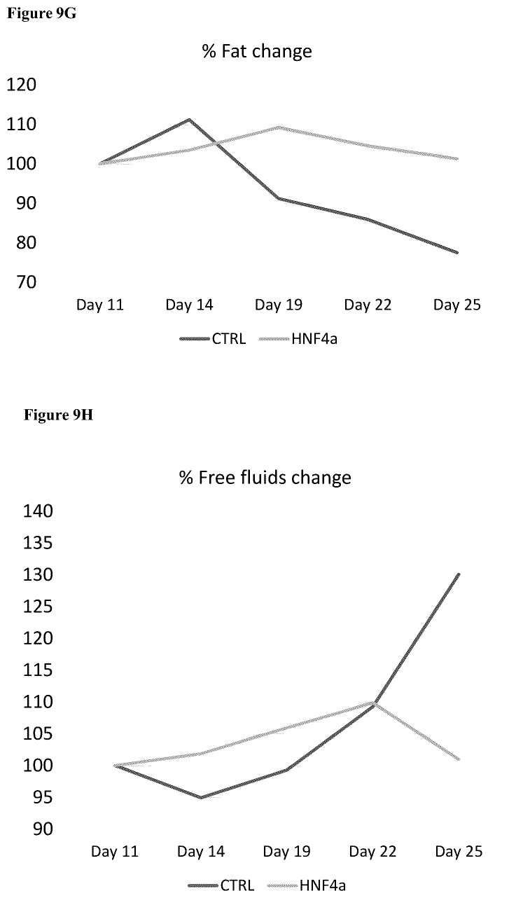

Though AAV delivery of HNF4α was able to prevent development of the symptoms of CAC, direct AAV delivery to humans is not feasible. As such, lipid nanoparticles (LNPs) were designed for the delivery of an HNF4α mRNA to the liver. The first LNP, herein called SM-LNP, had the following composition: 50 mol % SM-102, 38.5 mol % cholesterol, 10 mol % DOPE, and 1.5 mol % DMG-PEG200. The second LNP, herein called H4-LNP, had the same composition as the first LNP but used a different ionizable lipid (not SM-102). The lipids were dissolved in ethanol and the LNP was generated by ethanol injection. The ethanol was mixed (at a ratio of 1:3) with an aqueous solution (pH 5.2) containing the mRNA at a nitrogen (in the lipid headgroup) to phosphate (in the RNA) ratio (N:P) or about 8. LNPs generated with mRNAs coding from emGFP were used to test the biodistribution of the LNPs. Though both LNPs were predicted to target to the liver, only the SM-LNP produced fluorescence in the liver, while the H4-LNP did not (FIG. 9A). Neither LNP composition produced detectable expression in the spleen. The SM-LNP was thus selected for further experimentation due to its high selectivity for the liver.

A HNF4A encoding mRNA was designed for inclusion within the LNP. The mouse spliced mRNA coding sequence was used (SEQ ID NO: 2), although the human sequence (SEQ ID NO: 5) can be used as well. Capping and ribosome binding domains were included in the 5′ UTR (SEQ ID NO: 7) and a poly-adenylated 3′ UTR (SEQ ID NO: 8) was included as well. The 5′ UTR was designed for high expression and included a T7 RNA promoter sequence (SEQ ID NO: 46) at the very 5′ end, and a sequence from the 5′ UTR of human alpha globin mRNA (HBA1) which included a Kozak consensus sequence. A 3′ UTR from mitochondrial rRNA sequence (SEQ ID NO: 8) was selected as it had high thermodynamically stable secondary structure under LNP formation temperature conditions (ΔG=−85.3 kcal/mole, calculated using UNAFold for two state folding). Following loading of the mRNA into the SM-LNP its ability to induce expression of HNF4A protein in hepatocytes was tested. Cells of THLE-2 human hepatocyte cell line were incubated with increasing concentrations of the LNP and after 16 hours cells were lysed and wester blotting for HNF4A was performed. Robust, dose-dependent, expression of HNF4A was observed in the cells (FIG. 9B) indicating that the mRNA was functional in hepatocytes.

The therapeutic and preventative potential of the LNPs with respect to cachexia were tested in a PC mouse model. KPC cells were injected orthotopically into the pancreas of mice at day 0 and a tumor was allowed to develop. With no intervention, symptoms of CAC including weight loss, fat loss and increased free fluids were clearly observable in the mice by day 21 (FIG. 9C-E, Control group). In the treatment group, 6.5 mcg of LNP was administered intravenously into the tail vein at day 21 and already by day 24 a halt/slowing to the cachexic changes was observed (FIGS. 9C-E, HNF4A group). As described hereinabove, metabolic changes can be detected early on in cancer progression, before cachexia has developed. This allows for preventative restoration of HNF4A expression in the liver, which would preempt the development of cachexia. In the prevention group, the LNP was administered at day 7, before symptoms of cachexia are observable (but after metabolic changes have begun, see FIGS. 1A and 5A). This prophylactic treatment completely abolished the development of cachexic symptoms, as there was no significant decrease in weight, fat or free fluids as of day 25 after the cancer was administered (FIGS. 9F-H). These results demonstrate the therapeutic and prophylactic efficacy of HNF4A delivery to the liver.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1.-31. (canceled)

32. A method of detecting a non-hepatic cancer in a subject or predicting a clinical outcome in a subject suffering from non-hepatic cancer, the method comprising measuring function of the urea cycle in said subject, wherein decreased urea cycle function as compared to urea cycle function in a healthy control indicates said subject suffers from a non-hepatic cancer or indicates a worse clinical outcome for a subject suffering with non-hepatic cancer as compared to a subject without decreased urea cycle function, thereby detecting non-hepatic cancer or predicting a clinical outcome in a subject suffering from non-hepatic cancer, further comprising administering to a subject identified with said worse clinical outcome and determined to have non-hepatic cancer, at least one therapeutic agent selected from: an anti-IL6 blocking antibody, an ERK inhibitor, a STAT3 inhibitor and an agent capable of increasing expression of HNF4A in a liver of said subject.

33. The method of claim 32, wherein said non-hepatic cancer is selected from breast cancer and pancreatic cancer, does not comprise detectable metastasis to the liver or both.

34. The method of claim 32, wherein measuring function of the urea cycle comprises at least one of:
a. measuring in the liver of said subject expression of at least one urea cycle enzyme selected from: argininosuccinate synthetase 1 (ASS1), ornithine transcarbamoylase (OTC), argininosuccinate lyase (ASL), carbamoyl phosphate synthetase-1 (CPS1) and ornithine translocase (ORNT1/SLC25A15);
b. measuring urea to glutamine ratio or urea to glutamate ratio in the blood or liver of said subject;
c. measuring glutamate, aspartate or fumarate levels in the blood or liver of said subject;
d. measuring ammonia levels in the blood of said subject;
e. measuring urea levels in the urine of said subject;
f. measuring hepatocyte nuclear factor 4 alpha (HNF4A) expression in the liver of said subject;
g. measuring in the blood of said subject levels of at least one liver enzyme selected from: aspartate aminotransferase (AST), alanine aminotransferase (ALT), alkaline phosphatase (ALK-P/ALP), and lactate dehydrogenase (LDH);
h. measuring the prothrombin time international normalized ratio (INR) of blood from said subject; and
i. measuring albumin in the blood or liver of said subject.

35. The method of claim 34, wherein
a. a decrease in expression of said at least one urea cycle enzyme;
b. a decrease in urea to glutamine ratio or urea to glutamate ratio;
c. an increase in glutamate or aspartate;
d. a decrease in fumarate;
e. an increase in ammonia;
f. a decrease in urea;
g. a decrease in HNF4A;
h. an increase in levels of said at least one liver enzyme;
i. a decrease in INR;
j. a decrease in albumin; or
k. any combination thereof
is indicative of decreased urea cycle function in said subject.

36. The method of claim 32, wherein said measuring function comprises producing a liver-function score and wherein a liver-function score beyond a predetermined threshold indicates decreased urea cycle function.

37. The method of claim 36, wherein said liver-function score is a weighted sum of normalized levels of AST, ALT, ALP, and albumin and INR in a blood sample from said subject, optionally wherein said score is standardized from 0 to 1, said predetermined threshold is 0.6 and wherein a score above said predetermined threshold indicates decreased urea cycle function.

38. The method of claim 32, wherein said clinical outcome is development of cancer-associated cachexia and wherein decreased urea cycle function is predictive of an increased risk of developing said cancer-associated cachexia.

39. The method of claim 36, wherein said clinical outcome is overall survival and wherein liver-function score beyond a predetermined threshold indicates a reduced overall survival time.

40. The method of claim 32, wherein said non-hepatic cancer is selected from breast cancer and pancreatic cancer, does not comprise detectable metastasis to the liver or both.

41. A synthetic lipid nanoparticle (LNP) comprising encapsulated therein an mRNA encoding for HNF4A, wherein:
a. said lipid nanoparticle comprises SM-102 lipid, cholesterol, DOPE, and DMG-PEG;
b. said mRNA comprises or consists of a sequence of SEQ ID NO: 10 or 12 or a sequence comprising at least 85% identity thereto and encoding HNF4A and a poly-A tail; or
c. both.

42. The synthetic LNP of claim 41, wherein said mRNA comprises a 5′ cap and a poly-A tail.

43. The synthetic LNP of claim 41, wherein said mRNA encoding for HNF4A comprises the mRNA coding sequence of SEQ ID NO: 2 or SEQ ID NO: 5 or a sequence with at least 85% identity thereto which encodes for HNF4A.

44. The synthetic LNP of claim 41, wherein said lipid nanoparticle targets to liver cells, comprises about 50 mol % SM-102, 38.5 mol % cholesterol, 10 mol % DOPE, and 1.5 mol % DMG-PEG200 or both.

45. A pharmaceutical composition comprising the synthetic LNP of claim 41 and a pharmaceutically acceptable carrier excipient or adjuvant.

46. A method of treating a non-hepatic cancer in a subject in need thereof, the method comprising administering to said subject an agent capable of increasing expression of HNF4A in a liver of said subject or of preventing cancer-associated cachexia in a subject in need thereof, the method comprising administering to said subject a composition comprising at least one agent selected from: an anti-IL6 blocking antibody, an ERK inhibitor, a STAT3 inhibitor and an agent capable of increasing expression of HNF4A in a liver of said subject, thereby treating said non-hepatic cancer in a subject.

47. The method of claim 46, wherein said agent comprises a nucleic acid molecule encoding said HNF4A.

48. The method of claim 47, wherein at least one of:
a. said nucleic acid molecule is contained within an adeno-associated virus (AAV);
b. said nucleic acid molecule is an mRNA;
c. said nucleic acid molecule is an mRNA comprising a 5′ cap and a poly-A tail;
d. said nucleic acid molecule comprises or consists of SEQ ID NO: 10 or 12 or comprising at least 85% identity thereto and encoding HNF4A; and
e. a combination thereof.

49. The method of claim 46, wherein said agent is a synthetic lipid nanoparticle (LNP) comprising encapsulated therein an mRNA encoding for HNF4A, wherein:
a. said lipid nanoparticle comprises SM-102 lipid, cholesterol, DOPE, and DMG-PEG;
b. said mRNA comprises or consists of a sequence of SEQ ID NO: 10 or 12 or a sequence comprising at least 85% identity thereto and encoding HNF4A and a poly-A tail; or
c. both.

50. The method of claim 46, wherein said subject suffers from early-stage cancer, a pre-cancerous lesion or is at risk of developing cancer.

51. The method of claim 46, wherein said subject is determined to have an increased risk of developing said cancer-associated cachexia by a method of claim 38.
